(19) United States
(12) Patent Application Publication
(10) Pub. No.: US 2007/0048738 A1
(43) Pub. Date: Mar. 1, 2007

(54) Methods and compositions for diagnosis, staging and prognosis of prostate cancer

(75) Inventors: Krishna V. Donkena, Rochester, MN (US); Charles Y.F. Young, Rochester, MN (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Mayo Foundation for Medical Education and Research, Rochester, MN (US)

(21) Appl. No.: 10/564,585

(22) PCT Filed: Jul. 14, 2004
(86) PCT No.: PCT/US04/22850

§ 371 Date: Aug. 18, 2006

Related U.S. Application Data
(60) Provisional application No. 60/487,553, filed on Jul. 14, 2003.

(51) Int. Cl. C12Q 1/68

(52) U.S. Cl. 435/6

(57) ABSTRACT

The present invention provides, inter alia, novel methods and compositions for the diagnosis, staging and prognosis of prostate cancer, based on DNA methylation and/or modulation of gene expression, including transcriptional silencing. Preferred diagnostic and/or prognostic nucleic acid and protein markers include at least one of: the differentially (relative to benign tissue) down-regulated sequences corresponding to zinc finger protein 185 (ZNF 185), prostate secretory protein (PSP94), bullous pem-phigoid antigen (BPAG), supervillin (SVIL), proline rich membrane anchor 1 (PRIMA1), TU3A, FLJ14084, KIAA1210, Sorbin and SH3 domain containing 1 (SORBS1), and C21orf63; and the differentially up-regulated sequences MARCKS-like protein (MLP) SRY (sex determining region Y)-box 4 (SOX4), fatty acid binding protein 5 (FABP5), MAL2, and Erg-2.

### CROSS-REFERENCE TO RELATED APPLICATION

This application claims the benefit of priority to U.S. Provisional Application No. 60/487,553 filed 14 Jul. 2003, and incorporated herein by reference in its entirety.

### STATEMENT REGARDING FEDERALLY FUNDED RESEARCH

This work was, at least in part, supported by National Institutes of Health Grants CA91956 and CA70892, and the United States Government has certain rights in the invention.

### FIELD OF THE INVENTION

The present invention relates to novel methods and compositions for the diagnosis, staging, prognosis and treatment of prostate cancer, based on genomic markers for genomic DNA methylation and/or gene expression, including transcriptional silencing, and/or based on protein markers. Particular embodiments provide methods, nucleic acids, nucleic acid arrays and kits useful for detecting, or for detecting and differentiating between or among prostate cell proliferative disorders and/or tumor progression.

### BACKGROUND

Currently, tumor stage, Gleason score, and preoperative serum PSA are the only well-recognized predictors of prostate cancer progression. However, these markers cannot reliably identify men that ultimately fail therapy, and give no insight into prostate carcinogenesis, or potential therapeutic targets for prostate cancer.

Prostate cancer initiation and progression are processes involving multiple molecular alterations, including alteration of gene, and gene product expression. Identification of these differentially expressed genes represents a critical step towards a thorough understanding of prostate carcinogenesis and an improved management (e.g., diagnostic and/or prognostic) of prostate cancer patients.

Inactivation of tumor suppression genes is an important event contributing to the development of neoplastic malignancies. In addition to the classical genetic mechanisms involving deletion or activating point mutations, growth regulatory genes can be functionally inactivated or otherwise modulated by epigenetic alterations; for example, alterations in the genome other than the DNA sequence itself, which include genomic hypomethylations, promoter-related hypermnethylation (e.g., of CpG dinucleotides, and CpG islands), histone deacetylation and chromatin modifications. Molecular analysis of tumor-derived genetic and epigenetic alterations may have a profound impact on cancer diagnosis and monitoring for tumor recurrence.

Therefore, there is a need in the art to identify differentially expressed genes (e.g., using s) between cancer and corresponding normal tissues to advance the understanding of the molecular basis of malignancy, and to provide diagnostic and/or prognostic markers of malignancy and methods for using these markers, as well as to provide novel therapeutic targets and corresponding methods of treatment.

There is a need in the art to identify and statistically correlate altered gene expression that is characteristic of the specific stage of the cancer to provide compositions and methods that are independent and/or supplementary to the standard histopathological approaches to work-up of precancerous and cancerous lesions of the prostate.

### SUMMARY OF THE INVENTION

Genes expression was profiled in benign and untreated human prostate cancer tissues using oligonucleotide s. Six hundred seventy-four (674) genes with distinct (i.e., differential expression relative to benign tissue) expression patterns in metastatic and confined tumors (Gleason score 6 and 9, lymph node invasive and non-invasive) were identified. Validation of expression profiles of seventeen (17) genes by quantitative PCR revealed a strong inverse correlation in the expression with progression of prostate cancer for: zinc finger protein (ZNF185), bullous pemphigoid antigen gene (BPAG1), prostate secretory protein (PSP94) (see EXAMPLE I below); and for supervillin (SVIL); proline rich membrane anchor 1 (PRIMA1); TU3A; FLJ14084; KIAA1210; sorbin and SH3 domain containing 1 (SORBS1); and C21orf63 (see EXAMPLE II below.

Likewise, the validated up-regulated genes include: Erg-2, MARCKS-like protein (MLP); SRY (sex determining region Y)-box 4 (SOX4); Fatty acid binding protein 5 (FABP5); and MAL2.

Additionally, the mRNA expression levels of the ZNF185, FLJ14084, SVIL, KIAA1210, PRIMA1 and TU3A genes in prostate cancer cell lines were restored by treatment of cells with 5-aza-2′-deoxycytidine, an inhibitor of DNA methylation, thereby implicating the transcriptional silencing of these genes by methylation in prostate cancer cells, and indicating that genomic DNA methylation is correlated with prostate tumorigenesis.

Methylation-specific PCR even further confirmed methylation of the 5′CpG islands of the ZNF185 gene in all metastatic tissues and 44% of the localized tumor tissues as well as in the prostate cancer cell lines tested. Thus, transcriptional silencing of particular inventive markers, including ZNF185, by DNA methylation in prostate tumor tissues is correlated with prostate tumorigenesis and progression.

Various aspects of the present invention provide one or more gene markers, or panels thereof, whereby at least one of expression, and methylation analysis of one or a combination of the members of the panel enables the detection of cell proliferative disorders of the prostate with a particularly high sensitivity, specificity and/or predictive value. The inventive testing methods have particular utility for the screening of at-risk populations. The inventive methods have advantages over prior art methods, because of improved sensitivity, specificity and likely patient compliance.

The present invention provides novel methods for detecting or distinguishing between prostate cell proliferative disorders.

One embodiment the invention provides a method for detecting and/or for detecting and distinguishing between or among prostate cell proliferative disorders in a subject. Said method comprises: i) contacting genomic DNA isolated from a test sample obtained from the subject with at least one reagent, or series of reagents that distinguishes between methylated and non-methylated CpG dinucleotides within at least one target region of the genomic DNA, wherein the nucleotide sequence of said target region comprises at least one CpG dinucleotide sequence; and ii) detecting, or detecting and distinguishing between or among prostate cell proliferative disorders based on determination of the corresponding genomic methylation state.

Another embodiment the method comprises the use of one or more genes or genomic sequences selected from the group consisting of: (ZNF185), bullous pemphigoid antigen gene (BPAG1), prostate secretory protein (PSP94), supervillin (SVIL); proline rich membrane anchor 1 (PRIMA1); TU3A; FLJ14084; KIAA1210; sorbin and SH3 domain containing 1 (SORBS1), C21orf63, Erg-2, MARCKS-like protein (MLP); SRY (sex determining region Y)-box 4 (SOX4); Fatty acid binding protein 5 (FABP5); and MAL2.as markers for the differentiation, detection and distinguishing of prostate cell proliferative disorders and cancer.

Said use of the gene may be enabled by means of any analysis of the expression of the gene, by means of mRNA expression analysis or protein expression analysis. However, in the most preferred embodiment of the invention, the detection, differentiation and distinguishing of colorectal cell proliferative disorders is enabled by means of analysis of the methylation status of one or more genes or genomic sequences selected from the group consisting of: (ZNF185), bullous pemphigoid antigen gene (BPAG1), prostate secretory protein (PSP94), supervillin (SVIL); proline rich membrane anchor 1 (PRIMA1); TU3A; FLJ14084; KIAA1210; sorbin and SH3 domain containing 1 (SORBS1), C21orf63, Erg-2, MARCKS-like protein (MLP); SRY (sex determining region Y)-box 4 (SOX4); Fatty acid binding protein 5 (FABP5); and MAL2 (and their regulatory and promoter elements) as markers for the differentiation, detection and distinguishing of prostate cell proliferative disorders and cancer.

The present invention provides a method for ascertaining genetic and/or epigenetic parameters of genomic DNA. The method has utility for the improved diagnosis, treatment and monitoring of prostate cell proliferative disorders, more specifically by enabling the improved identification of and differentiation between subclasses of said disorder or stages of prostate tumors.

Preferably, the source of the test sample is selected from the group consisting of cells or cell lines, histological slides, biopsies, paraffin-embedded tissue, bodily fluids, ejaculate, stool, urine, blood, and combinations thereof.

Specifically, the present invention provides a method for detecting prostate cell proliferative disorders, comprising: obtaining a biological sample comprising genomic nucleic acid(s); contacting the nucleic acid(s), or a fragment thereof, with one reagent or a plurality of reagents sufficient for distinguishing between methylated and non methylated CpG dinucleotide sequences within a target sequence of the subject nucleic acid, wherein the target sequence comprises, or hybridizes under stringent conditions to, a sequence comprising at least 16 contiguous nucleotides of SEQ ID NOS:1, 29, 31, 32, 34, 35, 37, 38, 40, 42, 43, 45, 47, 49 and 51, said contiguous nucleotides comprising at least one CpG dinucleotide sequence; and determining, based at least in part on said distinguishing, the methylation state of at least one target CpG dinucleotide sequence, or an average, or a value reflecting an average methylation state of a plurality of target CpG dinucleotide sequences. Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence.

Additional embodiments provide a method for the detection of prostate cell proliferative disorders, comprising: obtaining a biological sample having subject genomic DNA; extracting the genomic DNA; treating the genomic DNA, or a fragment thereof, with one or more reagents to convert 5-position unmethylated cytosine bases to uracil or to another base that is detectably dissimilar to cytosine in terms of hybridization properties; contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a sequence selected from the group consisting of the bisulfite converted sequences corresponding to SEQ ID NOS:1, 29, 31, 32, 34, 35, 37, 38, 40, 42, 43, 45, 47, 49 and 51, wherein the treated DNA or the fragment thereof is either amplified to produce an amplificate, or is not amplified; and determining, based on a presence or absence of, or on a property of said amplificate, the methylation state of at least one CpG dinucleotide sequence selected from the group consisting of SEQ ID NOS:1, 29, 31, 32, 34, 35, 37, 38, 40, 42, 43, 45, 47, 49 and 51, or an average, or a value reflecting an average methylation state of a plurality of CpG dinucleotide sequences thereof. Preferably, at least one such hybridizing nucleic acid molecule or peptide nucleic acid molecule is bound to a solid phase.

Further embodiments provide a method for the analysis of prostate cell proliferative disorders, comprising: obtaining a biological sample having subject genomic DNA; extracting the genomic DNA; contacting the genomic DNA, or a fragment thereof, comprising one or more sequences selected from the group consisting of SEQ ID NOS:1, 29, 31, 32, 34, 35, 37, 38, 40, 42, 43, 45, 47, 49 and 51, or a sequence that hybridizes under stringent conditions thereto, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence of one or more sequences selected from the group consisting of SEQ ID NOS:1, 29, 31, 32, 34, 35, 37, 38, 40, 42, 43, 45, 47, 49 and 51, or an average, or a value reflecting an average methylation state of a plurality of CpG dinucleotide sequences thereof. Preferably, the digested or undigested genomic DNA is amplified prior to said determining.

Additional embodiments provide novel genomic and chemically modified nucleic acid sequences, as well as oligonucleotides and/or PNA-oligomers for analysis of cytosine methylation patterns within sequences from the group consisting of SEQ ID NOS:1, 29, 31, 32, 34, 35, 37, 38, 40, 42, 43, 45, 47, 49 and 51.

### BRIEF DESCRIPTION OF THE DRAWINGS

FIG. 1 shows expression of 50 significantly regulated genes in 36 prostate tissue samples (the text of FIG. 1 is reproduced in TABLE 4). Cluster diagram depicting genes that distinguish metastatic (Met; n=5) from confined tumors with Gleason score 9 lymph node positive (9P; n=6) or negative (9N; n=6) and Gleason score 6 lymph node positive (6P; n=6) or negative (6N; n=5) prostate cancer and adjacent benign tissues (ABT; n=8) (n represents the number of tissues). Each row represents a gene and each column a tissue sample. Red and green represent up regulation and down regulation, respectively, relative to the median of the reference pool. Gray represents technically inadequate or missing date, and black represents equal expression relative to the reference samples. Color saturation is proportional to the magnitude of the difference from the mean. Each gene is labeled by its gene name. Mean and standard deviation (S.D.) of the fold change in the expression levels of genes compared to ABT is shown.

FIG. 2a shows forward primer (FP), reverse primer (RP) and probes used for Taqman real-time PCR.

FIG. 2b shows expression levels of genes ZNF185, PSP94, BPAG1 and Erg-2 as validated by Taqman real-time PCR in 36 samples (28 cancer and 8 benign) used for analysis and an additional 8 samples (4 cancer and 4 benign). Values are expressed as the copy number of the gene relative to GAPDH levels. Metastatic tissues (Met ν) n=5, Gleason score 9, lymph node positive (9P ▪) n=7 or negative (9N □) n=8 and Gleason score 6, lymph node positive (6P λ) n=6 or negative tissues (6N ∘) n=6 and adjacent benign tissues (ABT σ) n=12 were used. (n represents the number of tissues). Mean ± standard deviation (S.D.) of relative expression levels of each group is shown on the left.

FIG. 3a shows expression of ZNF185 levels in prostate cancer cells treated with 6 μM 5-Aza-CdR for 6 days. Four separate experiments are represented, and the error bars denote the standard deviation. The symbol “*” Indicates statistical significance over the untreated cells (p<0.05%).

FIG. 3b shows the PCR primers (forward primer [FP], reverse primer [RP]), used for MSP of prostate tissues. The symbol “W” represents unmodified or wild type primers, “M,” methylated-specific primers, and “U,” unmethylated-specific primers. Sequence difference between modified primers and unmodified DNA are in boldface type and differences between methylated/modified and unmethylated/modified are underlined.

FIG. 3c shows MSP analysis of ZNF185 DNA in prostate tissue samples and cell lines, with and without 5-Aza-CdR treatment. The amplified products were directly loaded onto DNA 500 lab chip and analyzed on Agilent 2100 Bioanalyzer. Molecular size marker is shown at left. All DNA samples were bisulfite-treated except those designated untreated. The experiments were repeated twice and the representative band of the PCR product in lanes U, M and W indicates the presence of unmethylated, methylated and wild type ZNF185 DNA, respectively.

FIG. 3d shows a summary of the incidence of methylation of ZNF185 DNA in prostate tissues analyzed by MSP.

FIGS. 4-14 show, respectively, the expression levels of eleven genes (PRIMA , TU3A, KIAA1210, FLJ14084; SVIL, SORBS1, C21orf63, MAL2, FABP5, SOX4 and MLP) as validated by Taqman real-time PCR analysis (including the Kruskal-Wallis global test) in 40 prostate tissue samples and expressed as the relative fold increase (MAL2, FABP5, SOX4 and MLP) or decrease (PRIMA1, TU3A, KIAA1210, FLJ14084; SVIL, SORBS1 and C21orf63) in the mRNA expression over the adjacent benign tissues after normalization to the house-keeping gene GAPDH mRNA levels. Mean and standard deviations are shown on the right. This real-time PCR data validates results from the instant-based expression analysis. A significant decrease in the expression of the PRIMA1, TU3A, KIAA1210, FLJ14084; SVIL, SORBS1 and C21orf63 genes was confirmed in metastatic versus organ confined and localized tumors compared to benign tissues (p<0.0004), and the MAL2, FABP5, SOX4 and MLP genes were confirmed to be upregulated in the expression in Gleason grade 6 and Gleason grade 9 tissues compared to the metastatic tissues.

FIGS. 15-19 show, respectively, for the FLJ14084, SVIL, PRIMA1, KIAA1210 and TU3A genes, enhanced expression of mRNA levels in prostate cancer cells (LAPC4, LNCaP and PC3 cell lines) treated with 6 μM 5-Aza-CdR for 6 days. Four separate experiments are represented, and the error bars denote the standard deviation. The asterisk (*) indicates statistical significance over the untreated cells (p<0.05%). The increase in the mRNA levels of FLJ14084, SVIL, PRIMA1, KIAA1210 and TU3A by 5-Aza-CdR indicates that the gene is silenced by methylation in prostate cancer cells.

### DETAILED DESCRIPTION OF THE INVENTION

Genes expression was profiled in benign and untreated human prostate cancer tissues using oligonucleotide s. Six hundred seventy-four (674) genes with distinct (i.e., differential expression relative to benign tissue) expression patterns in metastatic and confined tumors (Gleason score 6 and 9, lymph node invasive and non-invasive) were identified. Validation of expression profiles of seventeen (17) genes by quantitative PCR revealed a strong inverse correlation in the expression with progression of prostate cancer for: zinc finger protein (ZNF185), bullous pemphigoid antigen gene (BPAG1), prostate secretory protein (PSP94) (see EXAMPLE I below); and for supervillin (SVIL); proline rich membrane anchor 1 I (PRIMA1); TU3A; FLJ4084; KIAA1210; sorbin and SH43 domain containing 1 (SORBS1); and C21orf63 (see EXAMPLE II below.

Likewise, the validated up-regulated genes include: Erg-2, MARCKS-like protein (MLP); SRY (sex determining region Y)-box 4 (SOX4); Fatty acid binding protein 5 (FABP5); and MAL2.

Additionally, the mRNA expression levels of the ZNF185, FLJ14084, SVIL, KIAA1210, PRIMA1 and TU3A genes in prostate cancer cell lines were restored by treatment of cells with 5-aza-2′-deoxycytidine, an inhibitor of DNA methylation, thereby implicating the transcriptional silencing of these genes by methylation in prostate cancer cells, and indicating that genomic DNA methylation is correlated with prostate tumorigenesis.

Methylation-specific PCR even further confirmed methylation of the 5′CpG islands of the ZNF185 gene in all metastatic tissues and 44% of the localized tumor tissues as well as in the prostate cancer cell lines tested. Thus, transcriptional silencing of particular inventive markers, including ZNF185, by DNA methylation in prostate tumor tissues is correlated with prostate tumorigenesis and progression.

Definitions:

“ZNF185” (SEQ ID NOS:1 and 2) refers to the zinc finger protein 185 nucleic acid sequence (NM_{—}007150; Y09538) and protein, and additionally includes functional variants (including conservative amino acid sequence variants as described herein), fragments, muteins, derivatives and fusion proteins thereof;

“PSP94” (SEQ ID NOS:29 and 30) refers to Prostate secretory protein 94 PSP94 nucleic acid (NM_{—}002443; Homo sapiens microseminoprotein, beta-(MSMB), transcript variant PSP94) and protein, and additionally includes functional variants (including conservative amino acid sequence variants as described herein), fragments, muteins, derivatives and fusion proteins thereof;

“BPAG1” (SEQ ID NO:31) refers to Bullous pemphigoid antigen 1 nucleic acid (HUMBPAG1A; M69225; Human bullous pemphigoid antigen (BPAG1)) and protein, and additionally includes functional variants (including conservative amino acid sequence variants as described herein), fragments, muteins, derivatives and fusion proteins thereof;

“Erg-2” (SEQ ID NOS: 51 and 52) refers to Homo sapiens v-ets erythroblastosis virus E26 oncogene like (avian) (ERG), transcript variant 2 nucleic acid (NM_{—}004449) and protein, and additionally includes functional variants (including conservative amino acid sequence variants as described herein), fragments, muteins, derivatives and fusion proteins thereof;

“SVIL” (SEQ ID NOS:35 and 36) refers to supervillin (SVIL) nucleic acid (AF051851.1; Homo sapiens supervillin) and protein, and additionally includes functional variants (including conservative amino acid sequence variants as described herein), fragments, muteins, derivatives and fusion proteins thereof;

“PRIMA 1” (SEQ ID NO:37) refers to proline rich membrane anchor 1 (PRIMA1) nucleic acid (AI823645) and protein, and additionally includes functional variants (including conservative amino acid sequence variants as described herein), fragments, muteins, derivatives and fusion proteins thereof;

“TU3A” (SEQ ID NOS:40 and 41) refers to Homo sapiens nucleic acid (mRNA; cDNA DKFZp564N0582, from clone DKFZp564N0582) (AL050264) and protein, and additionally includes functional variants (including conservative amino acid sequence variants as described herein), fragments, muteins, derivatives and fusion proteins thereof;

“FLJ14084” (SEQ ID NOS:38 and 39) refers to FLJ14084 nucleic acid (NM_{—}021637) and protein, and additionally includes functional variants (including conservative amino acid sequence variants as described herein), fragments, muteins, derivatives and fusion proteins thereof;

“KIAA1210” (SEQ ID NO:42) refers to the EST corresponding to A1610999;

“SORBS1” (SEQ ID NOS:32 and 33) refers to sorbin and SH3 domain containing 1 (SORBS1) nucleic acid (NM_{—}015385; Homo sapiens sorbin and SH3 domain containing 1 (SORBS1)) and protein, and additionally includes functional variants (including conservative amino acid sequence variants as described herein), fragments, muteins, derivatives and fusion proteins thereof;

“C21orf63” (SEQ ID NO:34)refers to the EST C21ORF63; AI744591;

“MLP” (SEQ ID NOS:45 and 46) refers to Homo sapiens macrophage myristoylated alanine-rich C kinase substrate(MACMARCKS); MARCKS-like protein (MLP) nucleic acid (NM_{—}023009.1) and protein, and additionally includes functional variants (including conservative amino acid sequence variants as described herein), fragments, muteins, derivatives and fusion proteins thereof;

“SOX4” (SEQ ID NOS:43 and 44) refers to Homo sapiens SRY (sex determining region Y)-box 4 (SOX4) nucleic acid (NM_{—}003107) and protein, and additionally includes functional variants (including conservative amino acid sequence variants as described herein), fragments, muteins, derivatives and fusion proteins thereof;

“FABP5” (SEQ ID NOS:47 and 48) refers to Homo sapiens fatty acid binding protein 5 (FABP5) (psoriasis-associated) nucleic acid (NM_{—}001444.1) and protein, and additionally includes functional variants (including conservative amino acid sequence variants as described herein), fragments, muteins, derivatives and fusion proteins thereof;

“MAL2” (SEQ ID NOS:49 and 50) refers to Homo sapiens mal, T-cell differentiation protein 2 (MAL2), or to Homo sapiens MAL2 proteolipid (MAL2) nucleic acid (NM_{—}052886; AY007723) and protein, and additionally includes functional variants (including conservative amino acid sequence variants as described herein), fragments, muteins, derivatives and fusion proteins thereof;

The terms “LNCaP,” “PC3” and “LAPC4” refer to the respective art-recognized human prostate cancer cell lines. Specifically, the human prostate cancer cell lines LNCaP, PC3 are from American Type Culture Collection, Rockville, Md., USA, and LAPC4 was a gift from Dr. Charles L. Sawyers, University of California, Los Angeles, Calif.;

The term “Observed/Expected Ratio” (“O/E Ratio”) refers to the frequency of CpG dinucleotides within a particular DNA sequence, and corresponds to the [number of CpG sites/(number of C bases×number of G bases)]×band length for each fragment;

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb, or to about 2 kb in length;

The term “methylation state” or “methylation status” refers to the presence or absence of 5-methylcytosine (“5-mCyt”) at one or a plurality of CpG dinucleotides within a DNA sequence. Methylation states at one or more particular palindromic CpG methylation sites (each having two CpG CpG dinucleotide sequences) within a DNA sequence include “unmethylated,” “fully-methylated” and “hemi-methylated”;

The term “hemi-methylation” or “hemimethylation” refers to the methylation state of a palindromic CpG methylation site, where only a single cytosine in one of the two CpG dinucleotide sequences of the palindromic CpG methylation site is methylated (e.g., 5′-CC^{M}GG-3′ (top strand): 3′-GGCC-5′ (bottom strand));

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample;

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample;

The term “ ” refers broadly to both “DNAs,” and ‘DNA chip(s),’ as recognized in the art, encompasses all art-recognized solid supports, and encompasses all methods for affixing nucleic acid molecules thereto or synthesis of nucleic acids thereon;

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms);

“Epigenetic parameters” are, in particular, cytosine methylations. Further epigenetic parameters include, for example, the acetylation of histones which, however, cannot be directly analyzed using the described method but which, in turn, correlate with the DNA methylation;

The term “bisulfite reagent” refers to a reagent comprising bisulfite, disulfite, hydrogen sulfite or combinations thereof, useful as disclosed herein to distinguish between methylated and unmethylated CpG dinucleotide sequences;

The term “Methylation assay” refers to any assay for determining the methylation state of one or more CpG dinucleotide sequences within a sequence of DNA;

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997;

The term “MethyLight™” refers to the art-recognized fluorescence-based real-time PCR technique described by Eads et al., Cancer Res. 59:2302-2306, 1999;

The term “HeavyMethyl™” assay, in the embodiment thereof implemented herein, refers to an assay, wherein methylation specific blocking probes (also referred to herein as blockers) covering CpG positions between, or covered by the amplification primers enable methylation-specific selective amplification of a nucleic acid sample;

The term “Ms-SNuPE” (Methylation-sensitive Single Nucleotide Primer Extension) refers to the art-recognized assay described by Gonzalgo & Jones, Nucleic Acids Res. 25:2529-2531, 1997;

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146;

The term “COBRA” (Combined Bisulfite Restriction Analysis) refers to the art-recognized methylation assay described by Xiong & Laird, Nucleic Acids Res. 25:2532-2534, 1997;

The term “MCA” (Methylated CpG Island Amplification) refers to the methylation assay described by Toyota et al., Cancer Res. 59:2307-12, 1999, and in WO 00/26401A1;

The term “hybridization” is to be understood as a bond of an oligonucleotide to a complementary sequence along the lines of the Watson-Crick base pairings in the sample DNA, forming a duplex structure; and

“Stringent hybridization conditions,” as defined herein, involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

A conservative amino acid change, as is known in the relevant art, refers to a substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids. It is reasonable to expect that an isolated replacement of a leucine with an isoleucine or valine, an aspartate with a glutamate, a threonine with a serine, or a similar replacement of an amino acid with a structurally related amino acid will not have an effect on the biological properties of the resulting protein or polypeptide variant.

All references cited herein are thereby incorporated herein in their entirety.

Overview

According to EXAMPLE I below, the present invention provides, inter alia, biologically and clinical relevant clusters of genes characteristic of prostate cancer versus benign tissues and confined versus metastatic prostate cancer using oligonucleotide s. In EXAMPLE I, expression profiles were generated from 5 metastatic prostate tissues, and 23 confined tumors including 12 Gleason score 9 (high grade), and 11 Gleason score 6 (intermediate grade) tumors. In addition, 8 adjacent benign prostatic tissues were also studied. In EXAMPLE I, fifty (50) genes have been identified herein with distinct expression patterns in prostate cancer compared with benign prostatic tissues. Expression levels of prostate secretory protein (PSP94), zinc finger protein (ZNF185), bullous pemphigoid antigen gene (BPAG1), prostate specific transglutaminase gene (TGM4), Erg isoform 2 (Erg-2) and Rho GDP dissociation inhibitor (RhoGD-β) were validated by Taqman quantitative real-time PCR. Furthermore, analysis of the expression of ZNF185 in prostate cancer cell lines revealed an increase in the expression by treatment with an inhibitor of DNA methylation, 5-aza-2′-deoxycytidine. Methylation specific PCR (MSP) indicated ZNF185 inactivation by CpG dinucleotide methylations in prostate cancer cell lines and cancer tissues. Our studies show that down-regulation of ZNF185, PSP94 and BPAG1 with epigenetic alteration of ZNF185 is highly associated with prostate cancer progression and serve as useful biomarkers for predicting progression of the cancer.

Likewise, according to EXAMPLE II below, the present invention provides, inter alia, biologically and clinical relevant clusters of genes characteristic of prostate cancer versus benign tissues and confined versus metastatic prostate cancer using oligonucleotide s. In EXAMPLE II, six hundred-twenty four (624) genes were shown by the analysis to have distinct expression patterns in metastatic and confined tumors (Gleason score 6 and 9, relative to benign tissues. A total of eleven (11) of these differentially expressed genes were selected and further validation by Taqman quantitative real time PCR to confirm the differential expression of genes according to the data.

The validated genes include seven (7) down-regulated genes, and four (4) up-regulated genes. Specifically, the validated down-regulated genes include: Supervillin (SVIL); Proline rich membrane anchor 1 (PRIMA1); TU3A; FLJ14084; KIAA1210; Sorbin and SH3 domain containing 1 (SORBS1); and C21orf63. The validated up-regulated genes include: MARCKS-like protein (MLP); SRY (sex determining region Y)-box 4 (SOX4); Fatty acid binding protein 5 (FABP5); and MAL2.

Validation confirmed the -based strong inverse correlation in the expression of all seven down-regulated genes (SVIL, PRIMA1, TU3A, FLJ14084; KIAA1210, SORBS1 and C21orf63) with progression of prostate cancer.

Likewise, validation confirmed the microarray-based correlation of increased expression, in Gleason grade 6 and Gleason grade 9 tissues, for all four upregulated genes (MLP, SOX4, FABP5 and MAL2).

Furthermore, the mRNA expression levels of the FLJ14084, SVIL, KIAA1210, PRIMA1 and TU3A genes in prostate cancer cell lines were restored by treatment of cells with 5-aza-2′-deoxycytidine, an inhibitor of DNA methylation, thereby implicating the transcriptional silencing of these genes by methylation in prostate cancer cells, and indicating that genomic DNA methylation is correlated with prostate tumorigenesis.

According to aspects of the present invention, the altered methylation and/or expression of these genes provide for novel diagnostic and/or prognostic assays for detection of precancerous and cancerous lesions of the prostate. The inventive compositions and methods have great utility as independent and/or supplementary approaches to standard histopathological work-up of precancerous and cancerous lesions of the prostate.

Oligonucleotides. The present invention provides novel uses for genomic sequences selected from the group consisting of SEQ ID NOS:1, 29, 31, 32, 34, 35, 37, 38, 40, 42, 43, 45, 47, 49 and 51, to the complements thereof, to the bisulfite-converted sequences thereof (see below), and to the complements of the bisulfite-converted sequences thereof. Additional embodiments provide modified variants of SEQ ID NOS:1, 29, 31, 32, 34, 35, 37, 38, 40, 42, 43, 45, 47, 49 and 51, to the complements thereof, to the bisulfite-converted sequences thereof (see below), and to the complements of the bisulfite-converted sequences thereof, as well as oligonucleotides and/or PNA-oligomers for analysis of cytosine methylation patterns within SEQ ID NOS: 1, 29, 31, 32, 34, 35, 37, 38, 40, 42, 43, 45, 47, 49 and 51, to the complements thereof, to the bisulfite-converted sequences thereof(see below), and to the complements of the bisulfite-converted sequences thereof.

An objective of the invention comprises analysis of the methylation state of one or more CpG dinucleotides within at least one of the genomic sequences selected from the group consisting of SEQ ID NOS:1, 29, 31, 32, 34, 35, 37, 38, 40, 42, 43, 45, 47, 49 and 51, to the complements thereof, to the bisulfite-converted sequences thereof (see below), and to the complements of the bisulfite-converted sequences thereof.

The disclosed invention provides treated nucleic acids, derived from genomic SEQ ID NOS:1, 29, 31, 32, 34, 35, 37, 38, 40, 42, 43, 45, 47, 49 and 51, and from the complements thereof, wherein the treatment is suitable to convert at least one unmethylated cytosine base of the genomic DNA sequence to uracil or another base that is detectably dissimilar to cytosine in terms of hybridization. The genomic sequences in question may comprise one, or more, consecutive or random methylated CpG positions. Said treatment preferably comprises use of a reagent selected from the group consisting of bisulfite, hydrogen sulfite, disulfite, and combinations thereof. In a preferred embodiment of the invention, the objective comprises analysis of a modified nucleic acid comprising a sequence of at least 16, at least 18, at least 20, at least 25, or at least 30 contiguous nucleotide bases in length of a sequence selected from the group consisting of SEQ ID NOS:1, 29, 31, 32, 34, 35, 37, 38, 40, 42, 43, 45, 47, 49 and 51, the complements thereof, the bisulfite-converted sequences thereof (see below), and the complements of the bisulfite-converted sequences thereof, wherein said sequence comprises at least one CpG, TpA or CpA dinucleotide and sequences complementary thereto. The sequences of the modified versions of the nucleic acid according to SEQ ID NOS:1, 29, 31, 32, 34, 35, 37, 38, 40, 42, 43, 45, 47, 49 and 51, the complements thereof, are encompassed, wherein the modification of each genomic sequence results in the synthesis of a nucleic acid having a sequence that is unique and distinct from said genomic sequence as follows. For each sense strand genomic DNA, e.g., SEQ ID NO:1, four converted versions are disclosed. A first version wherein “C”→“T,” but “CpG” remains “CpG” (i.e., corresponds to case where, for the genomic sequence, all “C” residues of CpG dinucleotide sequences are methylated and are thus not converted); a second version discloses the complement of the disclosed genomic DNA sequence (i.e. antisense strand), wherein “C”→“T,” but “CpG” remains “CpG” (i.e., corresponds to case where, for all “C” residues of CpG dinucleotide sequences are methylated and are thus not converted). The ‘upmethylated’ converted sequences of SEQ ID NOS:1, 29, 31, 32, 34, 35, 37, 38, 40, 42, 43, 45, 47, 49 and 51, and the complements thereof are encompassed herein. A third chemically converted version of each genomic sequences is provided, wherein “C”→“T” for all “C” residues, including those of “CpG” dinucleotide sequences (i.e., corresponds to case where, for the genomic sequences, all “C” residues of CpG dinucleotide sequences are unmethylated); a final chemically converted version of each sequence, discloses the complement of the disclosed genomic DNA sequence (i.e. antisense strand), wherein “C”→“T” for all “C” residues, including those of “CpG” dinucleotide sequences (i.e., corresponds to case where, for the complement (antisense strand) of each genomic sequence, all “C” residues of CpG dinucleotide sequences are unmethylated). The ‘downmethylated’ converted sequences of SEQ ID NOS:1, 29, 31, 32, 34, 35, 37, 38, 40, 42, 43, 45, 47, 49 and 51, and of the complements thereof are additionally encompassed herein.

In an alternative preferred embodiment, such analysis comprises the use of an oligonucleotide or oligomer for detecting the cytosine methylation state within genomic or pretreated (chemically modified) DNA, corresponding to SEQ ID NOS:1, 29, 31, 32, 34, 35, 37, 38, 40, 42, 43, 45, 47, 49 and 51, and to the complements thereof. Said oligonucleotide or oligomer comprising a nucleic acid sequence having a length of at least 9, at least 15, at least 18, at least 20, at least 25, or at least 30 nucleotides which hybridizes, under moderately stringent or stringent conditions (as defined herein above), to a pretreated nucleic acid sequence, or to a genomic sequence according to SEQ ID NOS:1, 29, 31, 32, 34, 35, 37, 38, 40, 42, 43, 45, 47, 49 and 51, or to the complements thereof.

The present invention includes nucleic acid molecules (e.g., oligonucleotides and peptide nucleic acid (PNA) molecules (PNA-oligomers)) that hybridize under moderately stringent and/or stringent hybridization conditions to all or a portion of the sequences SEQ ID NOS:1, 29, 31, 32, 34, 35, 37, 38, 40, 42, 43, 45, 47, 49 and 51, to the complements thereof, to the bisulfite-converted sequences thereof(see below), and to the complements of the bisulfite-converted sequences thereof. The hybridizing portion of the hybridizing nucleic acids is typically at least 9, 15, 20, 25, 30 or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NOS:1, 29, 31, 32, 34, 35, 37, 38, 40, 42, 43, 45, 47, 49 and 51, to the complements thereof, to the bisulfite-converted sequences thereof (see below), and to the complements of the bisulfite-converted sequences thereof.

Hybridizing nucleic acids of the type described herein can be used, for example, as a primer (e.g., a PCR primer), or a diagnostic and/or prognostic probe or primer. Preferably, hybridization of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO:1 (and the other SEQ ID NOS recited above) (such as allelic variants and SNPs), rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to SEQ ID NO:1, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:

n to (n+(X−1));

where n=1, 2, 3, . . . (Y−(X−1));

where Y equals the length (nucleotides or base pairs) of SEQ ID NO:1 (3,614);

where X equals the common length (in nucleotides) of each oligonucleotide in the set (e.g., X=20 for a set of consecutively overlapping 20-mers); and

where the number (Z) of consecutively overlapping oligomers of length X for a given SEQ ID NO of length Y is equal to Y−(X−1). For example Z=3,614−19=3,595 for either sense or antisense sets of SEQ ID NO:1, where X=20.

Preferably, the set is limited to those oligomers that comprise at least one CpG, TpG or CpA dinucleotide.

Examples of inventive 20-mer oligonucleotides include the following set of 3,595 oligomers (and the antisense set complementary thereto), indicated by polynucleotide positions with reference to SEQ ID NO:1:

1-20, 2-21, 3-22, 4-23, 5-24 . . . 3593-3612, 3594-3613 and 3595-3614.

Preferably, the set is limited to those oligomers that comprise at least one CpG, TpG or CpA dinucleotide.

The present invention encompasses, for SEQ ID NO:1 (sense and antisense), multiple consecutively overlapping sets of oligonucleotides or modified oligonucleotides of length X, where, e.g., X=9, 10, 17, 20, 22, 23, 25, 27, 30 or 35 nucleotides. Likewise, the invention encompasses analogous sets of oligos corresponding to SEQ ID NOS:1, 29, 31, 32, 34, 35, 37, 38, 40, 42, 43, 45, 47, 49 and 51, to the complements thereof, to the bisulfite-converted sequences thereof(see below), and to the complements of the bisulfite-converted sequences thereof.

The oligonucleotides or oligomers according to the present invention constitute effective tools useful to ascertain genetic and epigenetic parameters of the genomic sequence corresponding to SEQ ID NOS:1, 29, 31, 32, 34, 35, 37, 38, 40, 42, 43, 45, 47, 49 and 51, to the complements thereof, to the bisulfite-converted sequences thereof (see below), and to the complements of the bisulfite-converted sequences thereof. Preferred sets of such oligonucleotides or modified oligonucleotides of length X are those consecutively overlapping sets of oligomers corresponding to at least one of SEQ ID NOS:1, 29, 31, 32, 34, 35, 37, 38, 40, 42, 43, 45, 47, 49 and 51, to the complements thereof, to the bisulfite-converted sequences thereof (see below), and to the complements of the bisulfite-converted sequences thereof. Preferably, said oligomers comprise at least one CpG, TpG or CpA dinucleotide.

Oligonucleotides and PNA-oligomers capable of hybridizing, as described herein above, to the various bisulfite-converted sequences of SEQ ID NOS:1, 29, 31, 32, 34, 35, 37, 38, 40, 42, 43, 45, 47, 49 and 51, and to the complements of the bisulfite-converted sequences thereof are also within the scope of the present invention.

The oligonucleotides of the invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. No. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc.

The oligonucleotide may also comprise at least one art-recognized modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of each of the CpG dinucleotides of genomic sequences SEQ ID NOS:1, 29, 31, 32, 34, 35, 37, 38, 40, 42, 43, 45, 47, 49 and 51, to the complements thereof, or to the corresponding CpG, TpG or CpA dinucleotide within a sequence of the corresponding pretreated nucleic acids, and sequences complementary thereto. However, it is anticipated that for economic or other factors it may be preferable to analyze a limited selection of the CpG dinucleotides within said sequences, and the content of the set of oligonucleotides is altered accordingly.

Therefore, in particular embodiments, the present invention provides a set of at least two (2) (oligonucleotides and/or PNA-oligomers) useful for detecting the cytosine methylation state in pretreated genomic DNA corresponding to SEQ ID NOS:1, 29, 31, 32, 34, 35, 37, 38, 40, 42, 43, 45, 47, 49 and 51, to the complements thereof. These probes enable diagnosis, classification and/or therapy of genetic and epigenetic parameters of prostate cell proliferative disorders and tumors. The set of oligomers may also be used for detecting single nucleotide polymorphisms (SNPs) in the above-described pretreated genomic DNA, and sequences complementary thereto.

In preferred embodiments, at least one, and more preferably all members of a set of oligonucleotides is bound to a solid phase.

In further embodiments, the present invention provides a set of at least two (2) oligonucleotides that are used as ‘primer’ oligonucleotides for amplifying DNA sequences of one of SEQ ID NOS:1, 29, 31, 32, 34, 35, 37, 38, 40, 42, 43, 45, 47, 49 and 51, the complements thereof, the bisulfite-converted sequences thereof (see below), or the complements of the bisulfite-converted sequences thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may be composed of silicon, glass, polystyrene, aluminum, steel, iron, copper, nickel, silver, or gold. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example, via a confocal microscope. Cy3 and Cy5 dyes, besides many others, are commercially available.

It is also anticipated that the oligonucleotides, or particular sequences thereof, may constitute all or part of an “virtual array” wherein the oligonucleotides, or particular sequences thereof, are used, for example, as ‘specifiers’ as part of, or in combination with a diverse population of unique labeled probes to analyze a complex mixture of analytes. Such a method, for example is described in US 2003/0013091 (U.S. Ser. No. 09/898,743, published 16 Jan. 2003). In such methods, enough labels are generated so that each nucleic acid in the complex mixture (i.e., each analyte) can be uniquely bound by a unique label and thus detected (each label is directly counted, resulting in a digital read-out of each molecular species in the mixture).

It is particularly preferred that the oligomers according to the invention are utilised for at least one of: detection of; detection and differentiation between or among subclasses of; diagnosis of; prognosis of; treatment of; monitoring of; and treatment and monitoring of prostate cell proliferative disorders and cancer. This is enabled by use of said sets for the detection or detection and differentiation of one or more prostate tissues as described herein.

In preferred embodiments, expression or genomic methylation state is determined by one or more methods comprising amplification of ‘treated’ (e.g., bisulfite-treated) DNA. The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labeled amplificates have a single positive or negative net charge, allowing for better detectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionately with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

Methylation Assay Procedures. Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, and use of methylation-sensitive restriction enzymes.

For example, genomic sequencing has been simplified for analysis of DNA methylation patterns and 5-methylcytosine distribution by using bisulfite treatment (Frommer et al., Proc. Natl. Acad. Sci. USA 89:1827-1831,1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

COBRA. COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the interested CpG islands, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Preferably, assays such as “MethyLight™” (a fluorescence-based real-time PCR technique) (Eads et al., Cancer Res. 59:2302-2306,1999), Ms-SNuPE (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad Sci. USA 93:9821-9826,1996; U.S. Pat. No. 5,786,146), and methylated CpG island amplification (“MCA”; Toyota et al., Cancer Res. 59:2307-12, 1999) are used alone or in combination with other of these methods.

MethyLight™. The MethyLight™ assay is a high-throughput quantitative methylation assay that utilizes fluorescence-based real-time PCR (TaqMan™) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight™ assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight™ process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan(& probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific gene (or methylation-altered DNA sequence or CpG island); TaqMan® probes; optimized PCR buffers and deoxynucleotides; and Taq polymerase.

Ms-SNuPE. The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

MSP. MSP (methylation-specific PCR) allows for assessing the methylation status of virtually any group of CpG sites within a CpG island, independent of the use of methylation-sensitive restriction enzymes (Herman et al. Proc. Natl. Acad Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

MCA. The MCA technique is a method that can be used to screen for altered methylation patterns in genomic DNA, and to isolate specific sequences associated with these changes (Toyota et al., Cancer Res. 59:2307-12, 1999). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

### Preferred Embodiments

Particular aspects of the present invention provide a method for detecting, or for detecting and distinguishing between or among prostate cell proliferative disorders or stages thereof in a subject comprising:obtaining, from the subject, a biological sample; and determining, using a suitable assay, the expression level of at least one gene or sequence selected from the group consisting of: ZNF185 (SEQ ID NOS:1 and 2); PSP94 (SEQ ID NOS:29 and 30); BPAG1 (SEQ ID NO:31); SORBS1 (SEQ ID NOS:32 and 33); C21orf63 (SEQ ID NO:34); SVIL (SEQ ID NOS:35 and 36); PRIMA1 (SEQ ID NO:37); FLJ14084 (SEQ ID NOS:38 and 39); TU3A (SEQ ID NOS:40 and 41); KIAA1210 (SEQ ID NO:42); SOX4 (SEQ ID NOS:43 and 44); MLP (SEQ ID NOS:45 and 46); FABP5 (SEQ ID NOS:47 and 48); MAL2 (SEQ ID NOS:49 and 50); Erg-2 (SEQ ID NOS: 51 and 52); and sequences that hybridize under high stringency thereto, whereby detecting and distinguishing between or among prostate cell proliferative disorders or stages thereof is, at least in part, afforded.

Preferably, the expression level is determined by detecting the presence, absence or level of mRNA transcribed from said gene or sequence. Preferably, the expression level is determined by detecting the presence, absence or level of a polypeptide encoded by said gene or sequence. Preferably, the polypeptide is detected by at least one method selected from the group consisting of immunoassay, ELISA immunoassay, radioimmunoassay, and antibody. Preferably, the expression is determined by detecting the presence or absence of CpG methylation within said gene or sequence, wherein hypermethylation indicates the presence of, or stage of the prostate cell proliferative disorder.

Preferably, detecting and distinguishing between or among prostate cell proliferative disorders or stages thereof is, at least in part, based on a decrease in expression of at least one gene or sequence selected from the group consisting of: ZNF185 (SEQ ID NOS:1 and 2); PSP94 (SEQ ID NOS:29 and 30); BPAG1 (SEQ ID NO:31); SORBS1 (SEQ ID NOS:32 and 33); C21orf63 (SEQ ID NO:34); SVIL (SEQ ID NOS:35 and 36); PRIMA1 (SEQ ID NO:37); FLJ14084 (SEQ ID NOS:38 and 39); TU3A (SEQ ID NOS:40 and 41); KIAA1210 (SEQ ID NO:42); and sequences that hybridize under high stringency thereto. Preferably, and alternatively, detecting and distinguishing between or among prostate cell proliferative disorders or stages thereof is, at least in part, based on a increase in expression of at least one gene or sequence selected from the group consisting of: SOX4 (SEQ ID NOS:43 and 44); MLP (SEQ ID NOS:45 and 46); FABP5 (SEQ ID NOS:47 and 48); MAL2 (SEQ ID NOS:49 and 50); Erg-2 (SEQ ID NOS: 51 and 52); and sequences that hybridize under high stringency thereto.

Preferably, expression is of at least one gene or sequence selected from the group consisting of: ZNF185 (SEQ ID NOS:1 and 2); SVIL (SEQ ID NOS:35 and 36); PRIMA1 (SEQ ID NO:37); FLJ14084 (SEQ ID NOS:38 and 39); TU3A (SEQ ID NOS:40 and 41); KIAA1210 (SEQ ID NO:42); and sequences that hybridize under high stringency thereto.

Additional embodiments provide a method for detecting, or for detecting and distinguishing between or among prostate cell proliferative disorders or stages thereof in a subject, comprising: obtaining, from the subject, a biological sample having genomic DNA; and contacting genomic DNA obtained from the subject with at least one reagent, or series of reagents that distinguishes between methylated and non-methylated CpG dinucleotides within at least one target region of the genomic DNA, wherein the target region comprises, or hybridizes under stringent conditions to at least 16 contiguous nucleotides of at least one sequence selected from the group consisting of SEQ ID NOS:1, 29, 31, 32, 34, 35, 37, 38, 40, 42, 43, 45, 47, 49, 51, and complements thereof, wherein said contiguous nucleotides comprise at least one CpG dinucleotide sequence, and whereby detecting, or detecting and distinguishing between or among colon cell proliferative disorders or stages thereof is, at least in part, afforded.

Preferably, normal, non-prostate cell proliferative disorders, or adjacent benign tissues are distinguished from at least one condition selected from the group consisting of: intermediate, T2, Gleason score 6 lymph node positive and negative; high grade, T3, Gleason score 9 lymph node positive and negative; prostatic adenocarcinoma; and metastatic tumors.

Preferably, adjacent benign tissue is distinguished from at least one condition selected from the group consisting of: intermediate, T2, Gleason score 6 lymph node positive and negative; high grade, T3, Gleason score 9 lymph node positive and negative; prostatic adenocarcinoma; and metastatic tumors. Preferably, adjacent benign tissue is distinguished from at least one condition selected from the group consisting of: intermediate, T2, Gleason score 6 lymph node positive and negative; high grade, T3, Gleason score 9 lymph node positive and negative; prostatic adenocarcinoma; and metastatic tumors, and the target region comprises, or hybridizes under stringent conditions to at least 16 contiguous nucleotides of a sequence selected from the group consisting of ZNF185 (SEQ ID NO:1); PSP94 (SEQ ID NO:29); BPAG1 (SEQ ID NO:31); SORBS1 (SEQ ID NO:32); C21orf63 (SEQ ID NO:34); SVIL (SEQ ID NS:35); PRIMA1 (SEQ ID NO:37); FLJ14084 (SEQ ID NO:38); TU3A (SEQ ID NO:40); KIAA1210 (SEQ ID NO:42); and sequences complementary thereto. Preferably, adjacent benign tissue is distinguished from at least one condition selected from the group consisting of: intermediate, T2, Gleason score 6 lymph node positive and negative; high grade, T3, Gleason score 9 lymph node positive and negative; prostatic adenocarcinoma; and metastatic tumors, and the target region comprises, or hybridizes under stringent conditions to at least 16 contiguous nucleotides of a sequence selected from the group consisting of ZNF185 (SEQ ID NO:1); SVIL (SEQ ID NO:35); PRIMA1 (SEQ ID NO:37); FLJ14084 (SEQ ID NO:38); TU3A (SEQ ID NO:40); KIAA1210 (SEQ ID NO:42); and sequences complementary thereto.

In alternate preferred embodiments, tissues originating from the prostate are distinguished from tissues of non-prostate origin. Preferably, prostate cell proliferative disorders are distinguished from healthy tissues, and the target region comprises, or hybridizes under stringent conditions to at least 16 contiguous nucleotides of a sequence selected from the group consisting of ZNF185 (SEQ ID NO:1); PSP94 (SEQ ID NO:29); BPAG1 (SEQ ID NO:31); SORBS1 (SEQ ID NO:32); C21orf63 (SEQ ID NO:34); SVIL (SEQ ID NO:35); PRIMA1 (SEQ ID NO:37); FLJ14084 (SEQ ID NO:38); TU3A (SEQ ID NO:40); KIAA1210 (SEQ ID NO:42); and sequences complementary thereto.

Yet further embodiments provide a method for detecting, or for detecting and distinguishing between or among prostate cell proliferative disorders or stages thereof in a subject, comprising: obtaining, from a subject, a biological sample having genomic DNA; contacting the genomic DNA, or a fragment thereof, with one reagent or a plurality of reagents that distinguishes between methylated and non methylated CpG dinucleotide sequences within at least one target sequence of the genomic DNA, or fragment thereof, wherein the target sequence comprises, or hybridizes under stringent conditions to, at least 16 contiguous nucleotides of a sequence taken from the group consisting of SEQ ID NOS:1, 29, 31, 32, 34, 35, 37, 38, 40, 42, 43, 45, 47, 49, 51, and complements thereof, said contiguous nucleotides comprising at least one CpG dinucleotide sequence; and determining, based at least in part on said distinguishing, the methylation state of at least one target CpG dinucleotide sequence, or an average, or a value reflecting an average methylation state of a plurality of target CpG dinucleotide sequences, whereby detecting, or detecting and distinguishing between or among prostate cell proliferative disorders or stages thereof is, at least in part, afforded.

Preferably, detecting, or detecting and distinguishing between or among prostate cell proliferative disorders or stages thereof comprises detecting, or detecting and distinguishing between or among one or more tissues selected from the group consisting of: adjacent benign tissues; intermediate, T2, Gleason score 6 lymph node positive or negative tissue; high grade, T3, Gleason score 9 lymph node positive or negative tissue; prostatic adenocarcinoma; and metastatic tumors.

Preferably, distinguishing between methylated and non methylated CpG dinucleotide sequences within the target sequence comprises converting unmethylated cytosine bases within the target sequence to uracil or to another base that is detectably dissimilar to cytosine in terms of hybridization properties. Preferably, distinguishing between methylated and non methylated CpG dinucleotide sequences within the target sequence(s) comprises methylation state-dependent conversion or non-conversion of at least one CpG dinucleotide sequence to the corresponding converted or non-converted dinucleotide sequence.

Preferably, the biological sample is selected from the group consisting of cell lines, histological slides, biopsies, paraffin-embedded tissue, bodily fluids, ejaculate, urine, blood, and combinations thereof.

Preferably, distinguishing between methylated and non methylated CpG dinucleotide sequences within the target sequence comprises use of at least one nucleic acid molecule or peptide nucleic acid (PNA) molecule comprising, in each case a contiguous sequence at least 9 nucleotides in length that is complementary to, or hybridizes under stringent conditions to a bisulfite-converted sequence derived from a sequence selected from the group consisting of SEQ ID NOS: 1, 29, 31, 32, 34, 35, 37, 38, 40, 42, 43, 45, 47, 49, 51, and complements thereof. Preferably, the contiguous sequence comprises at least one CpG, TpG or CpA dinucleotide sequence. Preferably, at least two such nucleic acid molecules, or peptide nucleic acid (PNA) molecules are used. Preferably, at least two such nucleic acid molecules are used as primer oligonucleotides for the amplification of a bisulfite-converted sequence derived from a sequence selected from the group consisting of SEQ ID NOS:1, 29, 31, 32, 34, 35, 37, 38, 40, 42, 43, 45, 47, 49, 51; sequences that hybridize under stringent conditions therto; and complements thereof. Preferably, at least four such nucleic acid molecules, peptide nucleic acid (PNA) molecules are used.

Further embodiments provide a method for detecting, or detecting and distinguishing between or among prostate cell proliferative disorders or stages thereof in a subject, comprising: obtaining, from a subject, a biological sample having genomic DNA; extracting or otherwise isolating the genomic DNA; treating the genomic DNA, or a fragment thereof, with one or more reagents to convert cytosine bases that are unmethylated in the 5-position thereof to uracil or to another base that is detectably dissimilar to cytosine in terms of hybridization properties; contacting the treated genomic DNA, or the treated fragment thereof, with an amplification enzyme and at least two primers comprising, in each case a contiguous sequence of at least 9 nucleotides that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45,47, 49, 51, and complements thereof, wherein the treated genomic DNA or the fragment thereof is either amplified to produce at least one amplificate, or is not amplified; and determining, based on a presence or absence of, or on a property of said amplificate, the methylation state of at least one CpG dinucleotide of a sequence selected from the group consisting of SEQ ID NOS:1, 29, 31, 32, 34, 35, 37, 38, 40, 42, 43, 45, 47, 49, 51, and complements thereof, or an average, or a value reflecting an average methylation state of a plurality of said CpG dinucleotides, whereby at least one of detecting, and detecting and distinguishing between prostate cell proliferative disorders or stages thereof is, at least in part, afforded.

Preferably, treating the genomic DNA, or the fragment thereof comprises use of a reagent selected from the group consisting of bisulfite, hydrogen sulfite, disulfite, and combinations thereof. Preferably, contacting or amplifying comprises use of at least one method selected from the group consisting of: use of a heat-resistant DNA polymerase as the amplification enzyme; use of a polymerase lacking 5′-3′ exonuclease activity; use of a polymerase chain reaction (PCR); generation of a amplificate nucleic acid molecule carrying a detectable labels; and combinations thereof.

Preferably, the detectable amplificate label is selected from the label group consisting of: fluorescent labels; radionuclides or radiolabels; amplificate mass labels detectable in a mass spectrometer; detachable amplificate fragment mass labels detectable in a mass spectrometer; amplificate, and detachable amplificate fragment mass labels having a single-positive or single-negative net charge detectable in a mass spectrometer; and combinations thereof.

Preferably, the biological sample obtained from the subject is selected from the group consisting of cell lines, histological slides, biopsies, paraffin-embedded tissue, bodily fluids, ejaculate, urine, blood, and combinations thereof.

Preferably, detecting, or detecting and distinguishing between or among prostate cell proliferative disorders or stages thereof comprises detecting, or detecting and distinguishing between or among one or more tissues selected from the group consisting of: adjacent benign tissues; intermediate, T2, Gleason score 6 lymph node positive or negative tissue; high grade, T3, Gleason score 9 lymph node positive or negative tissue; prostatic adenocarcinoma; and metastatic tumors.

Preferably, the method further comprises, for the step of contacting the treated genomic DNA, the use of at least one nucleic acid molecule or peptide nucleic acid molecule comprising in each case a contiguous sequence at least 9 nucleotides in length that is complementary to, or hybridizes under stringent conditions to a bisulfite-converted sequence derived from a sequence selected from the group consisting of SEQ ID NOS: 1, 29, 31, 32, 34, 35, 37, 38, 40, 42, 43, 45, 47, 49, 51, and complements thereof, wherein said nucleic acid molecule or peptide nucleic acid molecule suppresses amplification of the nucleic acid to which it is hybridized.

Preferably, the nucleic acid molecule or peptide nucleic acid molecule is in each case modified at the 5′-end thereof to preclude degradation by an enzyme having 5′-3′ exonuclease activity. Preferably, the nucleic acid molecule or peptide nucleic acid molecule is in each case lacking a 3′ hydroxyl group. Preferably, the amplification enzyme is a polymerase lacking 5′-3′ exonuclease activity.

Preferably, “determining” comprises hybridization of at least one nucleic acid molecule or peptide nucleic acid molecule in each case comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof. Preferably, at least one such hybridizing nucleic acid molecule or peptide nucleic acid molecule is bound to a solid phase. Preferably, a plurality of such hybridizing nucleic acid molecules or peptide nucleic acid molecules are bound to a solid phase in the form of a nucleic acid or peptide nucleic acid array selected from the array group consisting of linear or substantially so, hexagonal or substantially so, rectangular or substantially so, and combinations thereof.

Preferably, the method further comprises extending at least one such hybridized nucleic acid molecule by at least one nucleotide base. Preferably, “determining” comprises sequencing of the amplificate. Preferably, “contacting” or amplifying comprises use of methylation-specific primers.

Preferably, for the “contacting” step, primer oligonucleotides comprising one or more CpG; TpG or CpA dinucleotidesn are used; and the method further comprises, for the determining step, the use of at least one method selected from the group consisting of: hybridizing in at least one nucleic acid molecule or peptide nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof; hybridizing at least one nucleic acid molecule that is bound to a solid phase and comprises a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof; hybridizing at least one nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, and extending at least one such hybridized nucleic acid molecule by at least one nucleotide base; and sequencing, in the determining step, of the amplificate.

Preferably, for the contacting step, uat least one nucleic acid molecule or peptide nucleic acid molecule is used, comprising in each case a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, wherein said nucleic acid molecule or peptide nucleic acid molecule suppresses amplification of the nucleic acid to which it is hybridized; and the method further comprises, in the determining step, the use of at least one method selected from the group consisting of: hybridizing in at least one nucleic acid molecule or peptide nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof; hybridizing at least one nucleic acid molecule that is bound to a solid phase and comprises a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof; hybridizing at least one nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, and extending at least one such hybridized nucleic acid molecule by at least one nucleotide base; and sequencing, in the determining step, of the amplificate.

Preferably, the method comprises, in the “contacting” step, amplification by primer oligonucleotides comprising one or more CpG; TpG or CpA dinucleotides, and further comprises, in the “determining” step, hybridizing at least one detectably labeled nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof.

Preferably, the method comprises, in the “contacting” step, the use of at least one nucleic acid molecule or peptide nucleic acid molecule comprising in each case a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, wherein said nucleic acid molecule or peptide nucleic acid molecule suppresses amplification of the nucleic acid to which it is hybridized, and further comprises, in the “determining” step, hybridizing at least one detectably labeled nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof.

Yet additional embodiments provide a method for detecting, or for detecting and distinguishing between or among prostate cell proliferative disorders or stages thereof in a subject, comprising: obtaining, from a subject, a biological sample having genomic DNA; extracting, or otherwise isolating the genomic DNA; contacting the genomic DNA, or a fragment thereof, comprising at least 16 contiguous nucleotides of a sequence selected from the group consisting of SEQ ID NOS:1, 29, 31, 32, 34, 35, 37, 38, 40, 42, 43, 45, 47, 49, 51, complements thereof; and sequences that hybridize under stringent conditions thereto, with one or more methylation-sensitive restriction enzymes, wherein the genomic DNA is, with respect to each cleavage recognition motif thereof, either cleaved thereby to produce cleavage fragments, or not cleaved thereby; and determining, based on a presence or absence of, or on property of at least one such cleavage fragment, the methylation state of at least one CpG dinucleotide of a sequence selected from the group consisting of SEQ ID NOS:1, 29, 31, 32, 34, 35, 37, 38, 40, 42, 43, 45, 47, 49, 51; and complements thereof, or an average, or a value reflecting an average methylation state of a plurality of said CpG dinucleotides, whereby at least one of detecting, or of detecting and differentiating between or among prostate cell proliferative disorders or stages thereof is, at least in part, afforded.

Preferably, the method further comprises, prior to determining, amplifying of the digested or undigested genomic DNA. Preferably, amplifying comprises use of at least one method selected from the group consisting of: use of a heat resistant DNA polymerase as an amplification enzyme; use of a polymerase lacking 5′-3′ exonuclease activity; use of a polymerase chain reaction (PCR); generation of a amplificate nucleic acid carrying a detectable label; and combinations thereof.

Preferalby, the detectable amplificate label is selected from the label group consisting of: fluorescent labels; radionuclides or radiolabels; amplificate mass labels detectable in a mass spectrometer; detachable amplificate fragment mass labels detectable in a mass spectrometer; amplificate, and detachable amplificate fragment mass labels having a single-positive or single-negative net charge detectable in a mass spectrometer; and combinations thereof.

Preferably, the biological sample obtained from the subject is selected from the group consisting of cell lines, histological slides, biopsies, paraffin-embedded tissue, bodily fluids, ejaculate, urine, blood, and combinations thereof.

Further embodiments provide an isolated treated nucleic acid derived from SEQ ID NOS:1, 29, 31, 32, 34, 35, 37, 38, 40, 42, 43, 45, 47, 49, 51, and complements thereof, wherein the treatment is suitable to convert at least one unmethylated cytosine base of the genomic DNA sequence to uracil or another base that is detectably dissimilar to cytosine in terms of hybridization.

Additional embodiments provide a nucleic acid, comprising at least 16 contiguous nucleotides of a treated genomic DNA sequence derived from a sequence selected from the group consisting of SEQ ID NOS:1, 29, 31, 32, 34, 35, 37, 38, 40, 42, 43, 45, 47, 49, 51, and complements thereof, wherein the treatment is suitable to convert at least one unmethylated cytosine base of the genomic DNA sequence to uracil or another base that is detectably dissimilar to cytosine in terms of hybridization. Preferably, the contiguous base sequence comprises at least one CpG, TpG or CpA dinucleotide sequence. Preferbly, the treatment comprises use of a reagent selected from the group consisting of bisulfite, hydrogen sulfite, disulfite, and combinations thereof.

Yet additional embodiments provide an oligomer, comprising a sequence of at least 9 contiguous nucleotides that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof. Preferably, the oligomer comprises at least one CpG, CpA or TpG dinucleotide sequence.

Also provided is a set of oligomers, comprising at least two oligonucleotides according, in each case, to those described above.

Preferred embodiments provide a novel use of a set of oligonucleotides as disclosed herein for at least one of: detection of; detection and differentiation between or among subclasses or stages of; diagnosis of; prognosis of; treatment of; monitoring of; and treatment and monitoring of prostate cell proliferative disorders.

Additional preferred aspects provide use of the disclosed inventive nucleic acids, the disclosed inventive oligomers, or a disclosed set of inventive oligonucleotides for detecting, or detecting and distinguishing between or among prostate cell proliferative disorders or stages thereof selected from the group consisting of: adjacent benign tissues; intermediate, T2, Gleason score 6 lymph node positive or negative tissue; high grade, T3, Gleason score 9 lymph node positive or negative tissue; prostatic adenocarcinoma; and metastatic tumors.

Alternate embodiments provide for use of a set of inventive oligomers as probes for determining at least one of a cytosine methylation state, and a single nucleotide polymorphism (SNP) of a sequence selected from the group consisting of SEQ ID NOS:1, 29, 31, 32, 34, 35, 37, 38, 40, 42, 43, 45, 47, 49, 51, and sequences complementary thereto. Preferably, at least two inventive oligomers are used as primer oligonucleotides for the amplification of a DNA sequence of at least 16 contiguous nucleotides of a bisulfite-converted sequence derived from a sequence selected from the group consisting of SEQ ID NOS:1, 29, 31, 32, 34, 35, 37, 38, 40, 42, 43, 45, 47, 49, 51, and complements thereof.

Also disclosed and provided is the use of an inventive nucleic acid for determination of at least one of cytosine methylation status of a corresponding genomic DNA, or detection of a single nucleotide polymorphism (SNP).

Additional embodiments provide a method for manufacturing a nucleic acid array, comprising at least one of attachment of an inventive oligomer, or attachment of a set of such oligomers or nucleic acids, to a solid phase. Further embodiments provide an oligomer array manufactured as described herein. Preferably, the oligomers are bound to a planar solid phase in the form of a lattice selected from the group consisting of linear or substantially linear lattice, hexagonal or substantially hexagonal lattice, rectangular or substantially rectangular lattice, and lattice combinations thereof. In preferred embodiments, the oligomer arrays are used for the analysis of prostate cell proliferative disorders. Preferably, the solid phase surface comprises a material selected from the group consisting of silicon, glass, polystyrene, aluminum, steel, iron, copper, nickel, silver, gold, and combinations thereof.

Yet further embodiments provide a kit useful for detecting, or for detecting and distinguishing between or among prostate cell proliferative disorders or stages thereof of a subject, comprising: at least one of a bisulfite reagent, and a methylation-sensitive restriction enzyme; and at least one nucleic acid molecule or peptide nucleic acid molecule comprising, in each case a contiguous sequence at least 9 nucleotides that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof. Preferably, the kit further comprises standard reagents for performing a methylation assay selected from the group consisting of MS-SNuPE, MSP, MethyLight, HeavyMethyl, COBRA, nucleic acid sequencing, and combinations thereof. Preferably, the above described methods comprise use of the kit according to claim 68.

Additional embodiments provide for use of: an inventive nucleic acid, an inventive oligomer, a set of inventive oligomers, a method of array manufacturing as described herein, an inventive array, and an inventive kit for the detection of, detection and differentiation between or among subclasses or stages of, diagnosis of, prognosis of, treatment of, monitoring of, or treatment and monitoring of prostate cell proliferative disorders.

Pharmaceutical Compositions and Therapeutic Uses

Pharmaceutical compositions of the invention can protein and protein-based agents of the claimed invention in a therapeutically effective amount. The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/ kg to 50 mg/kg or 0.05 mg/kg to about 10 mg/kg of the protein or polypeptide constructs in the individual to which it is administered. A non-limiting example of a pharmaceutical composition is a composition that either enhances or diminishes signaling mediated by a target receptor. Where such signaling promotes a disease-related process, modulation of the signaling would be the goal of the therapy.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., New Jersey, 1991).

Delivery Methods. Once formulated, the compositions of the invention can be administered directly to the subject or delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, myocardial, intratumoral, peritumoral, or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, direct microinjection of the DNA into nuclei, and viral-mediated, such as adenovirus or alphavirus, all well known in the art.

In a preferred embodiment, disorders of proliferation, such as cancer, can be amenable to treatment by administration of a therapeutic agent based on the provided polynucleotide or corresponding polypeptide. The therapeutic agent can be administered in conjunction with one or more other agents including, but not limited to, receptor-specific antibodies and/or chemotherapeutic agents. Administered “in conjunction” includes administration at the same time, or within 1 day, 12 hours, 6 hours, one hour, or less than one hour, as the other therapeutic agent(s). The compositions may be mixed for co-administration, or may be administered separately by the same or different routes.

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic compositions agents of the invention includes local or systemic administration, including injection, oral administration, particle gun or catheterized administration, and topical administration. The therapeutic polynucleotide composition can contain an expression construct comprising a promoter operably linked to a polynucleotide encoding, for example, about 80 to 419 (or about 350 to 419) contiguous amino acids of SEQ ID NO:2. Various methods can be used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. X-ray imaging is used to assist in certain of the above delivery methods.

Protein-, or polypeptide-mediated targeted delivery of therapeutic agents to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1 994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 mg to about 2 mg, about 5 mg to about 500 mg, and about 20 mg to about 100 mg of DNA can also be used during a gene therapy protocol. Factors such as method of action (e.g., for enhancing or inhibiting levels of the encoded gene product) and efficacy of transformation and expression are considerations which will affect the dosage required for ultimate efficacy of the subgenomic polynucleotides. Where greater expression is desired over a larger area of tissue, larger amounts of subgenomic polynucleotides or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect. Gene Therapy. The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. 264:16985 (1989)); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. 14:2411 (1994), and in Woffendin, Proc. Natl. Acad. Sci. (1994) 91:11581-11585.

Further non-viral delivery suitable for use includes mechanical delivery systems such as the approach described in Woffendin et al., Proc. Natl. Acad Sci. USA 91(24): 11581 (1994). Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

The present invention will now be illustrated by reference to the following examples which set forth particularly advantageous embodiments. However, it should be noted that these embodiments are illustrative and are not to be construed as restricting the invention in any way.

### EXAMPLE 1

### (A Set of Genes was Identified that Characterize Prostate Cancer and Benign Prostatic Tissues)

Materials and Methods

Prostate tissues. Prostate cancer tissue specimens were obtained from patients who had undergone radical prostatectomy for prostate cancer at Mayo Clinic. The Institutional Review Board of Mayo Foundation approved collection of tissues, and their use for this study. None of the patients included in this study had received preoperative hormonal therapy, chemotherapy, or radiotherapy. Harvested tissues were embedded in OCT and frozen at −80° C. until use. A hematoxylin and eosin stained section was prepared to insure that tumor was present in the tissue used for the analyses. Out of 340 tissues available in our tissue bank, we selected tissues that had more than 80% of the neoplastic cells by histological examination. In order to examine differential gene expression in intermediate (Gleason score 6), high grade (Gleason score 9) prostatic adenocarcinoma and metastatic tumors, we studied 11 primary stage T2 Gleason score 6 cancers (six with positive regional lymph nodes and five with negative lymph nodes), 12 primary stage T3 Gleason score 9 cancers (six with positive regional lymph nodes, six with negative lymph nodes), and five metastatic tumors.

TABLE 1 shows Gleason grade, age, pre-operative serum prostate-specific antigen levels and staging of all patients from whom prostate tissues were obtained for this study. Twelve separately collected prostatic tissue samples matched with the cancer tissues (obtained from the same patients) were used as normal controls.

TABLE 1
Prostate tissue samples with preoperative PSA values at diagnosis,
Gleason histological scores, and metastasis status of the tissues.

| Gleason | | | | | |
|---|---|---|---|---|---|
| grade/Lymph | | | Preop PSA | | Metastatic |
| node | Sample ID | Age | (ng/ml) | TNM (97) | site |
| 6/Negative | 6N 1 | 55 | 9.4 | T2b, N0− | |
| | 6N 2 | 50 | 7.5 | T2b, N0− | |
| | 6N 3 | 57 | 10.3 | T2b, N0− | |
| | 6N 4 | 67 | 16.7 | T2b, N0− | |
| | 6N 5 | 68 | 8.1 | T2a, N0− | |
| 6/Positive | 6P 1 | 71 | 17.1 | T2b, N0+ | |
| | 6P 2 | 61 | 5.2 | T2b, N0+ | |
| | 6P 3 | 71 | 41.0 | T2b, N0+ | |
| | 6P 4 | 65 | 7.0 | T2a, N0+ | |
| | 6P 5 | 51 | 14.3 | T2b, N0+ | |
| | 6P 6 | 66 | 23.5 | T2b, N0+ | |
| 9/Negative | 9N 1 | 67 | 21.6 | T3a, N0− | |
| | 9N 2 | 65 | 29.4 | T3b, N0− | |
| | 9N 3 | 65 | 24.9 | T3b, N0− | |
| | 9N 4 | 54 | 50.0 | T3b, N0− | |
| | 9N 5 | 59 | 25.8 | T3b, N0− | |
| | 9N 6 | 71 | 6.1 | T3b, N0− | |
| 9/Positive | 9P 1 | 66 | 4.5 | T3a, N0+ | |
| | 9P 2 | 65 | 6.69 | T3b, N0+ | |
| | 9P 3 | 76 | 7.6 | T3b, N1+ | |
| | 9P 4 | 71 | 467.0 | T3b, N0+ | |
| | 9P 5 | 69 | 5.6 | T3b, N0+ | |
| | 9P 6 | 66 | 2.9 | T3b, N1− | |
| Metastatic | Met 1 | 62 | 0.15 | | Liver |
| | Met 2 | 72 | 97.3 | | Peritoneum |
| | Met 3 | 49 | 0.15 | | Lymph node |
| | Met 4 | 60 | 18.4 | | Lymph node |
| | Met 5 | 68 | 8.9 | | Lung |

Isolation of RNA and gene expression profiling. Thirty prostate tissue sections of 15-μm thicknesses were cut with a cryostat and used for RNA isolation. Total RNA was extracted from frozen tissue sections with Trizol® reagent (Life Technologies, Inc., Carlsbad, Calif.). DNA was removed by treatment of the samples with DNase I using DNA-free™ kit (Ambion, Austin, Tex.) and further RNA cleanup was performed using RNeasy Mini kit (Qiagen, Valencia, Calif.) according to the manufacturer's protocols. RNA quality was monitored by agarose gel electrophoresis and also on Agilent 2100 Bioanalyzer (Agilent Technologies, Palo Alto, Calif.). High-density oligonucleotide s HG-U95Av2 containing 12,625 sequences of human genes and ESTs (Affymetrix, Santa Clara, Calif.) were used in this study. Complementary RNA was prepared, labeled and hybridized to oligonucleotide arrays as described previously (Giordano et al., Am. J. Pathol. 159: 1231-1238, 2001). The arrays were scanned with gene array scanner (Agilent Technologies, Palo Alto, Calif.). All arrays were scaled to a target intensity of 1500. Raw data was collected and analyzed by using Affymetrix Suite 5.0 version.

Quantitative Real-Time RT-PCR. To confirm the differential expression of genes from data, four down-regulated genes, ZNF185, PSP94, BPAG1 and TGM4 and two up-regulated genes Erg-2 and RhoGDI-β were selected for validation by Taqman real-time RT-PCR in a total of 44 tissues, including 36 samples used for s with an additional 4 primary tumors and 4 adjacent benign tissues. One (1) μg of the total RNA was used for first-strand cDNA synthesis. The PCR mix contained 1× reaction buffer (10 mM Tris, 50 mM KCl, pH 8.3), MgCl₂ (5 mM), PCR nucleotide mix (1 mM), random primers (0.08 A260 units), RNase inhibitor (50 units), AMV reverse transcriptase (20 units) in a final volume of 20 μl.

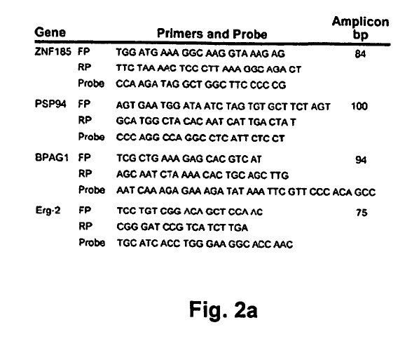

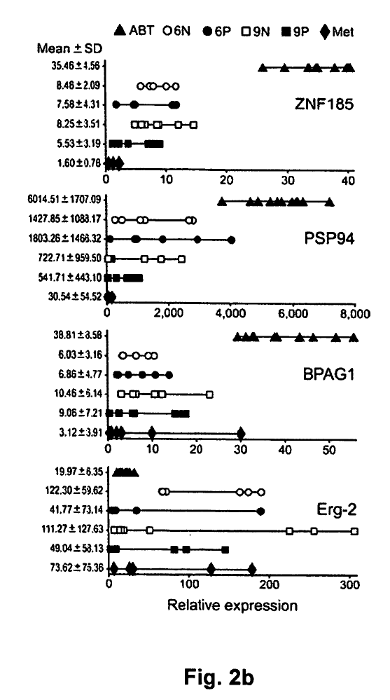

For real-time PCR one μl of the cDNA was used in the PCR reactions. Taqman real-time primers and probes were designed using the software Primer Express™ version 1.5 (PE Applied Biosystems, Foster City, Calif.) and synthesized at Integrated DNA Technologies (Coralville, Iowa). The sequences of the primers and probes for each gene are provided in TABLE 2 and FIG. 2(a).

TABLE 2
Sequences of the primers and probes.

| | | | |
|---|---|---|---|
| | | Amplicon | SEQ ID |
| Gene | Primers and Probe | bp | NO. |

| | | | | |
|---|---|---|---|---|
| ZNF185 | FP | TGGATGAAAGGCAAGGTAAAGAG | 84 | 3 |
| | RP | TTCTAAAACTCCCTTAAAGGCAGACT | | 4 |
| | Probe | CCAAGATAGGCTGGCTTCCCCCG | | 5 |
| PSP94 | FP | AGTGAATGGATAATCTAGTGTGCTTCTAGT | 100 | 6 |
| | RP | GCATGGCTACACAATCATTGACTAT | | 7 |
| | Probe | CCCAGGCCAGGCCTCATTCTCCT | | 8 |
| BPAG1 | FP | TCGCTGAAAGAGCACGTCAT | 94 | 9 |
| | RP | AGCAATCTAAAACACTGCAGCTTG | | 10 |
| | Probe | AATCAAAGAGAAAGATATAAATTCGTTCCCACAGCC | | 11 |
| Erg-2 | FP | TCCTGTCGGACAGCTCCAAC | 75 | 12 |
| | RP | CGGGATCCGTCATCTTGA | | 13 |
| | Probe | TGCATCACCTGGGAAGGCACCAAC | | 14 |

Probes were labeled at 5′ end with the reporter dye 6-carboxyfluorescein (6′-FAM) and at 3′ end with a Black Hole Quencher (BHQ). Probes were purified by reverse phase HPLC and primers were PAGE purified. All PCR reactions were carried out in Taqman Universal PCR master mix (PE Applied Biosytems) with 300 nM of each primer and 200 nM of probe in a final volume of 50 μl. Thermal cycling conditions were as follows: 2 min at 50° C., with denaturation at 95° C. for 10 min, 40 cycles of 15 sec at 95° C. (melting) and 1 min at 60° C. (annealing and elongation). The reactions were performed in an ABI Prism® 7700 Sequence Detection System (PE Applied Biosystems). To evaluate the validity and sensitivity of real-time quantitative PCR, serial dilutions of the oligonucleotide amplicon of the gene in a range of 1 to 1×10⁹ copies were used as corresponding standard. Standard curves were generated using the Cₜ values determined in the real-time PCR to permit gene quantification using the supplied software according to the manufacturer's instructions. In addition, a standard curve was generated for the housekeeping gene, glyceraldehyde-3-phosphate-dehydrogenase (Applied Biosystems, part number 402869) to enable normalization of each gene. Data were expressed as relative copy number of transcripts after normalization.

Cell Lines and 5-Aza-CdR Treatment. The human prostate cancer cell lines LNCaP, PC3 (American Type Culture Collection, Rockville, Md., USA) and LAPC4 (a gift from Dr. Charles L. Sawyers, University of California, Los Angeles, Calif.) were grown in Roswell Park Memorial Institute (RPM1) 1640 medium supplemented with 5% fetal bovine serum (FBS) at 37° C. and 5% CO₂ until reaching approximately 50-70% confluence. Cells were then treated with 5% FBS RPMI 1640 containing 6 μM 5-aza-2′-deoxycytidine (5-Aza-CdR) (Sigma Chemicals Co., St. Louis, Mo.) for 6 days, with medium changes on day 1, 3, and 5. Total RNA was isolated from the cell lines and the expression of the ZNF185 was analyzed by Taqman real-time PCR as described above. The housekeeping gene GAPDH was used as an internal control to enable normalization.

DNA isolation and Bisulfite modification. Genomic DNA was obtained from metastatic, primary, matched benign prostatic tissues and the above mentioned prostate cancer cell lines treated with 5-Aza-CdR, using Wizard® genomic DNA purification kit according to the manufacturer's protocol (Promega, Madison, Wis.). Genomic DNA (100 ng) was modified by sodium bisulfite treatment by converting unmethylated, but not methylated, cytosines to uracil as described previously (Herman et al., Proc. Natl. Acad. Sci. USA 93:9821-9826, 1996). DNA samples were then purified using the spin columns (Qiagen), and eluted in 50 μl of distilled water. Modification was completed by treatment with NaOH (0.3 M final concentration) for 5 min at room temperature, followed by ethanol precipitation. DNA was re-suspended in water and used for PCR amplification.

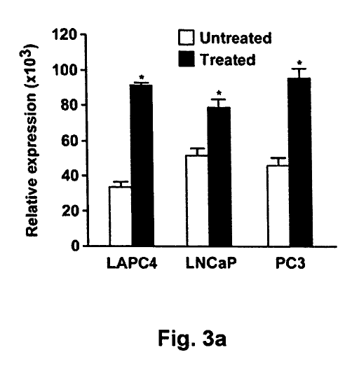

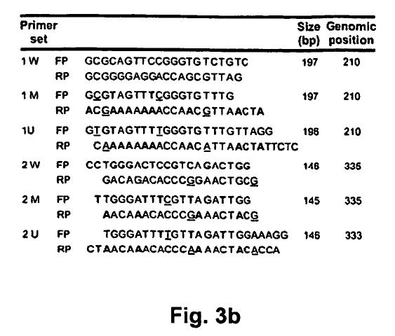

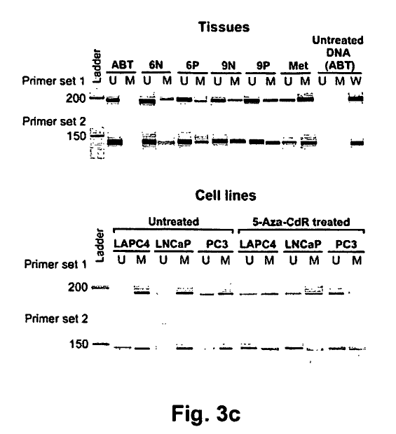

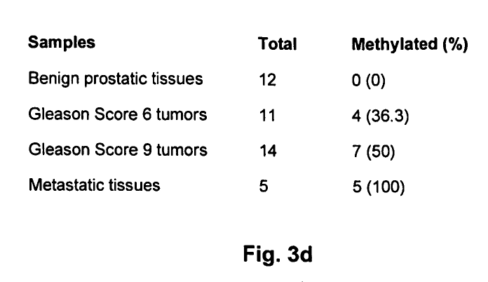

Methylation Specific PCR (MSP). DNA methylation patterns within the gene were determined by chemical modification of unmethylated cytosine to uracil and subsequent PCR as described previously (Esteller et al., Cancer Res. 61:3225-3229, 2001), using primers specific for either methylated or the modified unmethylated sequences. The primers used for MSP were shown in TABLE 3 and FIG. 3(b).

TABLE 3
Primers used for MSP analysis.

| Primer | | | Size | Genomic | SEQ ID |
|---|---|---|---|---|---|
| set | | | bp | position | NO. |
| 1 W | FP | GCGCAGTTCCGGGTGTCTGTC | 197 | 210 | 15 |
| | RP | GCGGGGAGGACCAGCGTTAG | | | 16 |
| 1 M | FP | GCGTAGTTTCGGGTGTTTG | 197 | 210 | 17 |
| | RP | ACGAAAAAAACCAACGTTAACTA | | | 18 |
| 1 U | FP | GTGTAGTTTTGGGTGTTTGTTAGG | 196 | 210 | 19 |
| | RP | CAAAAAAAACCAACATTAACTATTCTC | | | 20 |
| 2 W | FP | CCTGGGACTCCGTCAGACTGG | 146 | 335 | 21 |
| | RP | GACAGACACCCGGAACTGCG | | | 22 |
| 2 M | FP | TTGGGATTTCGTTAGATTGG | 145 | 335 | 23 |
| | RP | AACAAACACCCGAAACTACG | | | 24 |
| 2 U | FP | TGGGATTTTGTTAGATTGGAAAGG | 146 | 333 | 25 |
| | RP | CTAACAAACACCCAAAACTACACCA | | | 26 |

Two sets of primers were designed corresponding to the genomic positions around 210 and 335. Genomic position indicates the location of the 5′ nucleotide of the sense primer in relation to the major transcriptional start site defined in the Genbank accession number (Y09538). The PCR mixture contained 1×PCR buffer (50 mM KCl, 10 mM Tris-HCl pH 8.3 with 0.01% w/v gelatin), dNTPs (0.2 mM each), primers (500 μM) and bisulfite modified or unmodified DNA (100 ng) in a final volume of 25 μl. Reactions were hot-started at 95° C. for 10 min with the addition of 1.25 units of AmpliTaq Gold™ DNA polymerase (PerkinElmer). Amplifications were carried out in GeneAmp PCR systems 9700 (Applied Biosystems) for 35 cycles (30 sec at 95° C., 30 sec at 55° C. and 30 sec at 72° C.), followed by a final 7 min extension at 72° C. Appropriate negative and positive controls were included in each PCR reaction. One (1) μl of the PCR product was directly loaded onto DNA 500 lab chip and analyzed on Agilent 2100 Bioanalyzer (Agilent Technologies, Palo Alto, Calif.).

Results

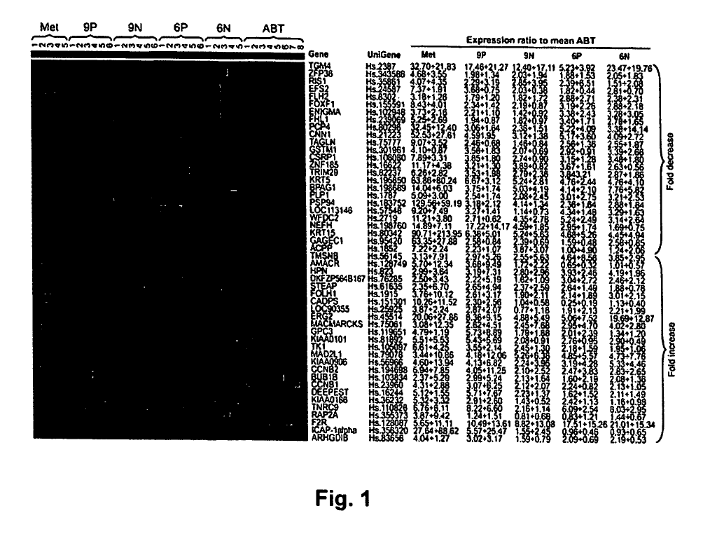

Gene expression profiles of 28 prostate cancer tissues were monitored using oligonucleotide s. A gene-by-gene analysis of the difference in mean log expression between the two groups was performed to identify genes differentially expressed between cancer and benign tissues. Genes were ranked according to inter-sample variability (SD), and 1850 genes with the most variable expression across all of the samples were median-centered and normalized with respect to other genes in the samples and corresponding genes in the other samples. Genes and samples were subjected to hierarchical clustering essentially as described previously (Eisen et al., Proc. Natl. Acad. Sci. USA 95:14863-14868, 1998). Differential expression of genes in benign and malignant prostate tissues was estimated using an algorithm (Giordano et al., Am. J. Pathol. 159:1231-1238, 2001) based on equally weighted contributions from the difference of hybridization intensities (μTumor-μNormal) or (μNormal-μTumor), the quotient of hybridization intensities (μTumor/μNormal) or (μNormal/μTumor), and the result of an unpaired t-test between expression levels in tumor and normal tissues. The selection criteria was narrowed to genes that showed a fold change of >2.35 between normal and cancer samples and a p<0.00 1 by student's t-test. A cluster of 25 up-regulated and 25 down-regulated genes, which discriminated between normal and cancer tissues was identified (FIG. 1).

Among the 25 down-regulated genes identified (FIG. 1), PSP94, BPAG1, WFDC2, KRT5, KRT15, TAGLN, ZFP36 and the genes encoding LIM domain proteins FLH1, FLH2, ENIGMA are consistent with the expression profiles of previous studies (Dhanasekaran et al., Nature 412:822-826,2001; Ernst et al., Am. J. Pathol. 160:2169-2180, 2002; LaTulippe et al., Cancer Res. 62:44994506, 2002; Luo et al., Mol. Carcinog. 33:25-35, 2002; Shields et al., J. Biol. Chem. 277:9790-9799, 2002). Up-regulation of hepsin, AMACR, STEAP, FOLH1, RAP2A and the unknown gene DKFZP564B167 are consistent with the previously published data of analysis (Dhanasekaran et al., supra; Luo et al., Cancer Res. 61:4683-4688, 2001; Magee et al., Cancer Res. 61:5692-5696, 2001; Welsh et al., Cancer Res. 61:5974-5978, 2001; Rubin et al., Journal of the American Medical Assn. 287:1662-1670, 2002; Ernst et al., supra; Luo et al., supra; Rhodes et al., Cancer Res. 62:4427-4433, 2002; Stamey et al., J. Urol. 166:2171-2177, 2001). In addition, the present data also confirms up-regulation of the cell cycle regulated genes CCNB1, CCNB2, MAD2L1, DEEPEST, BUB1B, cell adhesion regulator MACMARCKS, and unclassified genes KIAA0186 and KIAA0906 (Welsh et al., supra; Ernst et al., supra; LaTulippe et al., supra; Stamey et al., supra).

PSP94, ZNF185, BPAG1, and TGM4 were selected from the 25 down-regulated genes and Erg-2 and RhoGDI-β from the 25 up-regulated genes for further validation by Taqman quantitative PCR. These genes were selected because of their moderate to high level expression in prostate cancer. In addition, their potential functions, as mentioned below, are relevant to prostate cancer biology. Furthermore, except for PSP94, their role in prostate cancer biology has not been previously described. PSP94 has been shown to be down-regulated in prostate cancer (Sakai et al., Prostate 38:278-284, 1999) and is the most down-regulated gene in the instant data.

To validate the expression profiles, Taqman quantitative PCR was performed in duplicate for each sample. The standard curve slope values for all the genes ranged between −3.58 and −3.20, corresponding to PCR efficiency of above 0.9. The Kruskal-Wallis global test was done with the real time quantitative analysis for all the genes. A significant decrease in the expression of ZNF185, BPAG1 and PSP94 mRNA levels was observed in metastatic versus organ confined and localized tumors compared to benign tissues [p<0.0001] (FIG. 2b). Moreover, the Wilcoxon test was used to compare each tissue type to the adjacent benign tissues. ZNF185, BPAG1 and PSP94 showed p-values less than 0.0019 in each group compared to benign tissues.

PSP94 is a highly prostate specific gene encoding a major prostate secretory protein. Earlier studies reported that both the secretion and synthesis of PSP94 were reduced in prostate cancer tissues (Sakai et al., supra). PSP94 is involved in inhibition of tumor growth by apoptosis (Garde et al., Prostate 38:118-125, 1999) and the down-regulation in prostate tumor tissues may be the survival mechanism for cancer cells. The instant experiments indicate that PSP94 palys a role in prostate cancer progression.

BPAG1 is a 230-kDa hemi-desmosomal component involved in adherence of epithelial cells to the basement membrane. Previous studies have shown a loss of BPAG1 in invasive breast cancer cells(Bergstraesser et al., Am. J. Pathol. 147:1823-1839,1995). The down-regulation of BPAG1 in our study (>14 fold in metastatic tissues) provides an indicator of an invasive phenotype and predicts the potential of invasive cells to metastasize (Herold-Mende et al., Cell Tissue Res. 306:399-408, 2001).

Erg-2 is a proto-oncogene known to play an important role in the development of cancer (Simpson et al., Oncogene 14:2149-2157, 1997). Erg-2 expression levels were herein observed to increased in 16 (50%) out of 32 cancer tissues when stringently compared to the highest level of Erg-2 in 12 adjacent benign tissues. The increase in mRNA levels of Erg-2 in at least half of the cancer tissues examined indicates a role of Erg-2 in prostate cancer.

Furthermore, TGM4 is a prostate tissue specific transglutaminase (type IV) that has been implicated in apoptosis and cell growth (Antonyak et al., J. Biol. Chem. 278:15859-15866, 2003). RhoGDI-β may be involved in cellular transformation (Lozano et al., Bioessays 25:452-463, 2003). The present Taqman PCR study shows that TGM4 and RhoGDI-β levels were not changed significantly in most of the prostate cancer tissues (data not shown).

ZNF185 is a novel LIM domain gene (Heiss et al., Genomics 43:329-338, 1997), and, according to the present invention, plays a role in prostate cancer development and progression. Particular LIM domain proteins have been shown to play an important role in regulation of cellular proliferation and differentiation (Bach, I., Mech Dev. 91:5-17, 2000; McLoughlin, et al., J. Biol. Chem. 277:37045-37053, 2002; Mousses et al., Cancer Res. 62: 1256-1260, 2002; Yamada et al., Oncogene, 21:1309-1315,2002; Robert et al., Nat. Genet. 33:61-65, 2003). ZNF185 is located on chromosome Xq28, a chromosomal region of interest as a result of the more than 20 hereditary diseases mapped to this region. The ZNF185 LIM is a cysteine-rich motif that coordinately binds two zinc atoms and mediates protein-protein interactions. Heiss et al. (Heiss et al., supra) cloned a full-length ZNF185 cDNA and showed that the transcript is expressed in a very limited number of human tissues with most abundant expression in the prostate.

Significantly, the present invention is the first identification of a correlation of ZNF185 regulation and cancer. Specifically, there was a significant down-regulation in the expression of ZNF185 gene in all prostate cancer tissues compared to benign prostatic tissues (FIGS. 1 and 2b). The decrease in ZNF185 expression in prostate tumors indicated that ZNF185 plays an important role in the development and progression of prostate cancer.

To study the transcriptional silencing of ZNF185 in prostate cancer, LAPC4, LNCaP and PC3 prostate cancer cell lines were treated with 5-Aza-CdR an inhibitor of DNA methyl transferase DNMT1 (Robert et al., supra). Treatment with 5-Aza-CdR showed approximately a 2.0-fold increase in mRNA levels of ZNF185 (FIG. 3a, indicating that the gene might be partially silenced by methylation. To confirm the transcriptional inactivation, MSP was carried out to assess the methylation status of cytosine residues in the 5′ CpG dinucleotides of genomic DNA in prostate tumors, adjacent benign tissues and in prostate cell lines with or without treatment with 5-Aza-CdR. Cytosine methylations within CpG dinucleotides were observed in the prostate cancer tissues and cell lines with two sets of primers used for PCR (FIG. 3c). A reduction of the methylated band and increase of the unmethylated band in cell lines with 5-Aza-CdR treatment is consistent with the restoration of ZNF185 mRNA levels after demethylation. (FIG. 3a).

In most of tissues samples, DNA not treated with bisulfite (unmodified) failed to amplify with either set of methylated or unmethylated specific primers but readily amplified with primers specific for the sequence before modification, suggesting an almost complete bisulfite reaction. Methylation of ZNF185 was accompanied by amplification of the unmethylated reaction as well. The presence of the unmethylated ZNF185 DNA could indicate the presence of normal tissues in these non-microdissected samples. However, heterogeneity in the patterns of methylation in the tumor itself might also be present. Fisher's unordered test for methylation difference in metastatic, confined tumors and benign tissues was highly significant (p<0.0003).

The incidence of methylation in cancer tissues is shown in FIG. 3(d). Methylation status and down-regulation in the mRNA expression is correlated with higher tumor grade and metastasis.

These results indicate that methylation of CpG dinucleotides may be the major factor causing transcriptional inactivation of ZNF185 and repressing its expression in the prostate cancer tissues.

In summary, mRNA expression analysis with oligonucleotide s identified a set of genes that characterize prostate cancer and benign prostatic tissues. A decrease in the expression of genes PSP94, BPAG1 and ZNF185 highly correlates with prostate cancer progression. Increase of Erg-2 levels also indicates its role in development of prostate cancer.

Significantly, this is the first study to identify inactivation of the LIM domain gene ZNF185 in patients with prostate cancer and in prostate cancer cell lines. The present invention identifies this gene as a marker of prostate cancer aggressiveness. According to the present invention, transcriptional silencing of PSP94 and BPAG1 additionally serves as prognostic markers for prostate cancer progression, and as potential therapeutic targets for prostate cancer.

TABLE 1
Prostate tissue samples with preoperative PSA values at diagnosis,
Gleason histological scores, and metastasis status of the tissues.

| Gleason | | | | | |
|---|---|---|---|---|---|
| grade/Lymph | | | Preop PSA | | Metastatic |
| node | Sample ID | Age | (ng/ml) | TNM (97) | site |
| 6/Negative | 6N 1 | 55 | 9.4 | T2b, N0− | |
| | 6N 2 | 50 | 7.5 | T2b, N0− | |
| | 6N 3 | 57 | 10.3 | T2b, N0− | |
| | 6N 4 | 67 | 16.7 | T2b, N0− | |
| | 6N 5 | 68 | 8.1 | T2a, N0− | |
| 6/Positive | 6P 1 | 71 | 17.1 | T2b, N1+ | |
| | 6P 2 | 61 | 5.2 | T2b, N0+ | |
| | 6P 3 | 71 | 41.0 | T2b, N0+ | |
| | 6P 4 | 65 | 7.0 | T2a, N0+ | |
| | 6P 5 | 51 | 14.3 | T2b, N0+ | |
| | 6P 6 | 66 | 23.5 | T2b, N0+ | |
| 9/Negative | 9N 1 | 67 | 21.6 | T3a, N0− | |
| | 9N 2 | 65 | 29.4 | T3b, N0− | |
| | 9N 3 | 65 | 24.9 | T3b, N0− | |
| | 9N 4 | 54 | 50.0 | T3b, N0− | |
| | 9N 5 | 59 | 25.8 | T3b, N0− | |
| | 9N 6 | 71 | 6.1 | T3b, N0− | |
| 9/Positive | 9P 1 | 66 | 4.5 | T3a, N0+ | |
| | 9P 2 | 65 | 6.69 | T3b, N0+ | |
| | 9P 3 | 76 | 7.6 | T3b, N1+ | |
| | 9P 4 | 71 | 467.0 | T3b, N0+ | |
| | 9P 5 | 69 | 5.6 | T3b, N0+ | |
| | 9P 6 | 66 | 2.9 | T3b, N1− | |
| Metastatic | Met 1 | 62 | 0.15 | | Liver |
| | Met 2 | 72 | 97.3 | | Peritoneum |
| | Met 3 | 49 | 0.15 | | Lymph node |
| | Met 4 | 60 | 18.4 | | Lymph node |
| | Met 5 | 68 | 8.9 | | Lung |

### EXAMPLE II

### 624 Genes were Identified by Expression Profiling as having Differential Expression Patterns in Metastatic and Confined Prostate Tumors Relative to Benign Tissues, Eleven (11) of these Genes were Further Validated as Diagnostic/Prognostic Markers by Quantitative Real Time PCR Validation, and 5 Genes were Shown to be Silenced, at Least in Part, by DNA Methylation

In this Example, the expression of genes in benign and untreated human prostate cancer tissues was profiled using oliginucleotide s (Affymetrix U133A and U133B chips). Six hundred-twenty four (624) genes were shown by the analysis to have distinct expression patterns in metastatic and confined tumors (Gleason score 6 and 9, relative to benign tissues. A total of eleven (11) of these differentially expressed genes were selected and further validation by Taqman quantitative real time PCR to confirm the differential expression of genes according to the data.

Materials and Methods:

Prostate Tissue. Prostate cancer tissue specimens were obtained from patients who had undergone radical prostatectomy for prostate cancer at Mayo Clinic as described earlier (Vanaja et al., Cancer Res. 63:3877-3822, 2003).

TABLE 1 (herein below) shows Gleason grade, age, pre-operative serum prostate-specific antigen (PSA) levels at diagnosis, and staging (Gleason histological scores) of all patients from whom prostate tissues were obtained for this study. A total of 40 prostate tissues were used to study the gene expression profiling.

Isolation of RNA and Gene expression profiling. Thirty prostate tissue sections of 15-μm thicknesses were cut with a cryostat and used for RNA isolation. Total RNA was extracted from frozen tissue sections with Trizol® reagent (Life Technologies, Inc., Carlsbad, Calif.). High-density oligonucleotide s, U133A and U133B, containing 44792 sequences of human genes and ESTs (Affymetrix, Santa Clara, Calif.) were used in this study. Complementary RNA was prepared, labeled and hybridized to oligonucleotide arrays as described previously (Vanaja et al., supra).

The expression profiles were generated from 5 metastatic prostate tissues, and 27 confined tumors, including fifteen (15) Gleason score-9 (high grade) and twelve (12) Gleason score-6 (intermediate grade) tumors. Additionally, eight (8) adjacent benign prostatic tissues were also studied. Six hundred forty-two (642) genes with distinct (differential) expression patterns in prostate cancer compared with benign prostatic tissues were identified (see Table 2 herein below).

TABLE 2 shows the differential expression (relative to benign tissue) of 624 significantly regulated genes in 40 prostate tissue samples. The expression is computed as the average of the probes within each probe set of a gene in the chips. The 624 genes were ‘extracted’ from the metastatic vs. benign tissues with significant p-value <0.01. The genes from the combined set of probes (U133A and U133B) were ranked by the ABS (t-statistic). Genes were selected for further study based on a t-statistics cutoff of 2 or above 2. A negative t-statistic value indicates a decrease in, and positive indicates an increase in the expression of genes in cancer tissues. The fold-change in the expression of genes in Metastatic, Gleason grade 9 and Gleason grade 6 as compared to adjacent benign tissues are shown at the right.

Quantitative Real-Time Reverse Transcriptase-PCR. Seven down-regulated genes and four up-regulated genes were selected for validation by Taqman real-time RT-PCR to confirm the micorarray-based differential expression of these genes. One (1) μl of the cDNA was used in the PCR reactions. Taqman real-time primers and probes were obtained from Applied Biosystems (Foster City, Calif.) for all genes, except that the primers and probe for FABP5 were designed by the present inventors and custom synthesized. The sequence of the forward and reverse primers used for FABP5 were as follows:

(SEQ ID NO:27)

| | |
|---|---|
| forward primer: | GGAGTGGGATGGGAAGGAAAG; |

(SEQ ID NO:28)

| | |
|---|---|
| reverse primer: | CACTCCACCACTAATTTCCCATCTT; |
| reporter 1 Dye: | FAM; |
| reporter 1 quencher: | NFQ. |

All probes were labeled at the 5′ end with the reporter dye 6-carboxyfluorescein (6′-FAM) and at 3′ end with a nonfluorescent quencher NFQ. All PCR reactions were carried out in TaqMan® Universal PCR master mix (PE Applied Biosystems) with 900 nM of each primer and 250 nM of probe in a final volume of 50 μl. Thermal cycling conditions were as follows: 2 min at 50° C., with denaturation at 95° C. for 10 min, 40 cycles of 15 s at 95° C. (melting) and 1 min at 60° C. (annealing and elongation). The reactions were performed in an ABI Prism® 7700 Sequence Detection System.(PE Applied Biosystems). Standard curves were generated for the housekeeping gene, glyceraldehyde-3-phosphate-dehydrogenase (Applied Biosystems, part number 402869) to enable normalization of each gene. Data were expressed as relative fold changes in the mRNA expression by benign tissues after normalization with GAPDH levels (see FIG. 1 and TABLE 4).

TABLE 4
Text corresponding to FIG. 1.

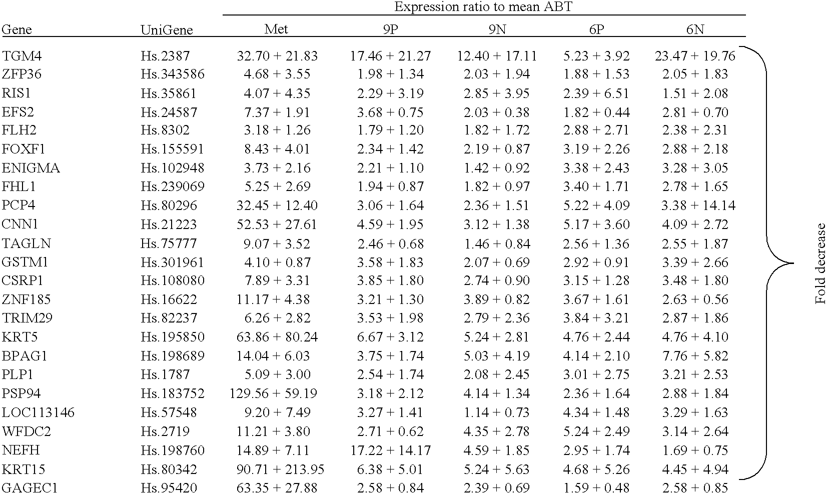

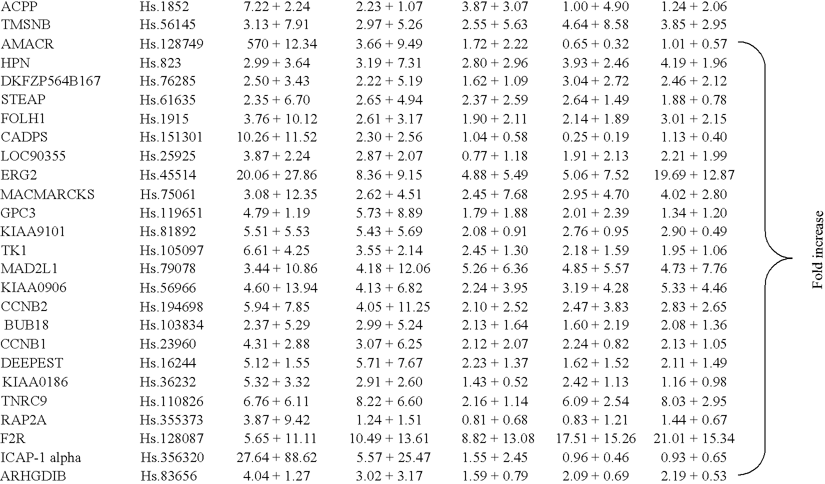

Cell Lines and 5-Aza-CdR Treatment. The human prostate cancer cell lines LNCaP, PC3 (American Type Culture Collection, Rockville, Md., USA) and LAPC4 (a gift from Dr. Charles L. Sawyers, University of California, Los Angeles, Calif.) were grown in Roswell Park Memorial Institute (RPMI) 1640 medium supplemented with 5% fetal bovine serum (FBS) at 37° C. and 5% CO₂ until reaching approximately 50-70% confluence. Cells were then treated with 5% FBS RPMT 1640 containing 6 μM 5-Aza-CdR (Sigma Chemicals Co., St. Louis, Mo.) for 6 days, with medium changes on day 1, 3, and 5. Total RNA was isolated from the cell lines and the expression of the genes was analyzed by TaqMan® real-time PCR as described above. Data were expressed as relative fold change in the mRNA expression by untreated controls (see FIG. 2).

Results:

In the study of EXAMPLE I herein, fifty (50) genes were identified and disclosed that are significantly altered in prostate cancer tissues. In this EXAMPLE, we used oligonucleotide s U133A and U133B chips containing 44792 transcripts. After hybridization of mRNA to the oliginucleotide s raw data was collected and the hybridization intensity for each gene expression is computed as the average of the probes within each probe set of a gene in the chips. Six hundred twenty-four (624) genes were ‘extracted’ from the metastatic vs. benign tissues with significant p-value <0.01 for differential expression (see TABLE 2 herein below).

The genes from the combined set of probes (U133A and U133B) are ordered by the ABS (t-statistic). For further validation, genes with t-statistics cutoff of 2 or above 2 were selected.

624 genes are disclosed that are significantly altered in cancer tissues. In particular cases, the results are consistent with previous findings of the upregulation and down regulation of particular genes in prostate cancer (Dhanasekaran et al., Nature 412:822-826, 2001; Luo et al., Cancer Res. 61:4683-4688, 2001; Magee et al., Cancer Res. 61:5692-5696, 2001; Welsh et al., Cancer Res. 61:5974-5978, 2001; Rubin et al., J. Amer. Med. Assn. 287:1662-1670, 2002; Ernst et al., Am. J. Pathol. 160:2169-2180, 2002; Sakai et al., Prostate 38:278-284, 1999).

According to the present invention, the alteration in the expression profiles of the genes is highly associated with prostate cancer progression and potentially can be useful biomarkers for predicting progression of the cancer.

The validated genes include seven (7) down-regulated genes, and four (4) up-regulated genes. Specifically, the validated down-regulated genes include: Supervillin (SVIL); Proline rich membrane anchor 1 (PRIMA1); TU3A; FLJ14084; KIAA1210; Sorbin and SH3 domain containing 1 (SORBS1); and C21orf63. The validated up-regulated genes include: MARCKS-like protein (MLP); SRY (sex determining region Y)-box 4 (SOX4); Fatty acid binding protein 5 (FABP5); and MAL2.

Validation confirmed the -based strong inverse correlation in the expression of all seven down-regulated genes (SVIL, PRIMA1, TU3A, FLJ14084; KIAA1210, SORBS1 and C21orf63) with progression of prostate cancer.

Likewise, validation confirmed the microarray-based correlation of increased expression, in Gleason grade 6 and Gleason grade 9 tissues, for all four upregulated genes (MLP, SOX4, FABP5 and MAL2).

Furthermore, the mRNA expression levels of the FLJ14084, SVIL, KIAA1210, PRIMA1 and TU3A genes in prostate cancer cell lines were restored by treatment of cells with 5-aza-2′-deoxycytidine, an inhibitor of DNA methylation, thereby implicating the transcriptional silencing of these genes by methylation in prostate cancer cells, and indicating that genomic DNA methylation is correlated with prostate tumorigenesis.

According to aspects of the present invention, the altered methylation and/or expression of these genes provide for novel diagnostic and/or prognostic assays for detection of precancerous and cancerous lesions of the prostate. The inventive compositions and methods have great utility as independent and/or supplementary approaches to standard histopathological work-up of precancerous and cancerous lesions of the prostate.

SVIL, a 205-kDa actin-binding protein is characterized as coregulator of the androgen receptor. Supervillian has shown to enhance the androgen receptor transactivation in muscle and other cells.

PRIMA1 is a membrane anchor of acetylcholinesterase. As a tetramer, acetylcholinesterase is anchored to the basal lamina of the neuromuscular junction and to the membrane of neuronal synapses. PRIMA anchors acetylcholinesterase in brain and muscle cell membranes.

TU3A gene is located in a commonly deleted region on 3p14.3-p14.2 in renal cell carcinoma. This gene encodes a protein consisting of 144 amino acids.

FLJ14084 and KIAA1210 genes maps on chromosome X at positions Xq22.1 and Xq24. The functions of these genes are unknown.

SORBS1 is an actin binding cytoskeletal protein involved in cell-matrix adhesion.

C21orf63 (human chromosome 21 open reading frame 63) encodes a protein with two D-galactoside/L-rhamnose binding SUEL domains.

MLP a macrophage myristolylated alanine rich C kinase substrate related protein encodes a MARCKS-like protein, a substrate for PKC.

SOX4 is a HMG (high mobility group) box 4 transcription factor involved in the regulation of embryonic development and in the determination of cell fate.

FABP5 (psoriasis associated) belongs to a family of small, highly conserved, cytoplasmic proteins that bind long-chain fatty acids and other hydrophobic ligands. FABPs roles include fatty acid uptake, transport and metabolism.

MAL2, an integral membrane protein of the MAL family, is an essential component of the machinery necessary for the indirect transcytotic route of apical transport in hepatoma HepG2 cells. The gene MAL2 is localized to chromosomal band 8q23 and potentially implicates TPD52-like proteins in vesicle transport.

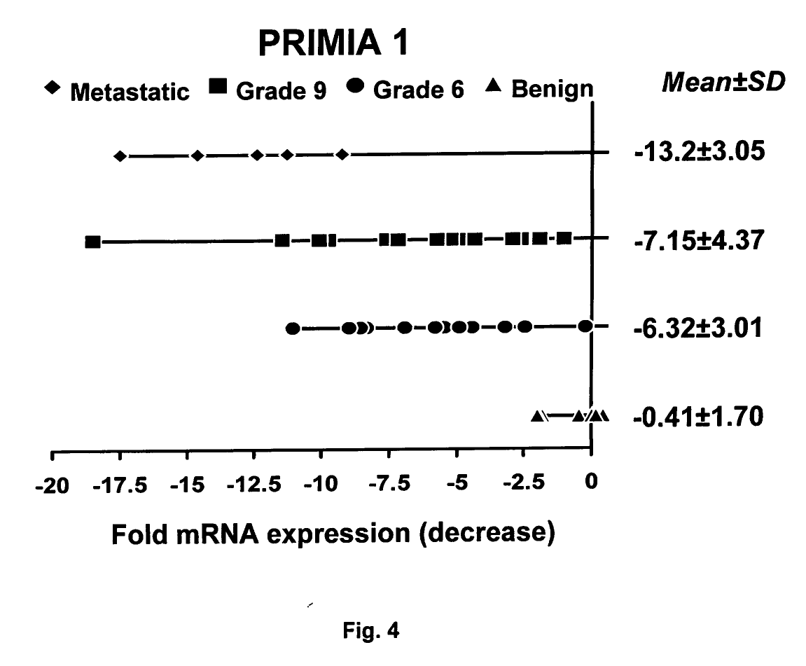

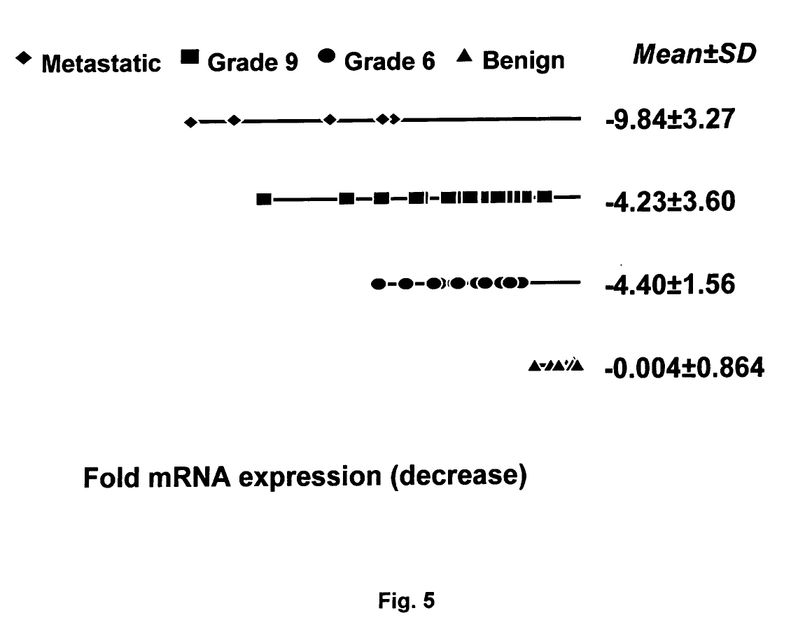

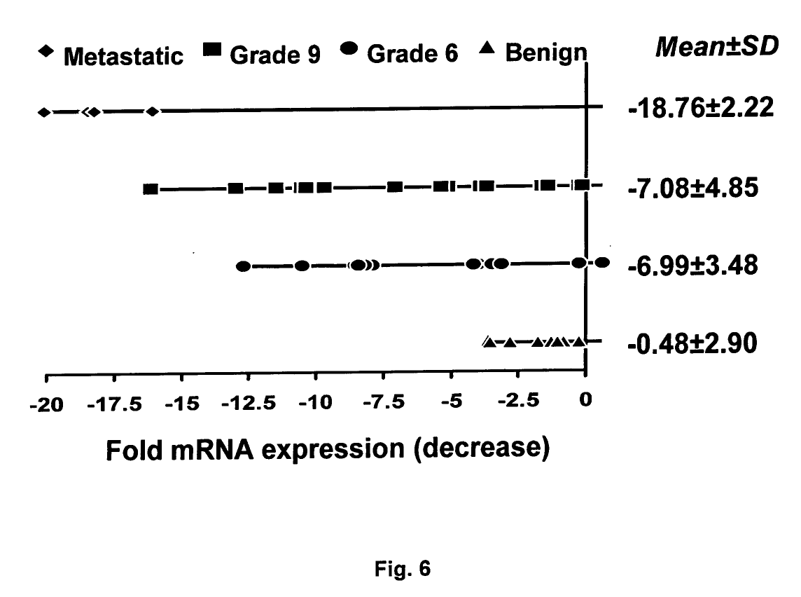

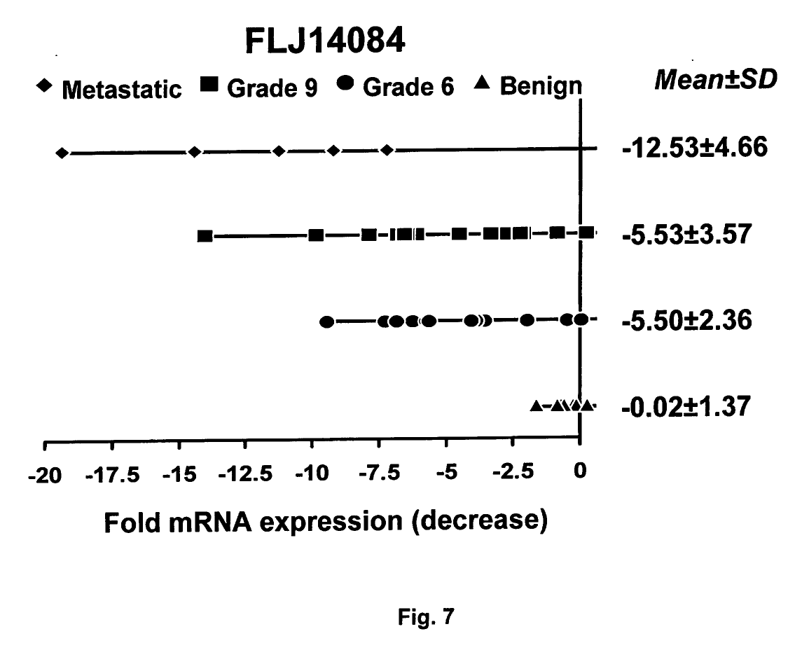

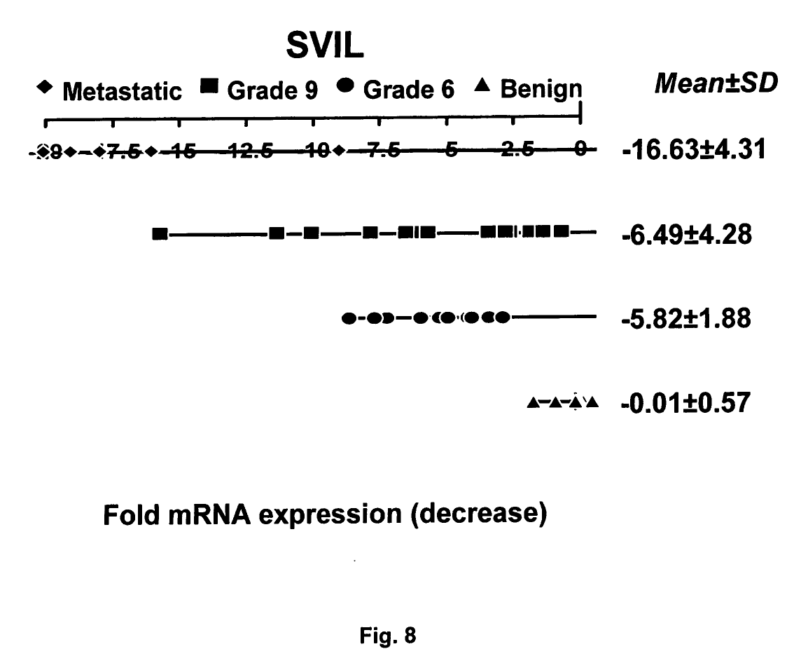

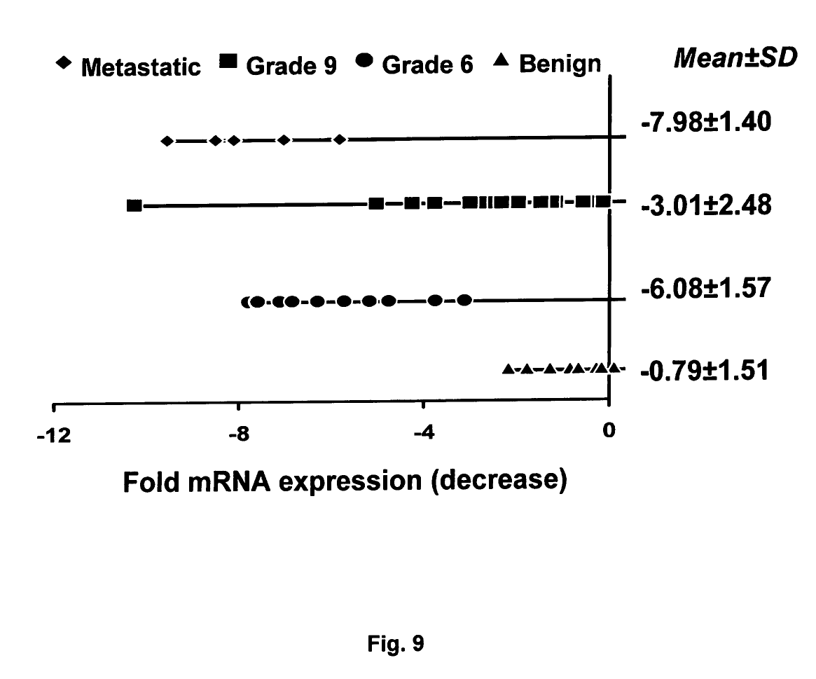

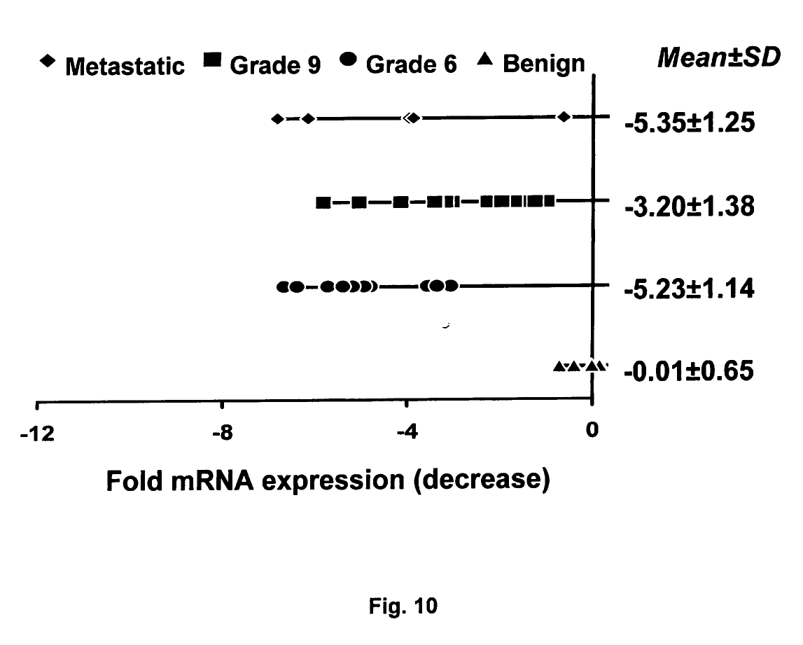

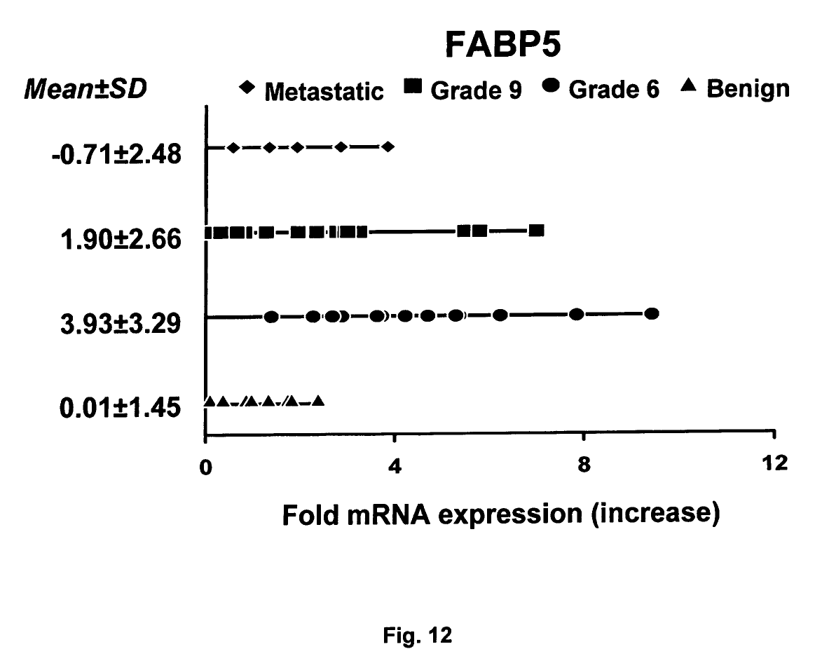

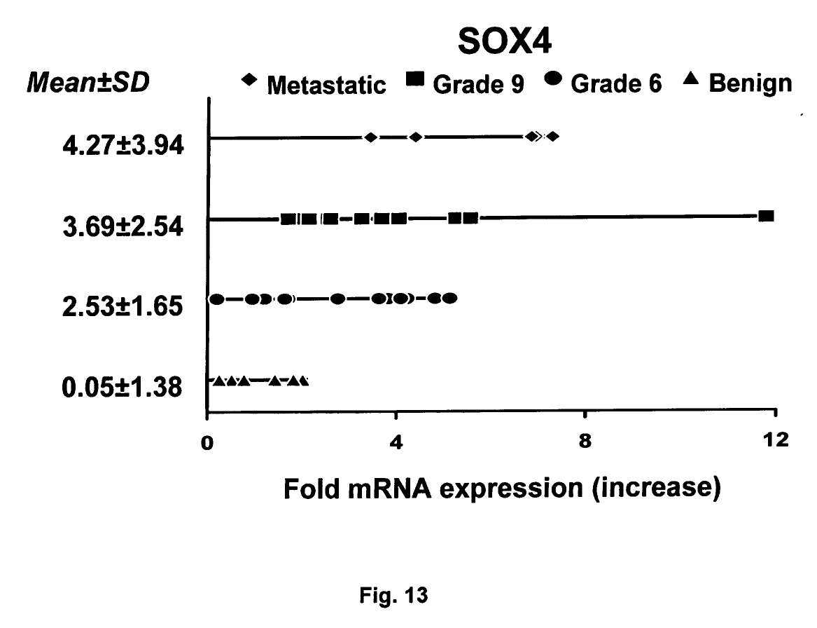

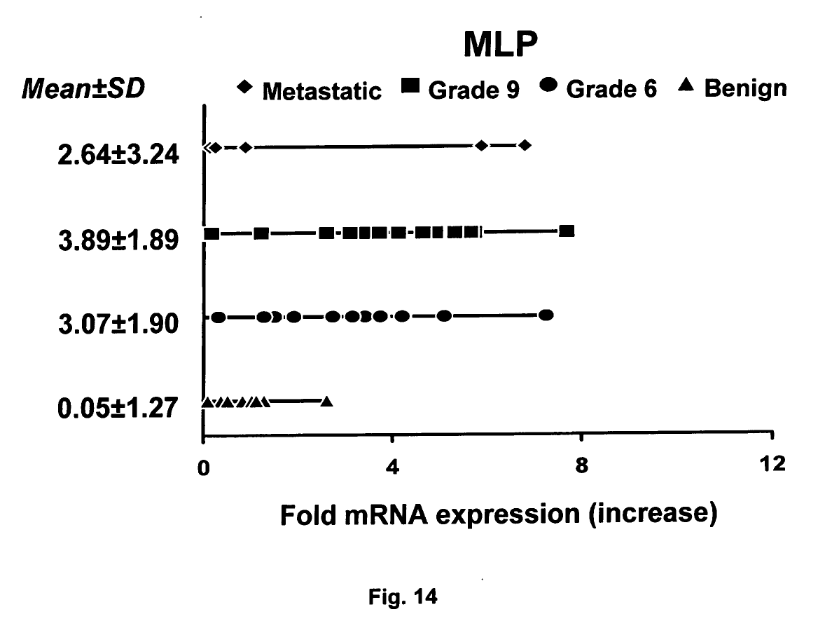

Specifically, eleven (11) genes were validated by real time PCR to confirm the. The Kruskal-Wallis global test was done with the real-time quantitative analysis for all the genes (FIGS. 4-14).

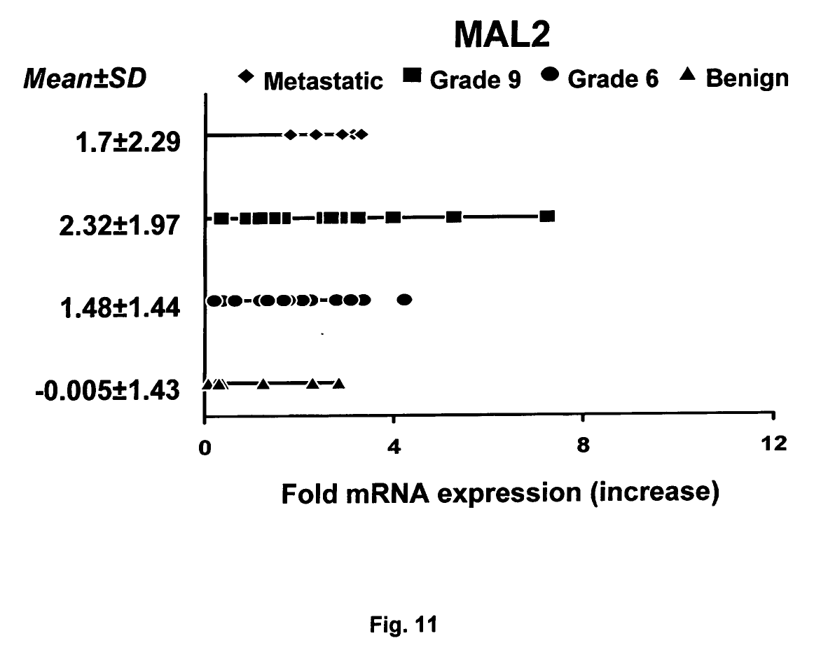

FIGS. 4-14 show, respectively, the expression levels of eleven genes (PRIMA1, TU3A, KIAA1210, FLJ14084; SVIL, SORBS1, C21orf63, MAL2, FABP5, SOX4 and MLP) as validated by Taqman real-time PCR analysis (including the Kruskal-Wallis global test) in 40 prostate tissue samples and expressed as the relative fold increase (MAL2, FABP5, SOX4 and MLP; FIGS. 11-14, respectively) or decrease (PRIMA1, TU3A, KIAA1210, FLJ14084; SVIL, SORBS1 and C21orf63; FIGS. 4-10, respectively) in the mRNA expression over the adjacent benign tissues after normalization to the house-keeping gene GAPDH mRNA levels. Mean and standard deviations are shown on the right. This real-time PCR data validates results from the instant-based expression analysis.

Therefore, as shown in FIGS. 4-10 and Table 3, a significant decrease in the expression of the PRIMA1, TU3A, KIAA1210, FLJ14084; SVIL, SORBS1 and C21orf63 genes was confirmed in metastatic versus organ confined and localized tumors compared to benign tissues (p<0.0004), and the decrease in the expression in prostate tumors indicates that they may play an important role in the development and progression of prostate cancer.

Validation of the MAL2, FABP5, SOX4 and MLP genes revealed a significant upregulation in the expression in Gleason grade 6 and Gleason grade 9 tissues compared to the metastatic tissues (FIGURES 11-14 and Table 3). The increase in mRNA levels of MAL2, MLP, SOX4 and FABP5 in cancer tissues indicates a role in prostate cancer development.

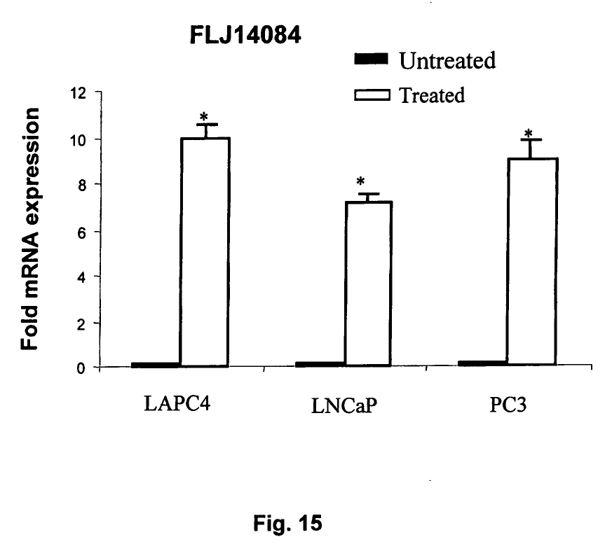

Transcriptional silencing. Additionally, to study the possibility of transcriptional silencing of the above-described down-regulated genes in prostate cancer, prostate cancer cells (LAPC4, LNCaP and PC3 cell lines) were treated with an inhibitor of DNA methylation, 5-aza-2-deoxycytidine(5-Aza-CdR) (see Vanaja et al 2003, supra, for methodology) (see FIGS. 15-19, for analysis the FLJ14084, SVIL, KIAA1210, PRIMA1 and TU3A genes, respectively)

FIG. 15 shows that a significant increase in the expression of FLJ14084 mRNA levels was found in all three prostate cancer cells tested.

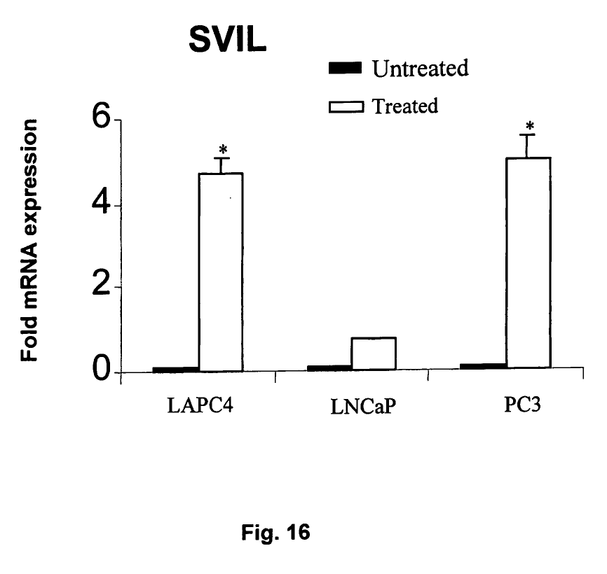

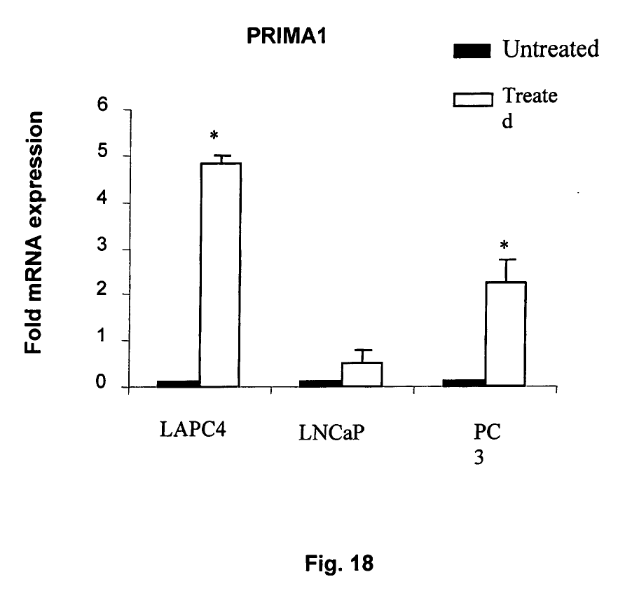

FIGS. 16 and 18, respectively, show that Supervillin (SVIL) and PRIMA1 exhibited a significant increase in LAPC4 and PC3 cells but not in LACaP.

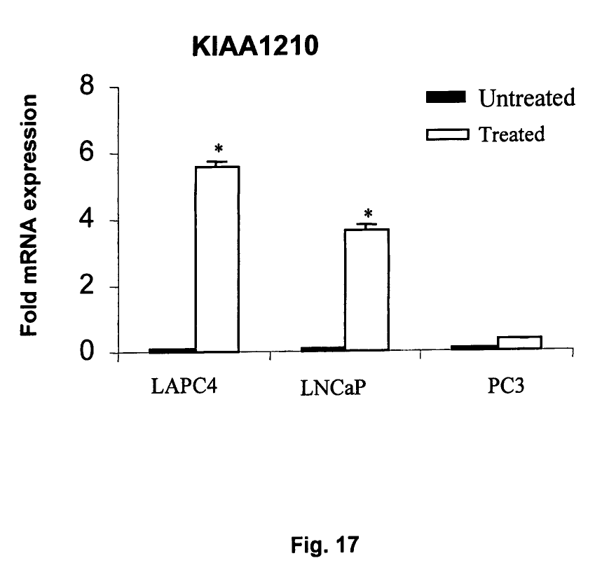

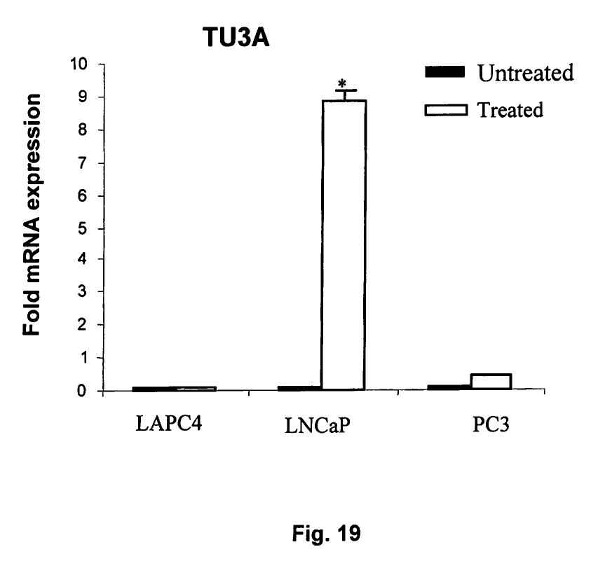

FIGS. 17 and 19, respectively, show that KIAA1210 mRNA levels were increased in LAPC4 and LNCaP cells, and that TU3A expression levels were significantly increased in LNCaP cells but not in LAPC4 and PC3 cells.

The increase in the mRNA levels of FLJ14084, SVIL, PRIMA1, KIAA1210 and TU3A by 5-Aza-CdR indicates that the gene is silenced by methylation in prostate cancer cells.

Therefore, mRNA expression profiling with oligonucleotide s identified 624 genes, the differential expression of which distinguishes and characterizes prostate cancer and benign prostatic tissues.

A decrease in the expression of seven downregulated genes was confirmed by real-time PCR analysis and validates a statistically significant correlation with prostate cancer progression. Restoration of the mRNA expression of FLJ14084, SVIL, KIAA1210, PRIMA1 and TU3A by a DNA methylation inhibitor indicates that the genes are, at least in part, silenced by DNA methyl at ion.

Increase of SOX4, MLP, FABP5 and MAL2 levels indicates a role in development and/or progression of prostate cancer.

Significantly, this is the first study to identify alteration in the expression of these eleven genes in patients with advanced prostate cancer, and they may serve as an independent and/or adjunct marker of prostate cancer aggressiveness.

TABLE 1
Prostate tissue samples with preoperative PSA values at diagnosis, Gleason histological
scores, and metastasis status of the tissues. A total of 40 prostate tissues were
used to study the gene expression profiling.

| Grade | ID | Age | % of tumor | Preop PSA | TNM (97) | Ploidy | METS |
|---|---|---|---|---|---|---|---|
| Grade 6 | 1 | 55 | 90 | 9.4 | T2b, N0− | Diploid | |
| | 2 | 50 | 80 | 7.5 | T2b, N0− | Tetraploid | |
| | 3 | 57 | 80 | 10.3 | T2b, N0− | Diploid | |
| | 4 | 67 | 80 | 16.7 | T2b, N0− | Diploid | |
| | 5 | 68 | 90 | 8.1 | T2a, N0− | Diploid | |
| | 6 | 71 | 95 | 17.1 | T2b, N1+ | Aneuploid | |
| | 7 | 61 | 80 | 5.2 | T2b, N0+ | Diploid | |
| | 8 | 71 | 100 | 41 | T2b, N0+ | Diploid | |
| | 9 | 65 | 75 | 7 | T2a, N0+ | Diploid | |
| | 10 | 51 | 70 | 14.3 | T2b, N0+ | Diploid | |
| | 11 | 66 | 90 | 23.5 | T2b, N0+ | Tetraploid | |
| | 12 | 65 | 80 | 6.5 | T2b, NO− | Diploid | |
| Grade 9 | 1 | 67 | 90 | 21.6 | T3aN0 | Tetraploid | |
| | 2 | 65 | 80 | 29.4 | T3bN0 | Tetraploid | |
| | 3 | 65 | 75 | 24.9 | T3bN0 | Tetraploid | |
| | 4 | 54 | 80 | 50 | T3bN0 | Tetraploid | |
| | 5 | 59 | 75 | 25.8 | T3bN0 | Diploid | |
| | 6 | 61 | 90 | 3.5 | T3aN0 | Aneuploid | |
| | 7 | 72 | 90 | 2.5 | T3bN0 | Tetraploid | |
| | 8 | 57 | 90 | 0.22 | T3aN0 | Aneuploid | |
| | 9 | 71 | 70 | 8.9 | T3aN0 | Diploid | |
| | 10 | 66 | 100 | 4.5 | T3a, N0+ | Diploid | |
| | 11 | 65 | 75 | 6.69 | T3b, N0+ | Tetraploid | |
| | 12 | 76 | 100 | 7.6 | T3b, N1+ | Diploid | |
| | 13 | 71 | 100 | 467 | T3b, N0+ | Aneuploid | |
| | 14 | 69 | 70 | 5.6 | T3b, No+ | Diploid | liver, bone |
| | 15 | 66 | 100 | 2.9 | T3b, N1− | Aneuploid | |

| | | | | | |
|---|---|---|---|---|---|
| Metastatic | M 1 | 62 | 90 | | Metastatic lesion to liver |
| | M 2 | | | | Peritoneal implant |
| | M 3 | | | | Lymph |
| | | | | | node |
| | M 4 | | | | Lymph |
| | | | | | node |
| | M 5 | 68 | 90 | 8.9 | Metastatic prostate cancer in lung. |

TABLE 2
Differential expression (relative to benign tissue) of 624 significantly regulated genes in 40 prostate
tissue samples. The expression is computed as the average of the probes within each
probe set of a gene in the chips. The 624 genes were ‘extracted’ from the
metastatic vs. benign tissues with significant p-value <0.01. The genes from the
combined set of probes (U133A and U133B) were ranked by the ABS (t-statistic).
Genes were selected for further study based on a t-statistics cutoff of 2 or above 2.
A negative t-statistic value indicates a decrease in, and positive indicates an increase
in the expression of genes in cancer tissues. The fold-change in the expression of
genes in Metastatic, Gleason grade 9 and Gleason grade 6 as compared
to adjacent benign tissues are shown at the right.

| Affymetrix | | | | Metastatic | | Fold Change | | |
|---|---|---|---|---|---|---|---|---|
| ProbeSetName | Genbank | Unigene | Metastatic p-value | t-statistic | Gene | Met-Nrml | Gs-Nrml | Gs-Nrml |
| 202274_at | NM_001615.2 | Hs.378774 | 0 | −22.5051 | ACTG2 | 0.053803311 | 0.275524014 | 0.321307046 |
| 201496_x_at | AI889739 | Hs.78344 | 0 | −16.3756 | MYH11 | 0.092513093 | 0.311334938 | 0.392683897 |
| 200621_at | NM_004078.1 | Hs.108080 | 0 | −15.4063 | CSRP1 | 0.196300809 | 0.391723864 | 0.405003189 |
| 214027_x_at | AA889653 | Hs.279604 | 0 | −15.1949 | DES | 0.220582131 | 0.453197127 | 0.437336656 |
| 202555_s_at | NM_005965.1 | Hs.211582 | 0 | −14.5834 | MYLK | 0.106681549 | 0.320630291 | 0.341562201 |
| 205564_at | NM_007003.1 | Hs.95420 | 0 | −14.42 | GAGEC1 | 0.261255045 | 0.508938954 | 0.677749388 |
| 203951_at | NM_001299.1 | Hs.21223 | 0 | −14.2117 | CNN1 | 0.112656911 | 0.363696874 | 0.354889317 |
| 212730_at | AK026420.1 | Hs.10587 | 0 | −13.1138 | DMN | 0.140553471 | 0.332814198 | 0.356094906 |
| 207876_s_at | NM_001458.1 | Hs.58414 | 0 | −12.8903 | FLNC | 0.474950906 | 0.597498448 | 0.621066165 |
| 204083_s_at | NM_003289.1 | Hs.300772 | 0 | −12.1739 | TPM2 | 0.149184376 | 0.39284232 | 0.405764156 |
| 201058_s_at | NM_006097.1 | Hs.9615 | 0 | −12.1029 | MYL9 | 0.11968876 | 0.321698372 | 0.332586079 |
| 205547_s_at | NM_003186.2 | Hs.433399 | 0 | −12.0177 | TAGLN | 0.106828219 | 0.406442173 | 0.349395924 |
| 200974_at | NM_001613.1 | Hs.195851 | 0 | −11.5691 | ACTA2 | 0.17792117 | 0.463927526 | 0.40713061 |
| 209948_at | U61536.1 | Hs.93841 | 0 | −11.5427 | KCNMB1 | 0.362212251 | 0.556744547 | 0.560864417 |
| 201820_at | NM_000424.1 | Hs.433845 | 0 | −11.3437 | KRT5 | 0.280032698 | 0.384279156 | 0.429128229 |
| 226303_at | AA706788 | Hs.46531 | 0 | −10.9808 | PGM5 | 0.234867491 | 0.444812189 | 0.531081579 |
| 203766_s_at | NM_012134.1 | Hs.79386 | 0 | −10.5978 | LMOD1 | 0.258393922 | 0.503828085 | 0.466892497 |
| 205549_at | NM_006198.1 | Hs.80296 | 0 | −10.3913 | PCP4 | 0.135604995 | 0.384014747 | 0.345619693 |
| 226523_at | AI082237 | Hs.32978 | 0 | −10.3433 | PCSK7 | 0.540871217 | 0.722179949 | 0.625803398 |
| 211737_x_at | BC005916.1 | Hs.44 | 0 | −10.1922 | PTN | 0.372578608 | 0.706509794 | 0.925406566 |
| 221667_s_at | AF133207.1 | Hs.111676 | 0 | −10.0549 | H11 | 0.28591921 | 0.432577624 | 0.498592093 |
| 202504_at | NM_012101.1 | Hs.82237 | 0 | −9.8229 | TRIM29 | 0.362228754 | 0.451921947 | 0.466335609 |
| 211276_at | AF063606.1 | Hs.356068 | 0 | −9.7461 | MY048 | 0.518494652 | 0.718165729 | 0.697505604 |
| 205856_at | NM_015865.1 | Hs.171731 | 0 | −9.4026 | SLC14A1 | 0.423229445 | 0.555799182 | 0.581379854 |
| 213371_at | AI803302 | Hs.49998 | 0 | −9.1891 | LDB3 | 0.577603464 | 0.705513913 | 0.745367895 |
| 219478_at | NM_021197.1 | Hs.36688 | 0 | −8.9672 | WFDC1 | 0.306657563 | 0.57816262 | 0.539783258 |
| 202566_s_at | AF051851.1 | Hs.154567 | 0 | −8.9067 | SVIL | 0.56810571 | 0.664300973 | 0.616844465 |
| 225721_at | AI658662 | Hs.24192 | 0 | −8.7832 | SYNPO2 | 0.211455588 | 0.477462293 | 0.438029507 |
| 37005_at | D28124 | Hs.76307 | 0 | −8.7348 | NBL1 | 0.319533792 | 0.515936194 | 0.641274562 |
| 204400_at | NM_005864.1 | Hs.24587 | 0 | −8.7168 | EFS | 0.570344842 | 0.691853688 | 0.795672591 |
| 203370_s_at | NM_005451.2 | Hs.102948 | 0 | −8.606 | ENIGMA | 0.482541378 | 0.692765088 | 0.579424908 |
| 210297_s_at | U22178.1 | Hs.433392 | 0 | −8.564 | MSMB | 0.049869989 | 0.166938871 | 0.444403085 |
| 230595_at | BF677651 | — | 0 | −8.5487 | FLJ40899 | 0.387347112 | 0.507947468 | 0.570499488 |
| 210987_x_at | M19267.1 | Hs.77899 | 0 | −8.4458 | TPM1 | 0.287632225 | 0.446692011 | 0.445839571 |
| 213992_at | AI889941 | Hs.408 | 0 | −8.3452 | COL4A6 | 0.603412488 | 0.723897608 | 0.730134432 |
| 241350_at | AL533913 | Hs.86999 | 0 | −8.3425 | LOC283807 | 0.666081008 | 0.763231436 | 0.747271248 |
| 221246_x_at | NM_018274.2 | Hs.351432 | 0 | −8.3418 | TNS | 0.526103794 | 0.675841286 | 0.622485396 |
| 204734_at | NM_002275.1 | Hs.80342 | 0 | −8.3269 | KRT15 | 0.236632551 | 0.357945338 | 0.416315147 |
| 223623_at | AF325503.1 | Hs.43125 | 0 | −8.2904 | ECRG4 | 0.396258177 | 0.707056669 | 0.606054804 |
| 241879_at | AW511222 | Hs.296326 | 0 | −8.2151 | sp: P39189 | 0.582477482 | 1.020217149 | 0.915877876 |
| 205316_at | BF223679 | Hs.118747 | 0 | −8.1393 | SLC15A2 | 0.511602561 | 0.88612165 | 1.096600868 |
| 205132_at | NM_005159.2 | Hs.118127 | 0 | −8.1281 | ACTC | 0.445183351 | 0.562177326 | 0.635825598 |
| 218087_s_at | NM_015385.1 | Hs.108924 | 0 | −8.0964 | SORBS1 | 0.196441183 | 0.476915472 | 0.483022062 |
| 203296_s_at | NM_000702.1 | Hs.34114 | 0 | −8.0632 | ATP1A2 | 0.546867898 | 0.673105614 | 0.711571158 |
| 219090_at | NM_020689.2 | Hs.12321 | 0 | −7.877 | SLC24A3 | 0.630015865 | 0.827470089 | 0.756875262 |
| 209167_at | AF016004.1 | Hs.5422 | 0 | −7.8638 | GPM6B | 0.506791341 | 0.708935715 | 0.729964766 |
| 202822_at | AL044018 | Hs.180398 | 0 | −7.7949 | LPP | 0.414861492 | 0.665931121 | 0.621661858 |
| 227826_s_at | AW138143 | Hs.156880 | 0 | −7.7459 | IMAGE: 4791597 | 0.202170331 | 0.483537908 | 0.449814255 |
| 209863_s_at | AF091627.1 | Hs.137569 | 0 | −7.7045 | TP73L | 0.480129801 | 0.577410686 | 0.582774883 |
| 214752_x_at | AI625550 | Hs.195464 | 0 | −7.6432 | FLNA | 0.256719948 | 0.450881595 | 0.37282063 |
| 201957_at | AF324888.1 | Hs.130760 | 0 | −7.4586 | PPP1R12B | 0.350435619 | 0.590001393 | 0.477521857 |
| 209270_at | L25541.1 | Hs.75517 | 0 | −7.4324 | LAMB3 | 0.658071625 | 0.709333463 | 0.717732863 |
| 235468_at | AA531287 | Hs.11924 | 0 | −7.4106 | LOC339162 | 0.659275233 | 0.731812864 | 0.789170866 |
| 207390_s_at | NM_006932.1 | Hs.149098 | 0 | −7.4075 | SMTN | 0.283040393 | 0.441159739 | 0.389854498 |
| 207016_s_at | AB015228.1 | Hs.95197 | 0 | −7.3893 | ALDH1A2 | 0.450127957 | 0.616891031 | 0.631455824 |
| 228232_s_at | NM_014312.1 | Hs.112377 | 0 | −7.3768 | CTXL | 0.617402852 | 0.751970331 | 0.822702013 |
| 201431_s_at | NM_001387.1 | Hs.74566 | 0 | −7.376 | DPYSL3 | 0.44502532 | 0.658801891 | 0.583119459 |
| 214175_x_at | BE043700 | Hs.424312 | 0 | −7.3391 | RIL | 0.653610738 | 0.744219621 | 0.758834964 |
| 204491_at | R40917 | Hs.172081 | 0 | −7.3239 | PDE4D | 0.657929279 | 0.771456315 | 0.760289946 |
| 205265_s_at | NM_005876.1 | Hs.21639 | 0 | −7.3185 | APEG1 | 0.650580959 | 0.826154763 | 0.735291274 |
| 227827_at | AW138143 | Hs.156880 | 0 | −7.2467 | IMAGE: 4791597 | 0.205405593 | 0.486158058 | 0.444403587 |
| 219167_at | NM_016563.1 | Hs.27018 | 0 | −7.218 | RIS | 0.551508072 | 0.70270956 | 0.677791849 |
| 221584_s_at | U11058.2 | Hs.89463 | 0 | −7.1988 | KCNMA1 | 0.465638173 | 0.713011709 | 0.740351333 |
| 204990_s_at | NM_000213.1 | Hs.85266 | 0 | −7.1772 | ITGB4 | 0.640435624 | 0.673685098 | 0.651352082 |
| 200906_s_at | AK025843.1 | Hs.194431 | 0 | −7.0866 | KIAA0992 | 0.559112821 | 0.708081908 | 0.639547875 |
| 227727_at | H15920 | Hs.118513 | 0 | −7.0704 | MGC21621 | 0.503312422 | 0.723243606 | 0.684342661 |
| 213675_at | W61005 | Hs.424272 | 0 | −6.9873 | FLJ46049 fis | 0.648174796 | 0.82023855 | 0.773977519 |
| 216264_s_at | X79683.1 | Hs.90291 | 0 | −6.9284 | LAMB2 | 0.612076466 | 0.754958113 | 0.76493073 |
| 204931_at | NM_003206.1 | Hs.78061 | 0 | −6.8922 | TCF21 | 0.505430709 | 0.809029779 | 0.826637353 |
| 203585_at | NM_007150.1 | Hs.16622 | 0 | −6.8917 | ZNF185 | 0.505830837 | 0.615699181 | 0.615001687 |
| 214505_s_at | AF220153.1 | Hs.239069 | 0 | −6.8661 | FHL1 | 0.354969836 | 0.565246533 | 0.478041452 |
| 225524_at | AU152178 | Hs.5897 | 0 | −6.8558 | ANTXR2 | 0.409339229 | 0.677654832 | 0.830447277 |
| 208789_at | BC004295.1 | Hs.29759 | 0 | −6.7973 | PTRF | 0.48382159 | 0.606341207 | 0.598833579 |
| 229578_at | AA716165 | Hs.134933 | 0 | −6.7872 | JPH2 | 0.611911671 | 0.753071229 | 0.719712403 |
| 204069_at | NM_002398.1 | Hs.170177 | 0 | −6.7853 | MEIS1 | 0.477877704 | 0.742008585 | 0.615699332 |
| 204268_at | NM_005978.2 | Hs.38991 | 0 | −6.6896 | S100A2 | 0.644792961 | 0.724799993 | 0.709511387 |
| 203687_at | NM_002996.1 | Hs.80420 | 0 | −6.6537 | CX3CL1 | 0.604335928 | 0.70778563 | 0.696839146 |
| 226047_at | N66571 | Hs.432673 | 0 | −6.6187 | MRVI1 | 0.54659298 | 0.764619642 | 0.704681576 |
| 229339_at | AI093327 | Hs.42128 | 0 | −6.6142 | MYOCD | 0.652300902 | 0.762761259 | 0.742382465 |
| 204455_at | NM_001723.1 | Hs.198689 | 0 | −6.6119 | BPAG1 | 0.437282846 | 0.553091326 | 0.529050223 |
| 227188_at | AI744591 | Hs.30156 | 0 | −6.5874 | C21ORF63 | 0.627711098 | 0.742259445 | 0.734336678 |
| 212236_x_at | Z19574 | Hs.2785 | 0 | −6.5682 | KRT17 | 0.244018067 | 0.354016876 | 0.391642401 |
| 211864_s_at | AF207990.1 | Hs.234680 | 0 | −6.5289 | FER1L3 | 0.638621974 | 0.717399972 | 0.721878751 |
| 221541_at | AL136861.1 | Hs.262958 | 0 | −6.4859 | DKFZP434B044 | 0.41721507 | 0.599924344 | 0.641831035 |
| 227688_at | AK022128.1 | Hs.65366 | 0 | −6.4684 | KIAA1495 | 0.633294812 | 0.814358954 | 0.815206337 |
| 219685_at | NM_021637.1 | Hs.45140 | 0 | −6.4435 | FLJ14084 | 0.586063163 | 0.717268449 | 0.72677563 |
| 212148_at | BF967998 | Hs.21851 | 0 | −6.4376 | PBX1 | 0.42188315 | 0.739252199 | 0.739111604 |
| 203892_at | NM_006103.1 | Hs.2719 | 0 | −6.4309 | WFDC2 | 0.442888969 | 0.528585158 | 0.527606737 |
| 206938_at | NM_000348.1 | Hs.1989 | 0.0001 | −6.2511 | SRD5A2 | 0.645321331 | 0.709715832 | 0.700927697 |
| 203453_at | NM_001038.1 | Hs.2794 | 0.0001 | −6.2336 | SCNN1A | 0.398698168 | 0.714327568 | 0.59825747 |
| 208131_s_at | NM_000961.1 | Hs.302085 | 0.0001 | −6.2334 | PTGIS | 0.55428096 | 0.707921871 | 0.663877631 |
| 225328_at | BF693502 | Hs.6630 | 0.0001 | −6.2159 | FBXO32 | 0.554087468 | 0.725502261 | 0.670659094 |
| 229947_at | AI088609 | Hs.98558 | 0.0001 | −6.215 | FLJ26876 fis | 0.339316921 | 0.587017326 | 1.271328015 |
| 209283_at | AF007162.1 | Hs.391270 | 0.0001 | −6.2045 | CRYAB | 0.48330264 | 0.605081516 | 0.606280623 |
| 238877_at | BE674583 | Hs.102408 | 0.0001 | −6.1438 | EYA4 | 0.657537486 | 0.800115833 | 0.76159609 |
| 212647_at | NM_006270.1 | Hs.9651 | 0.0001 | −6.0582 | RRAS | 0.654375113 | 0.704479436 | 0.746177433 |
| 201787_at | NM_001996.1 | Hs.79732 | 0.0001 | −5.9802 | FBLN1 | 0.464771633 | 0.665149327 | 0.666501329 |
| 202054_s_at | NM_000382.1 | Hs.159608 | 0.0001 | −5.9675 | ALDH3A2 | 0.596718306 | 0.72605588 | 0.839818723 |
| 201022_s_at | NM_006870.2 | Hs.82306 | 0.0001 | −5.9596 | DSTN | 0.469263509 | 0.735850647 | 0.812634097 |
| 204418_x_at | NM_000848.1 | Hs.279837 | 0.0001 | −5.9382 | GSTM2 | 0.48069341 | 0.583085624 | 0.513812759 |
| 203571_s_at | NM_006829.1 | Hs.74120 | 0.0001 | −5.9171 | APM2 | 0.341804932 | 0.546438229 | 0.568429103 |
| 218418_s_at | NM_015493.1 | Hs.284208 | 0.0001 | −5.9077 | KIAA1518 | 0.584255705 | 0.705547521 | 0.626408504 |
| 221004_s_at | NM_030926.1 | Hs.111577 | 0.0001 | −5.8947 | ITM2C | 0.653257154 | 0.736561823 | 0.83311969 |
| 209651_at | BC001830.1 | Hs.25511 | 0.0001 | −5.8551 | TGFB1I1 | 0.458573659 | 0.578853882 | 0.600982832 |
| 242447_at | AI656180 | Hs.359230 | 0.0001 | −5.7774 | IMAGE2243078 | 0.558245981 | 0.699712197 | 0.721118844 |
| 225990_at | BF343163 | Hs.339352 | 0.0001 | −5.7608 | BOC | 0.554456141 | 0.856383743 | 0.767316078 |
| 200824_at | NM_000852.2 | Hs.226795 | 0.0001 | −5.7489 | GSTP1 | 0.62528976 | 0.713573555 | 0.619455086 |
| 220765_s_at | NM_017980.1 | Hs.127273 | 0.0001 | −5.7238 | LIMS2 | 0.583795105 | 0.720887886 | 0.650878707 |
| 218980_at | NM_025135.1 | Hs.288841 | 0.0001 | −5.6835 | KIAA1695 | 0.555775824 | 0.739032946 | 0.63430201 |
| 226755_at | AI375939 | Hs.301885 | 0.0001 | −5.652 | NPC-A-5 | 0.504552312 | 0.607434268 | 0.586917627 |
| 212992_at | AI935123 | Hs.57548 | 0.0002 | −5.6427 | C14ORF78 | 0.564503996 | 0.748557853 | 0.700982305 |
| 212233_at | AL523076 | Hs.82503 | 0.0002 | −5.6365 | MAP1B | 0.44160083 | 0.750965592 | 0.557666109 |
| 206104_at | NM_002202.1 | Hs.505 | 0.0002 | −5.6175 | ISL1 | 0.575277922 | 0.881067783 | 0.809109438 |
| 204163_at | NM_007046.1 | Hs.63348 | 0.0002 | −5.6011 | EMILIN1 | 0.634511395 | 0.758346646 | 0.684017738 |
| 227742_at | AI638295 | Hs.353146 | 0.0002 | −5.5979 | CLIC6 | 0.670703561 | 0.790469935 | 0.748444013 |
| 202949_s_at | NM_001450.1 | Hs.8302 | 0.0002 | −5.5713 | FHL2 | 0.415411095 | 0.601046867 | 0.508834921 |
| 225809_at | AI659927 | Hs.6634 | 0.0002 | −5.546 | DKFZP564O0823 | 0.395102331 | 0.525825047 | 0.676752728 |
| 228640_at | BE644809 | Hs.339315 | 0.0002 | −5.5441 | PCDH7 | 0.480531518 | 0.688388165 | 0.607218477 |
| 220595_at | NM_013377.1 | Hs.380044 | 0.0002 | −5.5383 | DKFZP434B0417 | 0.57489509 | 0.73680738 | 0.725634819 |
| 227850_x_at | AW084544 | Hs.352987 | 0.0002 | −5.4802 | CDC42EP5 | 0.477969665 | 0.596031808 | 0.968440186 |
| 226304_at | AA563621 | Hs.351558 | 0.0002 | −5.4353 | FLJ32389 | 0.530655476 | 0.6934539 | 0.754666976 |
| 209291_at | NM_001546.1 | Hs.34853 | 0.0002 | −5.4154 | ID4 | 0.455232047 | 0.721342896 | 0.566598287 |
| 215333_x_at | X08020.1 | Hs.301961 | 0.0002 | −5.3931 | GSTM1 | 0.592136213 | 0.684406135 | 0.62699488 |
| 216331_at | AK022548.1 | Hs.74369 | 0.0002 | −5.3927 | ITGA7 | 0.619618876 | 0.766675236 | 0.668484029 |
| 226103_at | AF114264.1 | Hs.216381 | 0.0002 | −5.3885 | NEXILIN | 0.525120912 | 0.768419067 | 0.703204986 |
| 235342_at | AI808090 | Hs.159425 | 0.0002 | −5.3861 | SPOCK3 | 0.484383621 | 0.779581929 | 0.754636038 |
| 207480_s_at | NM_020149.1 | Hs.104105 | 0.0002 | −5.3838 | MEIS2 | 0.400172683 | 0.620471855 | 0.648818113 |
| 214724_at | AF070621.1 | Hs.61408 | 0.0002 | −5.3704 | SECP43 | 0.581948345 | 0.79632702 | 0.894707932 |
| 204894_s_at | NM_003734.2 | Hs.198241 | 0.0002 | −5.3659 | AOC3 | 0.531891736 | 0.640777537 | 0.671825828 |
| 204570_at | NM_001864.1 | Hs.114346 | 0.0002 | −5.3611 | COX7A1 | 0.583822659 | 0.688692839 | 0.667070979 |
| 227386_s_at | N63821 | Hs.268024 | 0.0002 | −5.3428 | DKFZp434C184 | 0.627647025 | 0.8254192 | 0.735537074 |
| 203476_at | NM_006670.1 | Hs.82128 | 0.0002 | −5.3172 | TPBG | 0.539920131 | 0.832778932 | 0.744024144 |
| 204442_x_at | NM_003573.1 | Hs.85087 | 0.0002 | −5.3088 | LTBP4 | 0.600486893 | 0.851972293 | 0.793883461 |
| 225662_at | BE620734 | Hs.115175 | 0.0003 | −5.2651 | ZAK | 0.55234581 | 0.787517538 | 0.727394698 |
| 212135_s_at | AW517686 | Hs.343522 | 0.0003 | −5.2353 | ATP2B4 | 0.636641448 | 0.732189085 | 0.630131357 |
| 203256_at | NM_001793.1 | Hs.2877 | 0.0003 | −5.1976 | CDH3 | 0.647266558 | 0.766651139 | 0.779882388 |
| 212599_at | AK025298.1 | Hs.32168 | 0.0003 | −5.1555 | AUTS2 | 0.590495727 | 0.899171353 | 0.757428451 |
| 214880_x_at | D90453.1 | Hs.325474 | 0.0003 | −5.1539 | CALD1 | 0.652622749 | 0.773522151 | 0.728499496 |
| 223315_at | AF278532.1 | Hs.102541 | 0.0003 | −5.1344 | NTN4 | 0.609203042 | 0.694091861 | 0.676407558 |
| 237206_at | AI452798 | Hs.42128 | 0.0003 | −5.1273 | MYCD | 0.570277407 | 0.714769249 | 0.725829487 |
| 200930_s_at | AA156675 | Hs.75350 | 0.0003 | −5.1226 | VCL | 0.57672027 | 0.704478779 | 0.716474363 |
| 205935_at | NM_001451.1 | Hs.155591 | 0.0003 | −5.1024 | FOXF1 | 0.518061956 | 0.716512988 | 0.668534803 |
| 227006_at | AA156998 | Hs.348037 | 0.0004 | −5.0743 | PPP1R14A | 0.606215229 | 0.685190003 | 0.640681808 |
| 231096_at | AA226269 | Hs.104215 | 0.0004 | −5.0724 | GDEP | 0.466191103 | 0.819874985 | 1.698312318 |
| 228504_at | AI828648 | Hs.16757 | 0.0004 | −5.0489 | SCN7A | 0.660946973 | 0.894320027 | 0.869601383 |
| 211458_s_at | AF180519.1 | Hs.334497 | 0.0004 | −5.0473 | GABARAPL3 | 0.557236207 | 0.720987448 | 0.839166916 |
| 33767_at | X15306 | — | 0.0004 | −5.0434 | NEFH | 0.163714626 | 0.167695942 | 0.558788587 |
| 220617_s_at | NM_018181.1 | Hs.380730 | 0.0004 | −5.0414 | FLJ10697 | 0.464292261 | 0.673385903 | 0.715109709 |
| 225016_at | N48299 | Hs.374481 | 0.0004 | −5.0299 | APCDD1 | 0.507423231 | 0.73987269 | 0.764999022 |
| 209129_at | AF000974.1 | Hs.380230 | 0.0004 | −5.014 | TRIP6 | 0.642578679 | 0.734972834 | 0.69592588 |
| 227088_at | BF221547 | Hs.16578 | 0.0004 | −4.9968 | FLJ42757 | 0.440236546 | 0.753875498 | 0.690231264 |
| 214247_s_at | AU148057 | Hs.278503 | 0.0004 | −4.9761 | DKK3 | 0.448464785 | 0.637052822 | 0.617597889 |
| 219669_at | NM_020406.1 | Hs.232165 | 0.0004 | −4.9418 | PRV1 | 0.435784309 | 0.473668236 | 0.547428403 |
| 209074_s_at | AL050264.1 | Hs.8022 | 0.0005 | −4.9284 | TU3A | 0.474253246 | 0.571454355 | 0.643798262 |
| 204686_at | NM_005544.1 | Hs.96063 | 0.0005 | −4.9119 | IRS1 | 0.599920666 | 0.780445638 | 0.717289768 |
| 227194_at | BF106962 | Hs.20415 | 0.0005 | −4.8943 | FAM3B | 0.502784686 | 1.303068671 | 2.771161255 |
| 203373_at | NM_003877.1 | Hs.405946 | 0.0005 | −4.8781 | SOCS2 | 0.503022765 | 0.836972031 | 1.070200787 |
| 204940_at | NM_002667.1 | Hs.85050 | 0.0005 | −4.8415 | PLN | 0.631681514 | 0.815827405 | 0.771310785 |
| 206953_s_at | NM_012302.1 | Hs.24212 | 0.0005 | −4.8194 | LPHN2 | 0.654350027 | 0.827603625 | 0.776672002 |
| 204393_s_at | NM_001099.2 | Hs.1852 | 0.0006 | −4.8016 | ACPP | 0.115290032 | 0.329784847 | 0.855266897 |
| 205609_at | NM_001146.1 | Hs.2463 | 0.0006 | −4.7892 | ANGPT1 | 0.657951095 | 0.764380343 | 0.776848693 |
| 225782_at | BG171064 | Hs.339024 | 0.0006 | −4.7743 | LOC253827 | 0.458190603 | 0.67025752 | 0.614380899 |
| 213568_at | AI811298 | Hs.348363 | 0.0006 | −4.7513 | OSR2 | 0.595887145 | 0.817690588 | 0.802144853 |
| 201462_at | NM_014766.1 | Hs.75137 | 0.0006 | −4.7481 | KIAA0193 | 0.620924878 | 0.797802174 | 0.734057849 |
| 222043_at | AI982754 | Hs.75106 | 0.0006 | −4.7308 | CLU | 0.593038992 | 0.681315769 | 0.679106494 |
| 230087_at | AI823645 | Hs.356130 | 0.0006 | −4.7300 | PRIMA1 | 0.744276908 | 0.774136798 | 0.814308813 |
| 209763_at | AL049176 | Hs.82223 | 0.0007 | −4.6823 | NRLN1 | 0.356878935 | 0.525822669 | 0.528249548 |
| 225243_s_at | AB046821.1 | Hs.4007 | 0.0007 | −4.6812 | SLMAP | 0.554213615 | 0.739011846 | 0.700171981 |
| 224811_at | BF112093 | Hs.5724 | 0.0007 | −4.6687 | IMAGE: 5286019 | 0.466515157 | 0.725388678 | 0.638970142 |
| 212510_at | AA135522 | Hs.82432 | 0.0007 | −4.6621 | KIAA0089 | 0.605080242 | 0.73255191 | 0.802961174 |
| 218694_at | NM_016608.1 | Hs.9728 | 0.0007 | −4.6374 | ALEX1 | 0.602846403 | 0.707313012 | 0.772724682 |
| 203851_at | NM_002178.1 | Hs.274313 | 0.0007 | −4.6139 | IGFBP6 | 0.430883315 | 0.74596986 | 0.698725182 |
| 208848_at | M30471.1 | Hs.78989 | 0.0008 | −4.6038 | ADH5 | 0.663568149 | 0.777969527 | 0.908558621 |
| 203945_at | NM_001172.2 | Hs.172851 | 0.0008 | −4.5889 | ARG2 | 0.655767602 | 0.814139416 | 1.070857995 |
| 218717_s_at | NM_018192.1 | Hs.42824 | 0.0008 | −4.582 | MLAT4 | 0.491323587 | 0.719755368 | 1.063083603 |
| 203789_s_at | NM_006379.1 | Hs.171921 | 0.0008 | −4.5809 | SEMA3C | 0.41407478 | 0.713966234 | 0.812832558 |
| 212509_s_at | BF968134 | Hs.356623 | 0.0008 | −4.5787 | FLJ46603 | 0.389142337 | 0.624615411 | 0.532162455 |
| 205383_s_at | NM_015642.1 | Hs.159456 | 0.0008 | −4.5747 | ZNF288 | 0.548989134 | 0.694480542 | 0.641379066 |
| 207836_s_at | NM_006867.1 | Hs.80248 | 0.0009 | −4.5315 | RBPMS | 0.615089794 | 0.728032204 | 0.641435394 |
| 212361_s_at | AK000300.1 | Hs.374535 | 0.0009 | −4.5291 | ATP2A2 | 0.560457216 | 0.695746344 | 0.672952848 |
| 201841_s_at | NM_001540.2 | Hs.76067 | 0.0009 | −4.5208 | HSPB1 | 0.417356832 | 0.688393006 | 0.652979705 |
| 231098_at | BF939996 | Hs.10263 | 0.0009 | −4.5188 | IMAGE: 3439264 | 0.634015979 | 0.834876525 | 0.877772325 |
| 208637_x_at | BC003576.1 | Hs.119000 | 0.0009 | −4.5141 | ACTN1 | 0.507507171 | 0.670744352 | 0.696527754 |
| 203780_at | AF275945.1 | Hs.116651 | 0.0009 | −4.488 | EVA1 | 0.584182656 | 0.691457443 | 0.722126066 |
| 224710_at | AF322067.1 | Hs.301853 | 0.001 | −4.4671 | RAB34 | 0.603159118 | 0.718491133 | 0.652709312 |
| 205827_at | NM_000729.2 | Hs.80247 | 0.001 | −4.462 | CCK | 0.553054062 | 0.583055181 | 0.642464516 |
| 209747_at | J03241.1 | Hs.2025 | 0.001 | −4.449 | TGFB3 | 0.651515999 | 0.724745281 | 0.705691493 |
| 202948_at | NM_000877.1 | Hs.82112 | 0.001 | −4.4472 | IL1R1 | 0.604437089 | 0.82106783 | 1.181763499 |
| 227719_at | AA934610 | Hs.103262 | 0.001 | −4.4124 | MADH9 | 0.578200978 | 0.986277084 | 0.947599385 |
| 205413_at | NM_001584.1 | Hs.46638 | 0.001 | −4.4076 | C11ORF8 | 0.575640879 | 0.704424248 | 0.969192324 |
| 205158_at | NM_002937.1 | Hs.283749 | 0.0011 | −4.3995 | RNASE4 | 0.553261747 | 0.725854518 | 0.920722712 |
| 218094_s_at | NM_018478.1 | Hs.256086 | 0.0011 | −4.3978 | C20ORF35 | 0.634327286 | 0.733681563 | 0.668763089 |
| 227183_at | AI417267 | Hs.84630 | 0.0011 | −4.3909 | FLJ36638 | 0.476507931 | 0.748959021 | 0.510943793 |
| 200795_at | NM_004684.1 | Hs.75445 | 0.0012 | −4.3223 | SPARCL1 | 0.332891488 | 0.572497655 | 0.580836191 |
| 201289_at | NM_001554.1 | Hs.8867 | 0.0013 | −4.2923 | CYR61 | 0.357935903 | 0.675898639 | 0.504255247 |
| 209309_at | D90427.1 | Hs.71 | 0.0013 | −4.2714 | AZGP1 | 0.188868426 | 0.411500713 | 1.225895651 |
| 233496_s_at | AV726166 | Hs.180141 | 0.0013 | −4.2675 | CFL2 | 0.668714724 | 0.774968364 | 0.753424733 |
| 219295_s_at | NM_013363.1 | Hs.8944 | 0.0013 | −4.2607 | PCOLCE2 | 0.597237277 | 0.864177696 | 0.815426915 |
| 213110_s_at | AW052179 | Hs.169825 | 0.0013 | −4.2602 | COL4A5 | 0.623714985 | 0.82101802 | 0.725098366 |
| 208937_s_at | D13889.1 | Hs.75424 | 0.0014 | −4.2327 | ID1 | 0.340094789 | 0.424134354 | 0.368659343 |
| 208873_s_at | BC000232.1 | Hs.178112 | 0.0014 | −4.2192 | DP1 | 0.648135188 | 0.856221541 | 1.050337148 |
| 217728_at | NM_014624.2 | Hs.275243 | 0.0014 | −4.2167 | S100A6 | 0.485193905 | 0.623702181 | 0.541296022 |
| 221814_at | BF511315 | Hs.17270 | 0.0015 | −4.2012 | GPR124 | 0.621857706 | 0.752341694 | 0.704499619 |
| 217546_at | R06655 | Hs.188518 | 0.0015 | −4.1962 | MT1K | 0.456798259 | 0.504132777 | 0.901930375 |
| 232332_at | AI610999 | Hs.97594 | 0.0015 | −4.196 | KIAA1210 | 0.563855803 | 0.627364514 | 0.635441044 |
| 201234_at | NM_004517.1 | Hs.6196 | 0.0015 | −4.1911 | ILK | 0.603354892 | 0.6840541 | 0.683440877 |
| 232541_at | AK000106.1 | Hs.272227 | 0.0015 | −4.1859 | FLJ20099 | 0.552914557 | 0.849544303 | 0.615331046 |
| 225464_at | N30138 | Hs.250705 | 0.0015 | −4.1857 | C14ORF31 | 0.5944659 | 0.681084121 | 0.654445794 |
| 214898_x_at | AB038783.1 | Hs.129782 | 0.0016 | −4.1732 | MUC3B | 0.667579274 | 0.73585261 | 0.758074809 |
| 212423_at | AL049949.1 | Hs.28264 | 0.0016 | −4.1669 | FLJ90798 | 0.638894251 | 0.777384156 | 0.769528281 |
| 218552_at | NM_018281.1 | Hs.34579 | 0.0016 | −4.1514 | FLJ10948 | 0.588253779 | 0.87834189 | 0.833885251 |
| 209505_at | AI951185 | Hs.374991 | 0.0016 | −4.1505 | NR2F1 | 0.549274414 | 0.855084544 | 0.763129922 |
| 213338_at | BF062629 | Hs.35861 | 0.0016 | −4.1476 | RIS1 | 0.522606426 | 0.648514993 | 0.736649186 |
| 201389_at | NM_002205.1 | Hs.149609 | 0.0016 | −4.1416 | ITGA5 | 0.606773347 | 0.600410887 | 0.58991654 |
| 209288_s_at | AL136842.1 | Hs.260024 | 0.0016 | −4.1414 | CDC42EP3 | 0.477391739 | 0.66604325 | 0.682947642 |
| 221958_s_at | AA775681 | Hs.250746 | 0.0017 | −4.1363 | FLJ23091 | 0.63702265 | 0.874469966 | 1.118857498 |
| 209351_at | BC002690.1 | Hs.355214 | 0.0018 | −4.095 | KRT14 | 0.411699514 | 0.433050412 | 0.549270807 |
| 208949_s_at | BC001120.1 | Hs.621 | 0.0019 | −4.0458 | LGALS3 | 0.428078808 | 0.526116633 | 0.636966353 |
| 232224_at | AI274095 | Hs.356082 | 0.0019 | −4.0433 | MASP1 | 0.648107552 | 0.770747674 | 0.817503851 |
| 217168_s_at | AF217990.1 | Hs.146393 | 0.002 | −4.0353 | HERPUD1 | 0.582877469 | 0.698372654 | 1.125172106 |
| 213005_s_at | D79994.1 | Hs.77546 | 0.002 | −4.0149 | KANK | 0.585757723 | 0.687948638 | 0.739770133 |
| 227623_at | H16409 | Hs.298258 | 0.002 | −4.0108 | FLJ30478 | 0.599171183 | 0.685627452 | 0.729463584 |
| 204464_s_at | NM_001957.1 | Hs.76252 | 0.0022 | −3.9793 | EDNRA | 0.513268454 | 0.714259369 | 0.624579225 |
| 201300_s_at | NM_000311.1 | Hs.74621 | 0.0023 | −3.9405 | PRNP | 0.506550021 | 0.673224331 | 0.718988125 |
| 226051_at | BF973568 | Hs.55940 | 0.0023 | −3.9309 | SELM | 0.502400452 | 0.679612919 | 0.613157831 |
| 228325_at | AI363213 | Hs.278634 | 0.0024 | −3.9299 | KIAA0146 | 0.536626452 | 0.659648909 | 0.672068485 |
| 235518_at | AI741439 | Hs.144465 | 0.0024 | −3.9297 | SLC8A1 | 0.639765337 | 0.838297436 | 0.79588328 |
| 212848_s_at | BG036668 | Hs.334790 | 0.0024 | −3.9225 | FLJ14675 | 0.582906821 | 0.78306189 | 0.629500001 |
| 217023_x_at | AF099143 | — | 0.0025 | −3.904 | TPSB2 | 0.630895637 | 0.769488455 | 0.921618372 |
| 230577_at | AW014022 | Hs.170953 | 0.0026 | −3.8775 | sp: P00722 | 0.53651314 | 0.596534666 | 0.865585113 |
| 201645_at | NM_002160.1 | Hs.289114 | 0.0028 | −3.838 | TNC | 0.604361212 | 0.673498683 | 0.665240809 |
| 212805_at | AB002365.1 | Hs.23311 | 0.003 | −3.796 | KIAA0367 | 0.488940651 | 0.733752548 | 0.939729963 |
| 212993_at | AA114166 | Hs.381190 | 0.003 | −3.791 | IMAGE: 5311129 | 0.648379666 | 0.750751439 | 0.830305196 |
| 201121_s_at | NM_006667.2 | Hs.90061 | 0.003 | −3.7858 | PGRMC1 | 0.63646248 | 0.694566848 | 0.718897767 |
| 235759_at | AI095542 | Hs.302754 | 0.0031 | −3.7703 | EFCBP1 | 0.671683695 | 0.766080043 | 0.773001887 |
| 201667_at | NM_000165.2 | Hs.74471 | 0.0031 | −3.7625 | GJA1 | 0.38086039 | 0.477853618 | 0.510113877 |
| 206070_s_at | AF213459.1 | Hs.123642 | 0.0031 | −3.761 | EPHA3 | 0.578192384 | 1.028434338 | 0.942403658 |
| 209498_at | X16354.1 | Hs.50964 | 0.0032 | −3.7594 | CEACAM1 | 0.598189696 | 0.639236175 | 0.72565747 |
| 222325_at | AW974812 | Hs.433049 | 0.0033 | −3.7351 | EST386917 | 0.581645323 | 0.89684438 | 0.711318846 |
| 203973_s_at | NM_005195.1 | Hs.76722 | 0.0033 | −3.7327 | KIAA0146 | 0.340744017 | 0.4823812 | 0.484630011 |
| 206714_at | NM_001141.1 | Hs.111256 | 0.0034 | −3.7184 | ALOX15B | 0.456757922 | 0.654700344 | 1.510641843 |
| 202729_s_at | NM_000627.1 | Hs.241257 | 0.0034 | −3.712 | LTBP1 | 0.577127404 | 0.865778815 | 0.736276457 |
| 39248_at | N74607 | Hs.234642 | 0.0036 | −3.6776 | AQP3 | 0.442587059 | 0.573536836 | 0.776848921 |
| 204457_s_at | NM_002048.1 | Hs.65029 | 0.0037 | −3.6673 | GAS1 | 0.426786728 | 0.533346658 | 0.543269274 |
| 204971_at | NM_005213.1 | Hs.2621 | 0.0037 | −3.662 | CSTA | 0.637757056 | 0.642734275 | 0.649581736 |
| 204284_at | N26005 | Hs.303090 | 0.004 | −3.6304 | PPP1R3C | 0.595267584 | 0.676600675 | 0.692781509 |
| 202688_at | NM_003810.1 | Hs.83429 | 0.0041 | −3.6139 | TNFSF10 | 0.45407484 | 0.594718895 | 1.062889226 |
| 227917_at | AW192692 | Hs.169160 | 0.0041 | −3.6032 | DKFZp434N2116 | 0.664188052 | 0.871669924 | 0.737876071 |
| 201012_at | NM_000700.1 | Hs.78225 | 0.0043 | −3.5822 | ANXA1 | 0.464357655 | 0.611049645 | 0.481595141 |
| 203824_at | NM_004616.1 | Hs.84072 | 0.0043 | −3.5777 | TM4SF3 | 0.41872351 | 0.762172912 | 1.070782355 |
| 209540_at | NM_000618.1 | Hs.85112 | 0.0043 | −3.5768 | IGF1 | 0.604834335 | 0.931257424 | 0.877063322 |
| 226250_at | AA058578 | Hs.104627 | 0.0044 | −3.5722 | FLJ10158 | 0.593260939 | 0.75021829 | 0.684919925 |
| 222294_s_at | AW971415 | Hs.432533 | 0.0046 | −3.5408 | RAB27A | 0.65139431 | 0.878147649 | 1.479261234 |
| 218224_at | NM_006029.2 | Hs.194709 | 0.0047 | −3.5309 | PNMA1 | 0.569284754 | 0.703621182 | 0.725886997 |
| 241918_at | AI299378 | Hs.351615 | 0.0047 | −3.5304 | PCANAP5 | 0.593365377 | 0.807994275 | 1.030091863 |
| 209191_at | BC002654.1 | Hs.274398 | 0.0049 | −3.5095 | TUBB-5 | 0.576197173 | 0.641975742 | 0.599348386 |
| 228728_at | BF724137 | Hs.255416 | 0.0049 | −3.5031 | FLJ21986 | 0.633648453 | 0.823222679 | 0.751461991 |
| 235666_at | AA903473 | Hs.153717 | 0.005 | −3.5018 | sp: P39194 | 0.613016934 | 0.857437395 | 0.832762402 |
| 235094_at | AI972661 | Hs.30627 | 0.005 | −3.5004 | TPM4 | 0.455653643 | 0.860778088 | 0.495363995 |
| 203717_at | NM_001935.1 | Hs.44926 | 0.0051 | −3.4888 | DPP4 | 0.488633773 | 0.709272821 | 1.20340692 |
| 212185_x_at | NM_005953.1 | Hs.118786 | 0.0051 | −3.4834 | MT2A | 0.458542813 | 0.40997157 | 0.701563388 |
| 204908_s_at | NM_005178.1 | Hs.31210 | 0.0051 | −3.4813 | BCL3 | 0.644252573 | 0.665017966 | 0.71101296 |
| 202037_s_at | NM_003012.2 | Hs.7306 | 0.0052 | −3.4795 | SFRP1 | 0.542482197 | 0.861819298 | 0.687121176 |
| 203881_s_at | NM_004010.1 | Hs.169470 | 0.0052 | −3.4791 | DMD | 0.578897468 | 0.680754017 | 0.674303926 |
| 204326_x_at | NM_002450.1 | Hs.380778 | 0.0052 | −3.4728 | MT1X | 0.448212734 | 0.386428777 | 0.735631918 |
| 202289_s_at | NM_006997.1 | Hs.272023 | 0.0053 | −3.4667 | TACC2 | 0.644209586 | 0.844559734 | 1.054515739 |
| 225381_at | AW162210 | Hs.98518 | 0.0053 | −3.4651 | DKFZp686J24156 | 0.60032367 | 0.830881356 | 0.697291406 |
| 202133_at | AA081084 | Hs.24341 | 0.0053 | −3.4604 | TAZ | 0.596087848 | 0.789915793 | 0.767893734 |
| 200799_at | NM_005345.3 | Hs.75452 | 0.0055 | −3.4455 | HSPA1A | 0.525257873 | 1.022608345 | 1.350473323 |
| 225105_at | BF969397 | Hs.301711 | 0.0055 | −3.4396 | LOC387882 | 0.607521675 | 0.734980308 | 0.617862671 |
| 207935_s_at | NM_002274.1 | Hs.74070 | 0.0058 | −3.4118 | KRT13 | 0.608310078 | 0.789853708 | 0.656618334 |
| 227121_at | AL110204.1 | Hs.193784 | 0.006 | −3.3932 | DKFZp586K1922 | 0.595822645 | 0.75964906 | 0.71784473 |
| 204345_at | NM_001856.1 | Hs.26208 | 0.0061 | −3.3833 | COL16A1 | 0.609363288 | 0.888996822 | 0.619263593 |
| 213156_at | AL049423.1 | Hs.16193 | 0.0061 | −3.3813 | DKFZp586B211 | 0.614484055 | 0.79993889 | 0.90223163 |
| 221935_s_at | AK023140.1 | Hs.5997 | 0.0063 | −3.369 | MGC34132 | 0.657690674 | 0.784246268 | 0.706166103 |
| 203706_s_at | NM_003507.1 | Hs.173859 | 0.0063 | −3.3617 | FZD7 | 0.556884887 | 0.743584877 | 0.691777229 |
| 204793_at | NM_014710.1 | Hs.113082 | 0.0064 | −3.3542 | GASP | 0.640999038 | 0.770150708 | 0.676227311 |
| 203708_at | NM_002600.1 | Hs.188 | 0.0065 | −3.3514 | PDE4B | 0.618721093 | 0.695543706 | 0.740177755 |
| 212859_x_at | BF217861 | — | 0.0065 | −3.3489 | MT1E | 0.431199359 | 0.381553146 | 0.798187882 |
| 204537_s_at | NM_004961.2 | Hs.22785 | 0.0066 | −3.3377 | GABRE | 0.603828317 | 0.694224314 | 0.579239977 |
| 202888_s_at | NM_001150.1 | Hs.1239 | 0.0067 | −3.3349 | ANPEP | 0.370164997 | 0.477411102 | 1.562801826 |
| 202391_at | NM_006317.1 | Hs.79516 | 0.0069 | −3.3147 | BASP1 | 0.463230986 | 0.909162083 | 0.838497202 |
| 204748_at | NM_000963.1 | Hs.196384 | 0.0069 | −3.3147 | PTGS2 | 0.391552844 | 0.610499324 | 0.522728242 |
| 223557_s_at | AB017269.1 | Hs.22791 | 0.0072 | −3.2939 | TMEFF2 | 0.478486722 | 2.173964939 | 5.040357989 |
| 222303_at | AV700891 | Hs.292477 | 0.0072 | −3.2925 | ETS2 | 0.500190086 | 0.644047093 | 0.477238473 |
| 211456_x_at | AF333388.1 | Hs.367850 | 0.0073 | −3.2809 | MT1H | 0.573088114 | 0.512423936 | 0.790642019 |
| 214696_at | AF070569.1 | Hs.417157 | 0.0074 | −3.2775 | MGC14376 | 0.500101466 | 0.644862395 | 0.54026883 |
| 201599_at | NM_000274.1 | Hs.75485 | 0.0074 | −3.2775 | OAT | 0.560449825 | 0.628852944 | 0.653941647 |
| 218731_s_at | NM_022834.1 | Hs.110443 | 0.0076 | −3.2575 | FLJ22215 | 0.647897719 | 0.731950802 | 0.805715513 |
| 228188_at | AI860150 | Hs.5890 | 0.0078 | −3.2486 | FLJ23306 | 0.612483767 | 0.730400346 | 0.657667139 |
| 212914_at | AV648364 | Hs.356416 | 0.0079 | −3.2399 | CBX7 | 0.672491781 | 0.780716904 | 0.690054773 |
| 200696_s_at | NM_000177.1 | Hs.290070 | 0.008 | −3.2335 | GSN | 0.483261114 | 0.725938182 | 0.568269871 |
| 206211_at | NM_000450.1 | Hs.89546 | 0.0083 | −3.2081 | SELE | 0.490034502 | 0.703663072 | 0.738701475 |
| 242736_at | AI377221 | Hs.40528 | 0.0084 | −3.2052 | IMAGE: 2064065 | 0.602976013 | 0.807016023 | 0.621771592 |
| 221024_s_at | NM_030777.1 | Hs.305971 | 0.0084 | −3.2046 | SLC2A10 | 0.639798214 | 0.925382652 | 1.45314006 |
| 205229_s_at | AA669336 | Hs.21016 | 0.0085 | −3.1955 | COCH | 0.620495813 | 0.854818559 | 0.735661252 |
| 211965_at | X79067.1 | Hs.85155 | 0.0086 | −3.1932 | ZFP36L1 | 0.644547553 | 0.774491249 | 0.800099031 |
| 201560_at | NM_013943.1 | Hs.25035 | 0.0086 | −3.1884 | CLIC4 | 0.628588945 | 0.799632703 | 0.709436844 |
| 202018_s_at | NM_002343.1 | Hs.105938 | 0.0087 | −3.1816 | LTF | 0.0970549 | 0.17189767 | 0.307421109 |
| 201360_at | NM_000099.1 | Hs.304682 | 0.009 | −3.1674 | CST3 | 0.598218982 | 0.683984155 | 0.80851963 |
| 201369_s_at | NM_006887.1 | Hs.78909 | 0.009 | −3.1669 | ZFP36L2 | 0.57332007 | 0.695638926 | 0.581983214 |
| 225442_at | AI799915 | Hs.349303 | 0.0091 | −3.16 | DDR2 | 0.650022328 | 0.851998744 | 0.703655507 |
| 212724_at | BG054844 | Hs.6838 | 0.0094 | −3.138 | ARHE | 0.524405985 | 0.610187469 | 0.578512935 |
| 202336_s_at | NM_000919.1 | Hs.83920 | 0.0097 | −3.1204 | PAM | 0.560777596 | 1.000931184 | 0.831990839 |
| 226189_at | BF513121 | Hs.367688 | 0.0099 | −3.1117 | IMAGE: 4794726 | 0.628864888 | 0.787069309 | 0.733048653 |
| 221872_at | AI669229 | Hs.82547 | 0.01 | −3.1039 | RARRES1 | 0.33062532 | 0.489452465 | 0.499917103 |
| 212761_at | AI703074 | Hs.348412 | 0.0102 | −3.0937 | TCF7L2 | 0.625047654 | 0.858457558 | 0.920807486 |
| 243296_at | AA873350 | Hs.176554 | 0.0106 | −3.0756 | PBEF | 0.337927134 | 0.595396083 | 0.402394619 |
| 241897_at | AA491949 | Hs.409080 | 0.0108 | −3.0635 | CRL2 precusor | 0.628387896 | 0.855940324 | 0.600396555 |
| 212099_at | AI263909 | Hs.204354 | 0.0112 | −3.0404 | ARHB | 0.402558963 | 0.5374298 | 0.46564017 |
| 225876_at | T84558 | Hs.13804 | 0.0113 | −3.0358 | DJ462O23.2 | 0.526611323 | 0.650766767 | 0.893799448 |
| 201041_s_at | NM_004417.2 | Hs.171695 | 0.0116 | −3.0239 | DUSP1 | 0.451274478 | 0.665471417 | 0.688731099 |
| 226252_at | AA058578 | Hs.104627 | 0.0116 | −3.023 | FLJ10158 | 0.659463151 | 0.790315933 | 0.809873125 |
| 230788_at | BF059748 | Hs.421105 | 0.0116 | −3.0217 | GCNT2 | 0.511752041 | 0.591273522 | 0.882837241 |
| 200953_s_at | NM_001759.1 | Hs.75586 | 0.0118 | −3.0149 | CCND2 | 0.581793396 | 0.760195445 | 0.718824623 |
| 33323_r_at | X57348 | Hs.184510 | 0.0118 | −3.0142 | SFN | 0.432853115 | 0.578204169 | 0.833345335 |
| 204745_x_at | NM_005950.1 | Hs.433391 | 0.0121 | −3.0012 | MT1G | 0.456465598 | 0.425042163 | 0.791028837 |
| 201150_s_at | NM_000362.2 | Hs.245188 | 0.0121 | −3.0004 | TIMP3 | 0.615278264 | 0.677143574 | 0.709474175 |
| 222162_s_at | AK023795.1 | Hs.8230 | 0.0121 | −2.9969 | ADAMTS1 | 0.417960532 | 0.68593523 | 0.555010188 |
| 213275_x_at | BE875786 | Hs.297939 | 0.0122 | −2.9946 | CTSB | 0.639593717 | 0.761818881 | 0.730652349 |
| 219682_s_at | NM_016569.1 | Hs.267182 | 0.0124 | −2.9839 | TBX3 | 0.523809912 | 0.886022121 | 0.970469152 |
| 238481_at | AW512787 | Hs.404077 | 0.0125 | −2.9807 | MGP | 0.606083743 | 1.138279606 | 0.670651525 |
| 209656_s_at | AL136550.1 | Hs.8769 | 0.0128 | −2.9684 | TM4SF10 | 0.560601819 | 0.899717295 | 0.757505615 |
| 201464_x_at | BG491844 | Hs.78465 | 0.013 | −2.9584 | JUN | 0.534670849 | 0.843913283 | 0.892066246 |
| 202350_s_at | NM_002380.2 | Hs.19368 | 0.0132 | −2.9515 | MATN2 | 0.595033679 | 0.834264276 | 0.795741335 |
| 212768_s_at | AL390736 | Hs.273321 | 0.0133 | −2.9456 | GW112 | 0.225216833 | 0.436827315 | 0.393985727 |
| 209156_s_at | AY029208.1 | Hs.159263 | 0.0133 | −2.9454 | COL6A2 | 0.486933097 | 0.608880847 | 0.450512965 |
| 205692_s_at | NM_001775.1 | Hs.66052 | 0.0134 | −2.9417 | CD38 | 0.615350798 | 0.658995924 | 0.989624421 |
| 222722_at | AV700059 | Hs.109439 | 0.0136 | −2.9337 | OGN | 0.545423692 | 0.806415801 | 0.715131507 |
| 209016_s_at | BC002700.1 | Hs.23881 | 0.014 | −2.9156 | KRT7 | 0.642306014 | 0.74588737 | 0.690949593 |
| 215111_s_at | AK027071.1 | Hs.114360 | 0.0141 | −2.9136 | TSC22 | 0.497282694 | 0.531538699 | 0.6436215 |
| 209621_s_at | AF002280.1 | Hs.135281 | 0.0142 | −2.9109 | ALP | 0.59333833 | 0.703856749 | 0.680927442 |
| 242868_at | T70087 | Hs.307559 | 0.0143 | −2.9076 | IMAGE: 80996 | 0.570499373 | 0.720976952 | 0.548770053 |
| 218718_at | NM_016205.1 | Hs.43080 | 0.0145 | −2.8967 | PDGFC | 0.570589136 | 0.759913242 | 0.671837954 |
| 200884_at | NM_001823.1 | Hs.173724 | 0.0145 | −2.8963 | CKB | 0.509732177 | 0.678228409 | 0.844919959 |
| 212089_at | M13452.1 | Hs.377973 | 0.0152 | −2.8724 | LMNA | 0.665116105 | 0.739568287 | 0.679437588 |
| 202672_s_at | NM_001674.1 | Hs.460 | 0.0152 | −2.8699 | ATF3 | 0.254053258 | 0.577524204 | 0.42844299 |
| 216598_s_at | S69738.1 | Hs.303649 | 0.0153 | −2.8667 | CCL2 | 0.441821303 | 0.464466134 | 0.409043457 |
| 226769_at | AI802391 | Hs.32478 | 0.0154 | −2.8649 | LOC387758 | 0.643967758 | 1.0013538 | 0.839964674 |
| 209189_at | BC004490.1 | Hs.25647 | 0.0158 | −2.8487 | FOS | 0.329749759 | 0.628331868 | 0.493449262 |
| 202286_s_at | J04152 | Hs.23582 | 0.0159 | −2.8462 | TACSTD2 | 0.31642776 | 0.625542647 | 1.021260519 |
| 225673_at | BE908995 | Hs.380906 | 0.0161 | −2.8386 | LOC91663 | 0.566986589 | 0.675313081 | 0.623314519 |
| 205862_at | NM_014668.1 | Hs.193914 | 0.0165 | −2.8242 | GREB1 | 0.506078166 | 0.943886011 | 1.380149032 |
| 205225_at | NM_000125.1 | Hs.1657 | 0.0167 | −2.819 | ESR1 | 0.51712671 | 0.924139409 | 0.697838254 |
| 231783_at | AI500293 | Hs.247917 | 0.0174 | −2.7963 | CHRM1 | 0.641574237 | 0.764137428 | 1.312516824 |
| 201694_s_at | NM_001964.1 | Hs.326035 | 0.0174 | −2.7957 | EGR1 | 0.39646573 | 0.679207349 | 0.566237865 |
| 213428_s_at | AA92373 | Hs.108885 | 0.0177 | −2.7862 | COL6A1 | 0.56253883 | 0.690206606 | 0.489695051 |
| 209369_at | M63310.1 | Hs.1378 | 0.0182 | −2.7707 | ANXA3 | 0.643888077 | 0.907333193 | 1.231309972 |
| 224894_at | BF210049 | Hs.84520 | 0.0184 | −2.7634 | YAP1 | 0.607783703 | 0.821687742 | 0.748843462 |
| 208763_s_at | AL110191.1 | Hs.75450 | 0.0185 | −2.7619 | DSIPI | 0.610365851 | 0.729534861 | 0.802532704 |
| 244239_at | AI887306 | Hs.137221 | 0.0194 | −2.7355 | YN63H06 | 0.618590896 | 0.795484734 | 0.676415916 |
| 201425_at | NM_000690.1 | Hs.195432 | 0.0199 | −2.7205 | ALDH2 | 0.64506947 | 0.71496059 | 0.871943306 |
| 217165_x_at | M10943 | Hs.381097 | 0.0199 | −2.7204 | MT1F | 0.532277831 | 0.459410851 | 0.95574968 |
| 201531_at | NM_003407.1 | Hs.343586 | 0.0201 | −2.7164 | ZFP36 | 0.368822278 | 0.573326486 | 0.51833161 |
| 201236_s_at | NM_006763.1 | Hs.75462 | 0.0202 | −2.7111 | BTG2 | 0.449196974 | 0.574666196 | 0.564492749 |
| 225945_at | BF219240 | Hs.115659 | 0.0204 | −2.7073 | VIK | 0.63857255 | 0.692757333 | 0.701380412 |
| 202489_s_at | BC005238.1 | Hs.301350 | 0.0205 | −2.705 | FXYD3 | 0.413544476 | 0.691155271 | 1.267793962 |
| 204719_at | NM_007168.1 | Hs.38095 | 0.0209 | −2.693 | ABCA8 | 0.565139968 | 0.757214801 | 0.707955742 |
| 217967_s_at | AF288391.1 | Hs.48778 | 0.0209 | −2.6929 | C1ORF24 | 0.543959386 | 0.73063063 | 1.104433103 |
| 215078_at | AL050388.1 | Hs.372783 | 0.0211 | −2.687 | SOD2 | 0.647668168 | 0.732598208 | 0.703135648 |
| 225557_at | AI091372 | Hs.6607 | 0.0212 | −2.6843 | AXUD1 | 0.53852929 | 0.664192806 | 0.633086763 |
| 204259_at | NM_002423.2 | Hs.2256 | 0.0215 | −2.6775 | MMP7 | 0.450118957 | 0.7288099 | 0.768253699 |
| 205960_at | NM_002612.1 | Hs.8364 | 0.0215 | −2.6766 | PDK4 | 0.609608362 | 0.706936283 | 0.617091029 |
| 209210_s_at | Z24725.1 | Hs.75260 | 0.0219 | −2.6683 | PLEKHC1 | 0.549014436 | 0.638717949 | 0.609727499 |
| 209101_at | M92934.1 | Hs.75511 | 0.0223 | −2.6578 | CTGF | 0.451024698 | 0.732153169 | 0.510263768 |
| 226506_at | AI742570 | Hs.380149 | 0.0223 | −2.6567 | FLJ13710 | 0.659953836 | 0.709491486 | 0.758949079 |
| 209118_s_at | AF141347.1 | Hs.433394 | 0.0232 | −2.6349 | TUBA3 | 0.668082045 | 0.768266303 | 0.670094444 |
| 213791_at | NM_006211.1 | Hs.93557 | 0.0237 | −2.6238 | PENK | 0.649165182 | 0.735398814 | 0.732302884 |
| 212230_at | AL576654 | — | 0.024 | −2.6149 | PPAP2B | 0.548857227 | 0.589286375 | 0.61198091 |
| 217744_s_at | NM_022121.1 | Hs.303125 | 0.0242 | −2.6111 | PIGPC1 | 0.636297335 | 0.789650873 | 0.957541661 |
| 201005_at | NM_001769.1 | Hs.1244 | 0.0245 | −2.605 | CD9 | 0.471999699 | 0.789958319 | 1.068501023 |
| 227399_at | AI754423 | Hs.367211 | 0.0251 | −2.5903 | LOC51159 | 0.56959877 | 0.943253306 | 1.140816664 |
| 237077_at | AI821895 | Hs.433060 | 0.0254 | −2.5844 | IMAGE: 1203949 | 0.585987134 | 0.846219403 | 0.980927952 |
| 202340_x_at | NM_002135.1 | Hs.1119 | 0.0264 | −2.5621 | NR4A1 | 0.348025216 | 0.674634071 | 0.50042662 |
| 203140_at | NM_001706.1 | Hs.155024 | 0.0265 | −2.5597 | BCL6 | 0.653995843 | 0.755613259 | 0.672169483 |
| 227642_at | AI928242 | Hs.119903 | 0.0266 | −2.5575 | TFCP2L1 | 0.641596799 | 0.73268621 | 0.668940723 |
| 213931_at | AI819238 | Hs.180919 | 0.0282 | −2.5249 | pir: A40227 | 0.629101722 | 0.781558812 | 0.616683305 |
| 217775_s_at | NM_016026.1 | Hs.179817 | 0.0286 | −2.5171 | RDH11 | 0.464165784 | 0.77978021 | 1.670415923 |
| 213564_x_at | BE042354 | Hs.234489 | 0.0289 | −2.5125 | LDHB | 0.487639647 | 0.60736074 | 0.629709594 |
| 201650_at | NM_002276.1 | Hs.182265 | 0.03 | −2.4907 | KRT19 | 0.556260378 | 0.552100901 | 0.58183457 |
| 209304_x_at | AF087853.1 | Hs.110571 | 0.0306 | −2.4802 | GADD45B | 0.527433735 | 0.667118834 | 0.580847272 |
| 243618_s_at | BF678830 | Hs.382367 | 0.0306 | −2.4797 | LOC152485 | 0.604180806 | 0.769951673 | 0.860931014 |
| 240221_at | AV704610 | Hs.318381 | 0.031 | −2.4725 | CSNK1A1 | 0.659752573 | 0.903938631 | 0.647440833 |
| 201105_at | NM_002305.2 | Hs.382367 | 0.0312 | −2.4686 | LGALS1 | 0.641063556 | 0.664405546 | 0.526293118 |
| 224917_at | BF674052 | Hs.374415 | 0.032 | −2.4542 | VMP1 | 0.417797614 | 0.725339183 | 0.407411034 |
| 222927_s_at | AW295812 | Hs.98927 | 0.032 | −2.454 | LMAN1L | 0.587807901 | 0.802616467 | 0.755345307 |
| 212665_at | AL556438 | Hs.12813 | 0.0323 | −2.4486 | DKFZP434J214 | 0.523667633 | 0.624272209 | 0.616181214 |
| 224755_at | BE621524 | Hs.8203 | 0.0326 | −2.4437 | SMBP | 0.648166532 | 0.885971012 | 0.980484508 |
| 201631_s_at | NM_003897.1 | Hs.76095 | 0.035 | −2.404 | IER3 | 0.511124962 | 0.534169945 | 0.466723395 |
| 221841_s_at | BF514079 | Hs.376206 | 0.0355 | −2.3961 | KLF4 | 0.444530205 | 0.685266095 | 0.582181416 |
| 212097_at | AU147399 | Hs.74034 | 0.0372 | −2.3686 | CAV1 | 0.672011287 | 0.525135392 | 0.575693007 |
| 207826_s_at | NM_002167.1 | Hs.76884 | 0.0374 | −2.3669 | ID3 | 0.66544141 | 0.686424697 | 0.588659692 |
| 36711_at | AL021977 | Hs.51305 | 0.0379 | −2.3589 | MAFF | 0.433687817 | 0.557218356 | 0.563652161 |
| 202720_at | NM_015641.1 | Hs.165986 | 0.0396 | −2.3343 | TES | 0.644177594 | 0.688210629 | 0.698168263 |
| 202768_at | NM_006732.1 | Hs.75678 | 0.0399 | −2.3293 | FOSB | 0.278626863 | 0.557553338 | 0.388079334 |
| 223218_s_at | AB037925.1 | Hs.301183 | 0.04 | −2.3274 | MAIL | 0.55298983 | 0.81241416 | 0.445748711 |
| 203962_s_at | NM_006393.1 | Hs.5025 | 0.0417 | −2.304 | NEBL | 0.66859378 | 0.788135019 | 0.747562737 |
| 212531_at | NM_005564.1 | Hs.204238 | 0.0428 | −2.2902 | LCN2 | 0.246089432 | 0.278320044 | 0.355266869 |
| 205251_at | NM_022817.1 | Hs.153405 | 0.0444 | −2.2687 | PER2 | 0.633196234 | 0.671066633 | 0.624644315 |
| 209184_s_at | BF700086 | Hs.143648 | 0.0453 | −2.2571 | IRS2 | 0.609218577 | 0.909010722 | 0.812757521 |
| 205319_at | NM_005672.1 | Hs.423634 | 0.0481 | −2.2232 | PSCA | 0.578225484 | 0.829291736 | 0.87744188 |
| 201312_s_at | NM_003022.1 | Hs.14368 | 0.0515 | −2.1839 | SH3BGRL | 0.552399851 | 0.754499178 | 0.836452923 |
| 205207_at | NM_000600.1 | Hs.93913 | 0.0523 | −2.1756 | IL6 | 0.593094851 | 0.684302598 | 0.592307215 |
| 206260_at | NM_003241.1 | Hs.2387 | 0.0524 | −2.1739 | TGM4 | 0.259043972 | 0.32178001 | 0.347372965 |
| 211753_s_at | BC005956.1 | Hs.105314 | 0.0525 | −2.1733 | RLN1 | 0.553157866 | 1.243044777 | 1.980477424 |
| 213503_x_at | BE908217 | Hs.217493 | 0.0527 | −2.1708 | ANXA2 | 0.635697023 | 0.542468458 | 0.54146373 |
| 225344_at | AL035689 | Hs.339283 | 0.053 | −2.1678 | NCOA7 | 0.496528879 | 0.530808955 | 0.416492601 |
| 203791_at | NM_005509.2 | Hs.181042 | 0.053 | −2.1677 | DMXL1 | 0.645400966 | 0.960835018 | 1.226258193 |
| 204351_at | NM_005980.1 | Hs.2962 | 0.0537 | −2.1596 | S100P | 0.49193707 | 0.496153624 | 0.601000645 |
| 201170_s_at | NM_003670.1 | Hs.171825 | 0.0546 | −2.1507 | BHLHB2 | 0.548460448 | 0.574865751 | 0.49210945 |
| 225046_at | BF667120 | Hs.406650 | 0.0546 | −2.1504 | FLJ41510 | 0.523155822 | 0.568607967 | 0.662068658 |
| 225612_s_at | BE672260 | Hs.136414 | 0.0573 | −2.1225 | B3GNT5 | 0.669623796 | 0.768179338 | 0.63246118 |
| 201473_at | NM_002229.1 | Hs.400124 | 0.0573 | −2.1224 | JUNB | 0.493732742 | 0.61851068 | 0.572322256 |
| 204582_s_at | NM_001648.1 | Hs.171995 | 0.0601 | −2.0949 | KLK3 | 0.283429406 | 0.589742134 | 1.304985589 |
| 212789_at | AI796581 | Hs.13421 | 0.0644 | −2.0552 | KIAA0056 | 0.608997484 | 0.939628975 | 1.410142531 |
| 203908_at | NM_003759.1 | Hs.5462 | 0.0649 | −2.0506 | SLC4A4 | 0.513131934 | 1.481621069 | 2.537853202 |
| 201563_at | L29008.1 | Hs.878 | 0.0654 | −2.046 | SORD | 0.451194273 | 0.861192916 | 1.594819444 |
| 203574_at | NM_005384.1 | Hs.79334 | 0.0695 | −2.0109 | NFIL3 | 0.565727477 | 0.577268422 | 0.650209608 |
| 206529_x_at | NM_000441.1 | Hs.159275 | 0.0704 | −2.0037 | SLC26A4 | 0.551951321 | 0.631352534 | 0.66982304 |
| 211298_s_at | AF116645.1 | Hs.184411 | 0.0708 | 2 | ALB | 4.038348409 | 1.02982235 | 1.072392767 |
| 222516_at | AA700485 | Hs.298442 | 0.0677 | 2.0259 | AP3M1 | 1.540043784 | 1.105426064 | 1.21683644 |
| 209160_at | AB018580.1 | Hs.78183 | 0.0674 | 2.0289 | AKR1C3 | 1.499988089 | 1.148809647 | 0.95052273 |
| 211110_s_at | AF162704.1 | Hs.99915 | 0.0668 | 2.0338 | AR | 1.963334407 | 1.317125468 | 1.5340528 |
| 200598_s_at | AI582238 | Hs.82689 | 0.0653 | 2.0467 | TRA1 | 1.52452446 | 1.27999211 | 1.989934304 |
| 201852_x_at | AI813758 | Hs.119571 | 0.0632 | 2.0658 | COL3A1 | 1.902896136 | 1.730098336 | 0.796575886 |
| 227235_at | AI758408 | Hs.22247 | 0.0619 | 2.0778 | FLJ42250 | 1.576454945 | 1.289772714 | 1.496714465 |
| 229530_at | BF002625 | Hs.29088 | 0.0617 | 2.0801 | IMAGE: 3315604 | 1.65327194 | 1.327584952 | 1.629400268 |
| 226884_at | N71874 | Hs.126085 | 0.0595 | 2.1008 | LRRN1 | 1.548535045 | 1.363318876 | 1.312256682 |
| 201008_s_at | NM_006472.1 | Hs.179526 | 0.0575 | 2.1211 | TXNIP | 1.799826636 | 1.161864435 | 1.552769217 |
| 226726_at | W63676 | Hs.356547 | 0.0544 | 2.1531 | LOC129642 | 1.703434777 | 1.376392585 | 1.615871928 |
| 223423_at | BC000181.2 | Hs.97101 | 0.054 | 2.1563 | GPCR1 | 1.764712506 | 1.80971944 | 2.088695561 |
| 217733_s_at | NM_021103.1 | Hs.76293 | 0.0503 | 2.1978 | TMSB10 | 1.503806522 | 1.109655695 | 1.077926843 |
| 216379_x_at | AK000168.1 | Hs.375108 | 0.0499 | 2.2026 | FLJ20161 | 1.825688217 | 1.303355294 | 1.586083962 |
| 213812_s_at | AK024748.1 | Hs.108708 | 0.0497 | 2.2039 | CAMKK2 | 1.647330039 | 1.856918875 | 2.401956042 |
| 211161_s_at | AF130082.1 | Hs.327412 | 0.0462 | 2.2467 | FLC1492 | 1.848041612 | 1.554130932 | 0.94132736 |
| 220161_s_at | NM_019114.1 | Hs.267997 | 0.0455 | 2.2553 | EPB41L4B | 1.512813189 | 1.488934601 | 1.573558969 |
| 225499_at | AW296194 | Hs.17235 | 0.0439 | 2.2758 | FLJ22541 | 1.620548305 | 1.466725395 | 1.475166509 |
| 227492_at | AI829721 | Hs.171952 | 0.0427 | 2.2904 | OCLN | 1.541582175 | 1.377461428 | 1.232178281 |
| 218350_s_at | NM_015895.1 | Hs.234896 | 0.0412 | 2.3115 | GMNN | 1.541471697 | 1.008334353 | 0.849756992 |
| 209613_s_at | M21692.1 | Hs.4 | 0.0408 | 2.3166 | ADH1B | 2.004916435 | 0.962435512 | 0.837725721 |
| 209374_s_at | BC001872.1 | Hs.153261 | 0.0393 | 2.3381 | IGHM | 1.816654151 | 1.305366845 | 1.032416003 |
| 226226_at | AI282982 | Hs.283552 | 0.0359 | 2.3898 | LOC120224 | 1.756061279 | 1.200620676 | 1.260631471 |
| 206351_s_at | NM_002617.1 | Hs.247220 | 0.0347 | 2.4093 | PEX10 | 1.622699512 | 1.27142138 | 1.489345755 |
| 211074_at | AF000381.1 | Hs.73769 | 0.0326 | 2.4444 | Folate binding protein | 1.578683325 | 1.381413609 | 1.789411263 |
| 202427_s_at | NM_015415.1 | Hs.76285 | 0.0323 | 2.4497 | DKFZP564B167 | 1.670183347 | 1.351905473 | 2.246923836 |
| 201720_s_at | AI589086 | Hs.79356 | 0.032 | 2.4552 | LAPTM5 | 1.69885847 | 1.061164515 | 0.966340129 |
| 227197_at | AI989530 | Hs.240845 | 0.0316 | 2.4606 | DKFZP434D146 | 1.659535166 | 1.978903297 | 2.278268404 |
| 221942_s_at | AI719730 | Hs.75295 | 0.0313 | 2.4669 | GUCY1A3 | 1.844715047 | 1.448858579 | 2.085521221 |
| 233950_at | AK000873.1 | Hs.151301 | 0.031 | 2.473 | CADPS | 1.546427503 | 1.085472457 | 0.984688555 |
| 217736_s_at | NM_014413.2 | Hs.258730 | 0.0303 | 2.4847 | HRI | 1.536515183 | 1.604502316 | 1.817901191 |
| 208808_s_at | BC000903.1 | Hs.80684 | 0.0295 | 2.501 | HMGB2 | 1.675010385 | 1.162704083 | 0.924389164 |
| 204319_s_at | NM_002925.2 | Hs.82280 | 0.0294 | 2.5022 | RGS10 | 1.541898982 | 1.309324255 | 1.795358401 |
| 203215_s_at | AA877789 | Hs.22564 | 0.0291 | 2.5082 | MYO6 | 1.633958411 | 1.606283969 | 1.861691317 |
| 202854_at | NM_000194.1 | Hs.82314 | 0.0289 | 2.5108 | HPRT1 | 1.529834801 | 1.179426162 | 1.174940245 |
| 202310_s_at | NM_000088.1 | Hs.172928 | 0.0287 | 2.5162 | COL1A1 | 2.033537613 | 1.914940615 | 0.772389958 |
| 206214_at | NM_005084.1 | Hs.93304 | 0.0285 | 2.519 | PLA2G7 | 1.605980146 | 1.707204536 | 1.777048436 |
| 217871_s_at | NM_002415.1 | Hs.73798 | 0.0283 | 2.5237 | MIF | 1.769625594 | 1.343349079 | 1.596049197 |
| 209424_s_at | NM_014324.1 | Hs.128749 | 0.0281 | 2.5272 | AMACR | 2.116938837 | 2.324343802 | 5.066327548 |
| 217848_s_at | NM_021129.1 | Hs.184011 | 0.0255 | 2.5829 | PP | 1.711672524 | 1.14995071 | 1.246624657 |
| 220199_s_at | NM_022831.1 | Hs.107637 | 0.0238 | 2.6218 | FLJ12806 | 2.391285989 | 1.145492807 | 1.121762377 |
| 208905_at | BC005299.1 | Hs.169248 | 0.022 | 2.6644 | CYCS | 1.570755038 | 1.345901439 | 1.3984069 |
| 224840_at | AL122066.1 | Hs.7557 | 0.0218 | 2.6687 | FKBP5 | 1.48846771 | 1.036856486 | 1.850099599 |
| 229152_at | AI718421 | Hs.320147 | 0.0216 | 2.6754 | C4ORF7 | 2.322871439 | 0.998617569 | 0.971594162 |
| 203431_s_at | NM_014715.1 | Hs.111138 | 0.0216 | 2.6762 | RICS | 1.52225145 | 1.312998897 | 1.230108289 |
| 205943_at | NM_005651.1 | Hs.183671 | 0.0209 | 2.6944 | TDO2 | 1.760600293 | 1.50100665 | 1.188986943 |
| 201422_at | NM_006332.1 | Hs.14623 | 0.0206 | 2.7003 | IFI30 | 1.552309296 | 1.136298126 | 0.932541939 |
| 218559_s_at | NM_005461.1 | Hs.169487 | 0.0205 | 2.704 | MAFB | 1.565093687 | 1.168516107 | 1.174192575 |
| 226880_at | AL035851 | Hs.118064 | 0.0198 | 2.7228 | NUCKS | 1.600299748 | 1.366839531 | 1.39888628 |
| 209875_s_at | M83248.1 | Hs.313 | 0.0196 | 2.729 | SPP1 | 1.778246021 | 1.51644862 | 1.275916329 |
| 226039_at | AW006441 | Hs.24210 | 0.0187 | 2.7549 | MGAT4A | 1.627101772 | 1.219058919 | 1.187042252 |
| 225647_s_at | AI246687 | Hs.10029 | 0.0185 | 2.7623 | CTSC | 1.501738811 | 1.165441402 | 1.098532931 |
| 224665_at | AK023981.1 | Hs.178485 | 0.0176 | 2.7906 | LOC119504 | 1.530272787 | 0.998417546 | 1.075123958 |
| 241926_s_at | AA296657 | Hs.45514 | 0.0174 | 2.7956 | ERG | 1.914432841 | 1.28776349 | 1.496429254 |
| 201288_at | NM_001175.1 | Hs.83656 | 0.0174 | 2.7963 | ARHGDIB | 1.83262893 | 1.014920395 | 1.014793823 |
| 229724_at | AI693153 | Hs.1440 | 0.0171 | 2.8068 | GABRB3 | 1.616657166 | 1.451776055 | 1.846212704 |
| 200644_at | NM_023009.1 | Hs.75061 | 0.0163 | 2.8315 | MLP | 1.960047156 | 1.934633141 | 2.382304727 |
| 200665_s_at | NM_003118.1 | Hs.111779 | 0.0158 | 2.8486 | SPARC | 1.839336794 | 1.422425643 | 0.906449465 |
| 224833_at | BE218980 | Hs.18063 | 0.0156 | 2.8564 | ETS1 | 1.769713096 | 1.01329137 | 0.985362417 |
| 204416_x_at | NM_001645.2 | Hs.268571 | 0.015 | 2.8784 | APOC1 | 2.659455722 | 1.314190401 | 1.206631876 |
| 218025_s_at | NM_006117.1 | Hs.15250 | 0.0148 | 2.8861 | PECI | 1.556592348 | 1.317497889 | 1.73958772 |
| 200771_at | NM_002293.2 | Hs.214982 | 0.0138 | 2.9251 | LAMC1 | 1.551677343 | 1.021886687 | 0.909481221 |
| 217294_s_at | U88968.1 | Hs.381397 | 0.0134 | 2.9417 | ENO1 | 1.709198983 | 1.094746038 | 1.239077599 |
| 227405_s_at | AW340311 | Hs.302634 | 0.0131 | 2.9538 | FZD8 | 1.554378677 | 1.078120743 | 1.146047942 |
| 203910_at | NM_004815.1 | Hs.70983 | 0.0129 | 2.965 | PARG1 | 1.566658602 | 1.091725294 | 1.196943379 |
| 209781_s_at | AF069681.1 | Hs.13565 | 0.0127 | 2.9699 | KHDRBS3 | 1.720661696 | 1.119899822 | 1.079578584 |
| 200971_s_at | NM_014445.1 | Hs.76698 | 0.0127 | 2.9726 | SERP1 | 1.559636173 | 1.331160738 | 1.628062522 |
| 226801_s_at | W72220 | Hs.107637 | 0.0123 | 2.9916 | FLJ12806 | 2.393236703 | 1.243563888 | 1.140090384 |
| 211634_x_at | M24669.1 | Hs.153261 | 0.0112 | 3.0444 | IGHG1 | 2.59388633 | 1.360479452 | 1.073739062 |
| 207543_s_at | NM_000917.1 | Hs.76768 | 0.0109 | 3.0555 | P4HA1 | 1.733925706 | 1.252700489 | 1.186234466 |
| 210108_at | BE550599 | Hs.399966 | 0.0109 | 3.0595 | CACNA1D | 1.489860167 | 1.384488076 | 1.495170472 |
| 203932_at | NM_002118.1 | Hs.1162 | 0.0104 | 3.0864 | HLA-DMB | 1.524664331 | 1.189013209 | 1.06592707 |
| 203915_at | NM_002416.1 | Hs.77367 | 0.0102 | 3.0926 | CXCL9 | 1.909087593 | 1.2391476 | 1.074101762 |
| 221011_s_at | NM_030915.1 | Hs.57209 | 0.0096 | 3.1259 | LBH | 1.81373734 | 1.470327604 | 1.270395433 |
| 200016_x_at | NM_002136.1 | Hs.376844 | 0.0096 | 3.1299 | HNRPA1 | 1.463719776 | 1.22408099 | 1.215486347 |
| 213187_x_at | BG538564 | Hs.433669 | 0.0093 | 3.1451 | FTL | 1.664543605 | 1.167743171 | 1.128725875 |
| 206858_s_at | NM_004503.1 | Hs.820 | 0.0093 | 3.1466 | HOXC6 | 1.855396742 | 1.814474567 | 2.200409215 |
| 208308_s_at | NM_000175.1 | Hs.406458 | 0.0091 | 3.1586 | GPI | 1.719772684 | 1.349627658 | 1.566825826 |
| 225155_at | BG339050 | Hs.292457 | 0.0088 | 3.1758 | LOC389414 | 1.699552974 | 1.495191613 | 1.42639293 |
| 200910_at | NM_005998.1 | Hs.1708 | 0.0083 | 3.21 | CCT3 | 1.636454945 | 1.407382031 | 1.738311083 |
| 201417_at | NM_003107.1 | Hs.351928 | 0.008 | 3.2293 | SOX4 | 1.970734373 | 1.650462431 | 1.909514117 |
| 200967_at | NM_000942.1 | Hs.394389 | 0.0078 | 3.2452 | PPIB | 1.662514576 | 1.1363543 | 2.158290879 |
| 201947_s_at | NM_006431.1 | Hs.432970 | 0.0078 | 3.2475 | CCT2 | 1.542573507 | 1.444834092 | 1.532058132 |
| 208638_at | BE910010 | Hs.372429 | 0.0077 | 3.2521 | ATP6V1C2 | 1.583571942 | 1.051678053 | 1.649215708 |
| 213088_s_at | BF240590 | Hs.44131 | 0.0077 | 3.2524 | DNAJC9 | 1.522969245 | 1.19041669 | 1.101924249 |
| 201892_s_at | NM_000884.1 | Hs.75432 | 0.0075 | 3.2688 | IMPDH2 | 1.545438098 | 1.476483085 | 1.73248107 |
| 200921_s_at | NM_001731.1 | Hs.77054 | 0.0069 | 3.3146 | BTG1 | 1.737055883 | 1.190188986 | 1.085456613 |
| 208650_s_at | BG327863 | Hs.375108 | 0.0067 | 3.3288 | CD24 | 1.829886814 | 1.355111901 | 1.591094884 |
| 233955_x_at | AK001782.1 | Hs.15093 | 0.0067 | 3.3325 | HSPC195 | 1.532399783 | 1.179795978 | 1.338839462 |
| 210338_s_at | AB034951.1 | Hs.180414 | 0.0066 | 3.3376 | HSPA8 | 1.68010557 | 1.41400935 | 1.538594921 |
| 229742_at | AA420989 | Hs.97896 | 0.0065 | 3.3477 | LOC145853 | 1.576219764 | 1.281197519 | 1.630748937 |
| 216207_x_at | AW408194 | Hs.390427 | 0.0063 | 3.3683 | IGKC | 2.280006856 | 1.312304195 | 0.97191288 |
| 200052_s_at | NM_004515.1 | Hs.75117 | 0.0062 | 3.3732 | ILF2 | 1.500432046 | 1.179963924 | 1.395549103 |
| 200751_s_at | BE898861 | Hs.406125 | 0.0061 | 3.3834 | HNRPC | 1.534667928 | 1.184841638 | 1.366841459 |
| 205133_s_at | NM_002157.1 | Hs.1197 | 0.006 | 3.3941 | HSPE1 | 1.563125779 | 1.432509648 | 1.587948037 |
| 202345_s_at | NM_001444.1 | Hs.153179 | 0.0059 | 3.4071 | FABP5 | 1.540717022 | 1.936910992 | 2.933164929 |
| 224997_x_at | AL575306 | Hs.352114 | 0.0057 | 3.4183 | LOC283120 | 1.850665142 | 1.121867318 | 1.03987769 |
| 226243_at | BF590958 | Hs.293943 | 0.0052 | 3.4762 | LOC391356 | 1.594266731 | 1.313820503 | 1.983449106 |
| 226711_at | BF590117 | Hs.106131 | 0.005 | 3.4963 | HTLF | 1.605953506 | 1.113911789 | 1.041441881 |
| 222976_s_at | BC000771.1 | Hs.85844 | 0.0049 | 3.508 | TPM3 | 1.595051354 | 1.196763387 | 1.15890854 |
| 225655_at | AK025578.1 | Hs.108106 | 0.0048 | 3.5199 | UHRF1 | 1.633324349 | 1.262569313 | 1.076492985 |
| 201730_s_at | BF110993 | Hs.169750 | 0.0046 | 3.5406 | TPR | 1.65067228 | 1.276077237 | 1.489979997 |
| 209301_at | M36532.1 | Hs.155097 | 0.0045 | 3.553 | CA2 | 1.775302858 | 1.022589313 | 1.018671643 |
| 217989_at | NM_016245.1 | Hs.12150 | 0.0043 | 3.578 | RETSDR2 | 1.723038343 | 1.105299082 | 1.319059719 |
| 212884_x_at | AI358867 | Hs.169401 | 0.0043 | 3.5876 | APOC4 | 2.131295433 | 1.351949253 | 1.23086617 |
| 202016_at | NM_002402.1 | Hs.79284 | 0.0041 | 3.6079 | MEST | 1.529459472 | 1.310502398 | 1.081141622 |
| 223034_s_at | BC000152.2 | Hs.355906 | 0.0041 | 3.6103 | NICE-3 | 1.66226553 | 1.326721145 | 1.506773141 |
| 229429_x_at | AA863228 | Hs.379811 | 0.0041 | 3.616 | IMAGE: 6191689 | 1.515106064 | 1.321222321 | 1.214669373 |
| 200003_s_at | NM_000991.1 | Hs.356371 | 0.0037 | 3.6632 | RPL28 | 1.550101477 | 1.355858477 | 1.452357975 |
| 213366_x_at | AV711183 | Hs.155433 | 0.0036 | 3.6807 | ATP5C1 | 1.529032497 | 1.119093162 | 1.331117036 |
| 225340_s_at | BG107845 | Hs.278672 | 0.0036 | 3.6813 | M11S1 | 1.582161146 | 1.287025159 | 1.498201492 |
| 200738_s_at | NM_000291.1 | Hs.78771 | 0.0036 | 3.6839 | PGK1 | 1.683510425 | 1.072151437 | 1.244584776 |
| 211935_at | D31885.1 | Hs.75249 | 0.0035 | 3.7007 | ARL6IP | 1.586948602 | 1.45583784 | 1.354948119 |
| 230875_s_at | AW068936 | Hs.29189 | 0.0035 | 3.7026 | ATP11A | 1.893995893 | 1.28867667 | 1.284361224 |
| 211798_x_at | AB001733.1 | Hs.102950 | 0.0032 | 3.7431 | IGLJ3 | 2.253481227 | 1.190254197 | 0.949978045 |
| 201258_at | NM_001020.1 | Hs.397609 | 0.0032 | 3.7555 | RPS16 | 1.529474743 | 1.257275471 | 1.240593812 |
| 200046_at | NM_001344.1 | Hs.82890 | 0.0031 | 3.7691 | DAD1 | 1.503927044 | 1.23704027 | 1.45535289 |
| 200023_s_at | NM_003754.1 | Hs.7811 | 0.0031 | 3.7759 | EIF3S5 | 1.492677918 | 1.053270057 | 1.303541027 |
| 200806_s_at | BE256479 | Hs.79037 | 0.003 | 3.7832 | HSPD1 | 1.963190492 | 1.71958264 | 1.754752854 |
| 201268_at | NM_002512.1 | Hs.433416 | 0.003 | 3.7882 | NME2 | 1.52341029 | 1.365069689 | 1.56867628 |
| 224598_at | BF570193 | Hs.4867 | 0.003 | 3.7948 | MGAT4B | 1.622431221 | 1.358611937 | 1.359348866 |
| 200608_s_at | NM_006265.1 | Hs.81848 | 0.0028 | 3.8326 | RAD21 | 1.60409789 | 1.30816732 | 1.284445316 |
| 213872_at | BE465032 | Hs.7779 | 0.0028 | 3.8362 | C6ORF62 | 1.646199498 | 1.17809594 | 1.200055245 |
| 218188_s_at | NM_012458.1 | Hs.23410 | 0.0027 | 3.8535 | MKNK2 | 1.503773313 | 1.349464531 | 1.566222625 |
| 204714_s_at | NM_000130.2 | Hs.30054 | 0.0026 | 3.8747 | F5 | 2.165592205 | 1.679183224 | 1.676626265 |
| 200077_s_at | D87914.1 | Hs.281960 | 0.0025 | 3.8866 | OAZ1 | 1.524134063 | 1.262277281 | 1.230514687 |
| 213864_s_at | AI985751 | Hs.302949 | 0.0025 | 3.8979 | NAP1L1 | 1.67220728 | 1.394334759 | 1.301162479 |
| 201577_at | NM_000269.1 | Hs.118638 | 0.0024 | 3.9233 | NME1 | 1.762629579 | 1.473601738 | 1.768856036 |
| 212828_at | AL157424.1 | Hs.417119 | 0.0024 | 3.9288 | SYNJ2 | 1.558960833 | 1.215873328 | 1.26061887 |
| 200074_s_at | U16738.1 | Hs.406451 | 0.0022 | 3.9762 | RPL14 | 1.554434561 | 1.307651132 | 1.627182376 |
| 202779_s_at | NM_014501.1 | Hs.174070 | 0.0022 | 3.9798 | E2-EPF | 1.567646954 | 1.295809911 | 1.146938081 |
| 211765_x_at | BC005982.1 | Hs.401787 | 0.0021 | 3.9977 | PPIA | 1.573983335 | 1.425560154 | 1.374534514 |
| 208864_s_at | AF313911.1 | Hs.432922 | 0.0019 | 4.0434 | TXN | 1.787154285 | 1.626360765 | 1.669494713 |
| 225541_at | BE274422 | Hs.380933 | 0.0019 | 4.0627 | LOC200916 | 1.542963884 | 1.631682436 | 1.778268586 |
| 212282_at | L19183.1 | Hs.199695 | 0.0019 | 4.0627 | MAC30 | 1.753247965 | 1.348307061 | 1.511823697 |
| 210024_s_at | AB017644.1 | Hs.4890 | 0.0018 | 4.0888 | UBE2E3 | 1.636706653 | 1.501673356 | 1.582518098 |
| 201923_at | NM_006406.1 | Hs.83383 | 0.0018 | 4.0895 | PRDX4 | 2.092722507 | 1.503078231 | 2.357995857 |
| 212085_at | AA916851 | Hs.397980 | 0.0018 | 4.0911 | SLC25A6 | 1.904390097 | 1.32734063 | 1.618458407 |
| 204934_s_at | NM_002151.1 | Hs.823 | 0.0018 | 4.1026 | HPN | 1.960192099 | 1.784641097 | 2.452778498 |
| 227558_at | AI570531 | Hs.5637 | 0.0017 | 4.1127 | CBX4 | 1.50757404 | 1.452066169 | 1.692781886 |
| 203663_s_at | NM_004255.1 | Hs.434076 | 0.0017 | 4.1185 | COX5A | 1.613062245 | 1.374743959 | 1.755673991 |
| 218226_s_at | NM_004547.2 | Hs.227750 | 0.0016 | 4.1453 | NDUFB4 | 1.742463447 | 1.359340208 | 1.586965718 |
| 200089_s_at | AI953886 | Hs.286 | 0.0016 | 4.1592 | RPL4 | 1.53268956 | 1.115222706 | 1.484095711 |
| 201091_s_at | BE748755 | Hs.406384 | 0.0015 | 4.1926 | CBX3 | 1.524136246 | 1.380672462 | 1.217491886 |
| 224779_s_at | AI193090 | Hs.406548 | 0.0015 | 4.2067 | FLJ22875 | 1.558101693 | 1.273161615 | 1.427956916 |
| 206052_s_at | NM_006527.1 | Hs.75257 | 0.0015 | 4.2109 | SLBP | 1.521358079 | 1.252449739 | 1.271120912 |
| 200099_s_at | AL356115 | — | 0.0015 | 4.2143 | RPS3A | 1.520554944 | 1.143653686 | 1.248258538 |
| 203593_at | NM_012120.1 | Hs.374340 | 0.0014 | 4.2363 | CD2AP | 1.602425228 | 1.242316644 | 1.5150563 |
| 223015_at | AF212241.1 | Hs.332404 | 0.0014 | 4.2391 | EIF2A | 1.497306539 | 1.242359457 | 1.344582841 |
| 219065_s_at | NM_015955.1 | Hs.20814 | 0.0013 | 4.268 | CGI-27 | 1.507583206 | 1.328804481 | 1.277143137 |
| 226431_at | AK025007.1 | Hs.283707 | 0.0013 | 4.2731 | FLJ38771 | 1.598874153 | 1.399493212 | 1.627928293 |
| 205967_at | NM_003542.2 | Hs.46423 | 0.0013 | 4.3018 | HIST1H4C | 1.555503253 | 1.087464227 | 1.116349924 |
| 212582_at | AB040884.1 | Hs.109694 | 0.0012 | 4.311 | OSBPL8 | 1.715379905 | 1.301229214 | 1.229883545 |
| 215785_s_at | AL161999.1 | Hs.258503 | 0.0012 | 4.3179 | CYFIP2 | 1.56203664 | 1.078404104 | 1.115902029 |
| 200005_at | NM_003753.1 | Hs.55682 | 0.0012 | 4.3351 | EIF3S7 | 1.486307905 | 1.092082639 | 1.35598979 |
| 201406_at | NM_021029.1 | Hs.178391 | 0.0012 | 4.3469 | RPL36AL | 1.622586596 | 1.318939119 | 1.315227712 |
| 202589_at | NM_001071.1 | Hs.29475 | 0.0011 | 4.3893 | TYMS | 1.767443638 | 1.222542727 | 1.002726592 |
| 200705_s_at | NM_001959.1 | Hs.275959 | 0.0011 | 4.4036 | EEF1B2 | 1.760982804 | 1.031697881 | 1.234933 |
| 203381_s_at | N33009 | Hs.169401 | 0.001 | 4.4505 | APOE | 3.625071725 | 1.645066079 | 1.546251347 |
| 201909_at | NM_001008.1 | Hs.180911 | 0.001 | 4.4516 | RPS4Y | 1.599654206 | 1.115641351 | 1.24634976 |
| 200651_at | NM_006098.1 | Hs.5662 | 0.0009 | 4.4929 | GNB2L1 | 1.588142549 | 1.229268774 | 1.528267857 |
| 204026_s_at | NM_007057.1 | Hs.42650 | 0.0009 | 4.4937 | ZWINT | 1.59878202 | 1.294302945 | 1.152947206 |
| 211430_s_at | M87789.1 | Hs.300697 | 0.0009 | 4.5085 | IGHG3 | 6.771934405 | 1.802655294 | 1.254577557 |
| 222981_s_at | BC000896.1 | Hs.236494 | 0.0008 | 4.5616 | RAB10 | 1.529122674 | 1.169831372 | 1.184935217 |
| 204170_s_at | NM_001827.1 | Hs.83758 | 0.0007 | 4.6462 | CKS2 | 1.505806628 | 1.351484868 | 1.316478404 |
| 202233_s_at | NM_006004.1 | Hs.73818 | 0.0006 | 4.7216 | UQCRH | 1.507080143 | 1.407548974 | 1.450326381 |
| 213941_x_at | AI970731 | Hs.301547 | 0.0006 | 4.7385 | RPS7 | 1.736561496 | 1.299553424 | 1.383761007 |
| 201931_at | NM_000126.1 | Hs.169919 | 0.0006 | 4.7667 | ETFA | 1.518847136 | 1.235640895 | 1.484000826 |
| 200062_s_at | L05095.1 | Hs.356255 | 0.0006 | 4.7681 | RPL30 | 1.477700403 | 1.325310565 | 1.28346032 |
| 200024_at | NM_001009.1 | Hs.356019 | 0.0004 | 4.9825 | RPS5 | 1.543956946 | 1.237096382 | 1.41156327 |
| 212320_at | BC001002.1 | Hs.179661 | 0.0004 | 5.0086 | OK/SW-CL.56 | 1.549636979 | 1.09363087 | 1.144937515 |
| 221253_s_at | NM_030810.1 | Hs.6101 | 0.0003 | 5.1364 | TXNDC5 | 1.673690073 | 1.214164697 | 1.547677512 |
| 203213_at | AL524035 | Hs.334562 | 0.0003 | 5.1385 | CDC2 | 1.701034927 | 1.283019117 | 1.112690554 |
| 210027_s_at | M80261.1 | Hs.73722 | 0.0003 | 5.1408 | APEX1 | 1.569470289 | 1.267708436 | 1.408847905 |
| 200657_at | NM_001152.1 | Hs.79172 | 0.0003 | 5.1983 | SLC25A5 | 1.901741191 | 1.22604677 | 1.387961859 |
| 234000_s_at | AJ271091.1 | Hs.260622 | 0.0003 | 5.2335 | HSPC121 | 1.938344455 | 1.478824195 | 1.874867572 |
| 200022_at | NM_000979.1 | Hs.405036 | 0.0003 | 5.2504 | RPL18 | 1.498415215 | 1.119390181 | 1.333477902 |
| 212298_at | BE620457 | Hs.69285 | 0.0003 | 5.256 | NRP1 | 1.957544223 | 1.040349319 | 1.015201245 |
| 224841_x_at | BF316352 | Hs.289721 | 0.0002 | 5.3063 | LOC348531 | 1.857150338 | 1.760196963 | 1.784970922 |
| 203316_s_at | NM_003094.1 | Hs.334612 | 0.0002 | 5.3428 | SNRPE | 1.806026674 | 1.369724754 | 1.387729965 |
| 214512_s_at | NM_006713.1 | Hs.349506 | 0.0002 | 5.3545 | PC4 (RNA pol II cofactor4) | 1.532818871 | 1.168971448 | 1.141341586 |
| 200025_s_at | NM_000988.1 | Hs.402678 | 0.0002 | 5.3774 | RPL27 | 1.508452832 | 1.19030353 | 1.243386992 |
| 225681_at | AA584310 | Hs.283713 | 0.0002 | 5.3796 | CTHRC1 | 2.020161016 | 1.80816774 | 0.951729083 |
| 201292_at | NM_001067.1 | Hs.156346 | 0.0002 | 5.3883 | TOP2A | 1.833549424 | 1.291691262 | 1.079088914 |
| 200029_at | NM_000981.1 | Hs.252723 | 0.0002 | 5.4248 | RPL19 | 1.521872043 | 1.194839681 | 1.312202279 |
| 219315_s_at | NM_024600.1 | Hs.25549 | 0.0002 | 5.4645 | FLJ20898 | 1.64775771 | 0.990110268 | 0.953024778 |
| 201202_at | NM_002592.1 | Hs.78996 | 0.0002 | 5.5703 | PCNA | 1.669445435 | 1.205345044 | 1.17023514 |
| 213801_x_at | AW304232 | Hs.406309 | 0.0002 | 5.6419 | LAMR1 | 1.632088937 | 1.399421585 | 1.383008918 |
| 211762_s_at | BC005978.1 | Hs.159557 | 0.0001 | 5.6456 | KPNA2 | 1.755103495 | 1.29090564 | 1.0705669 |
| 211963_s_at | AL516350 | Hs.82425 | 0.0001 | 5.6682 | ARPC5 | 1.586387629 | 1.137184649 | 1.069876176 |
| 215157_x_at | AI734929 | Hs.172182 | 0.0001 | 5.7526 | PABPC1 | 1.6139613 | 1.411844073 | 1.486133943 |
| 221923_s_at | AA191576 | Hs.355719 | 0.0001 | 5.7669 | NPM1 | 1.511565517 | 1.347070601 | 1.495278592 |
| 209773_s_at | BC001886.1 | Hs.75319 | 0.0001 | 5.8026 | RRM2 | 1.648429002 | 1.136861988 | 1.084243831 |
| 210470_x_at | BC003129.1 | Hs.172207 | 0.0001 | 5.8383 | NONO | 1.539777316 | 1.18853499 | 1.265461231 |
| 212433_x_at | AA630314 | Hs.356360 | 0.0001 | 5.8503 | RPS2 | 1.523462718 | 1.358219429 | 1.33114119 |
| 200002_at | NM_007209.1 | Hs.182825 | 0.0001 | 5.976 | RPL35 | 1.551069832 | 1.305374553 | 1.391704639 |
| 213175_s_at | AL049650 | Hs.83753 | 0.0001 | 5.9948 | SNRPB | 1.576875717 | 1.135824457 | 1.139655055 |
| 200081_s_at | BE741754 | Hs.380843 | 0 | 6.4154 | RPS6 | 1.483436564 | 1.122173181 | 1.255583773 |
| 202503_s_at | NM_014736.1 | Hs.81892 | 0 | 6.5147 | KIAA0101 | 1.790877795 | 1.270030091 | 1.192391312 |
| 218039_at | NM_016359.1 | Hs.279905 | 0 | 6.5894 | ANKT | 1.906301812 | 1.308144135 | 1.15463799 |
| 200823_x_at | NM_000992.1 | Hs.350068 | 0 | 6.6909 | RPL29 | 1.660135008 | 1.25782313 | 1.461476429 |
| 201592_at | NM_003756.1 | Hs.58189 | 0 | 6.747 | EIF3S3 | 1.624202671 | 1.284913932 | 1.214917882 |
| 200826_at | NM_004597.3 | Hs.397090 | 0 | 8.4509 | SNRPD2 | 1.668850891 | 1.095430311 | 1.237917587 |
| 224930_x_at | BE559788 | Hs.99858 | 0 | 8.519 | RPL7A | 1.569935841 | 1.312951533 | 1.518175682 |
| 203554_x_at | NM_004219.2 | Hs.252587 | 0 | 8.678 | PTTG1 | 1.598399511 | 1.224970621 | 1.036680081 |

TABLE 3
Significance of the genes validated by Taqman real time PCR. Kruskal-Wallis Test was done
to compare the medians between the groups. All seven validated down-regulated genes (PRIMA1,
TU3A, KIAA1210, FLJ14084; SVIL, SORBS1 and C21orf63) are significantly decreased in
Metastatic, Gleason 9 and Gleason 6 grades compared to benign tissues. The increase in the
expression of genes (e.g., MAL2, MLP, SOX4 and FABP5) with 4-way null hypothesis and the
2-way null hypothesis of normal vs Gleason 6 tumors was significant. Two way null hypothesis of
normal vs Metastatic was not significant for upregulated genes.
Kruskal-Wallis Test P-values

| Gene = | SORBS1 | C21orf 63 | SVIL | PRIMA1 | FLJ14084 | TU3A | KIAA1210 | SOX4 | MLP | FABP5 | MAL2 |
|---|---|---|---|---|---|---|---|---|---|---|---|
| Comparison | Down regulated | | | | | | | Up regulated | | | |
| Nrml-Met- | 0.0000 | 0.0000 | 0.0000 | 0.0000 | 0.0000 | 0.0001 | 0.0001 | 0.0012 | 0.0032 | 0.0126 | 0.0358 |
| G6-G9 | | | | | | | | | | | |
| Met-G6-G9 | 0.0002 | 0.0021 | 0.0044 | 0.0110 | 0.0099 | 0.0098 | 0.0026 | 0.1096 | 0.4945 | 0.0316 | 0.6473 |
| Nrml-Met | 0.0043 | 0.0043 | 0.0043 | 0.0043 | 0.0043 | 0.0043 | 0.0043 | 0.0918 | 0.2723 | 0.5101 | 0.0923 |
| Nrml-G6 | 0.0002 | 0.0002 | 0.0002 | 0.0004 | 0.0006 | 0.0002 | 0.0010 | 0.0061 | 0.0014 | 0.0097 | 0.0339 |
| Nrml-G9 | 0.0027 | 0.0001 | 0.0002 | 0.0003 | 0.0004 | 0.0011 | 0.0022 | 0.0002 | 0.0006 | 0.0998 | 0.0061 |
| Met-G6 | 0.0398 | 0.9580 | 0.0019 | 0.0027 | 0.0052 | 0.0037 | 0.0019 | 0.1021 | 0.6350 | 0.0268 | 0.4292 |
| Met-G9 | 0.0052 | 0.0114 | 0.0040 | 0.0145 | 0.0068 | 0.0088 | 0.0017 | 0.1898 | 0.5409 | 0.0734 | 0.8614 |
| G6-G9 | 0.0007 | 0.0021 | 0.8644 | 0.8452 | 0.8644 | 0.7884 | 0.9805 | 0.1497 | 0.2614 | 0.1243 | 0.4792 |

NOTES:
The 4-way null hypothesis is that the four medians are the same
The 3-way null hypothesis is that the three medians are the same
The 2-way null hypotheses are that the pair-wise medians are the same
Genes were sorted by the 4-way p-value

1. A method for detecting, or for detecting and distinguishing between or among prostate cell proliferative disorders or stages thereof in a subject comprising:
obtaining, from the subject, a biological sample; and
determining, using a suitable assay, the expression level of at least one gene or sequence selected from the group consisting of: ZNF185 (SEQ ID NOS:1 and 2); PSP94 (SEQ ID NOS:29 and 30); BPAG1 (SEQ ID NO:31); SORBS1 (SEQ ID NOS:32 and 33); C21orf63 (SEQ ID NO:34); SVIL (SEQ ID NOS:35 and 36); PRIMA1 (SEQ ID NO:37); FLJ14084 (SEQ ID NOS:38 and 39); TU3A (SEQ ID NOS:40 and 41); KIAA1210 (SEQ ID NO:42); SOX4 (SEQ ID NOS:43 and 44); MLP (SEQ ID NOS:45 and 46); FABP5 (SEQ ID NOS:47 and 48); MAL2 (SEQ ID NOS:49 and 50); Erg-2 (SEQ ID NOS: 51 and 52); and sequences that hybridize under high stringency thereto, whereby detecting and distinguishing between or among prostate cell proliferative disorders or stages thereof is, at least in part, afforded.

2. The method according to claim 1, wherein said expression level is determined by detecting the presence, absence or level of mRNA transcribed from said gene or sequence.

3. The method according to claim 1, wherein said expression level is determined by detecting the presence, absence or level of a polypeptide encoded by said gene or sequence.

4. The method according to claim 1, wherein detecting and distinguishing between or among prostate cell proliferative disorders or stages thereof is, at least in part, based on a decrease in expression of at least one gene or sequence selected from the group consisting of: ZNF185 (SEQ ID NOS:1 and 2); PSP94 (SEQ ID NOS:29 and 30); BPAG1 (SEQ ID NO:31); SORBS1 (SEQ ID NOS:32 and 33); C21orf63 (SEQ ID NO:34); SVIL (SEQ ID NOS:35 and 36); PRIMA1 (SEQ ID NO:37); FLJ14084 (SEQ ID NOS:38 and 39); TU3A (SEQ ID NOS:40 and 41); KIAA1210 (SEQ ID NO:42); and sequences that hybridize under high stringency thereto.

5. The method according to claim 1, wherein detecting and distinguishing between or among prostate cell proliferative disorders or stages thereof is, at least in part, based on a increase in expression of at least one gene or sequence selected from the group consisting of: SOX4 (SEQ ID NOS:43 and 44); MLP (SEQ ID NOS:45 and 46); FABP5 (SEQ ID NOS:47 and 48); MAL2 (SEQ ID NOS:49 and 50); Erg-2 (SEQ ID NOS: 51 and 52); and sequences that hybridize under high stringency thereto.

6. The method according to claim 3, wherein said polypeptide is detected by at least one method selected from the group consisting of immunoassay, ELISA immunoassay, radioimmunoassay, and antibody.

7. The method according to claim 1 wherein said expression is determined by detecting the presence or absence of CpG methylation within said gene or sequence, wherein hypermethylation indicates the presence of, or stage of the prostate cell proliferative disorder.

8. The method according to claim 7, wherein expression is of at least one gene or sequence selected from the group consisting of: ZNF185 (SEQ ID NOS:1 and 2); SVIL (SEQ ID NOS:35 and 36); PRIMA1 (SEQ ID NO:37); FLJ14084 (SEQ ID NOS:38 and 39); TU3A (SEQ ID NOS:40 and 41); KIAA1210 (SEQ ID NO:42); and sequences that hybridize under high stringency thereto.

9. A method for detecting, or for detecting and distinguishing between or among prostate cell proliferative disorders or stages thereof in a subject, comprising:
obtaining, from the subject, a biological sample having genomic DNA; and
contacting genomic DNA obtained from the subject with at least one reagent, or series of reagents that distinguishes between methylated and non-methylated CpG dinucleotides within at least one target region of the genomic DNA, wherein the target region comprises, or hybridizes under stringent conditions to at least 16 contiguous nucleotides of at least one sequence selected from the group consisting of SEQ ID NOS:1, 29, 31, 32, 34, 35, 37, 38, 40, 42, 43, 45, 47, 49, 51, and complements thereof, wherein said contiguous nucleotides comprise at least one CpG dinucleotide sequence, and whereby detecting, or detecting and distinguishing between or among colon cell proliferative disorders or stages thereof is, at least in part, afforded.

10. The method of claim 9, wherein normal, non-prostate cell proliferative disorders, or adjacent benign tissues are distinguished from at least one condition selected from the group consisting of: intermediate, T2, Gleason score 6 lymph node positive and negative; high grade,T3, Gleason score 9 lymph node positive and negative; prostatic adenocarcinoma; and metastatic tumors.

11. The method of claim 9, wherein adjacent benign tissue is distinguished from at least one condition selected from the group consisting of: intermediate, T2, Gleason score 6 lymph node positive and negative; high grade,T3, Gleason score 9 lymph node positive and negative; prostatic adenocarcinoma; and metastatic tumors.

12. The method of claim 9, wherein adjacent benign tissue is distinguished from at least one condition selected from the group consisting of: intermediate, T2, Gleason score 6 lymph node positive and negative; high grade,T3, Gleason score 9 lymph node positive and negative; prostatic adenocarcinoma; and metastatic tumors, and wherein the target region comprises, or hybridizes under stringent conditions to at least 16 contiguous nucleotides of a sequence selected from the group consisting of ZNF185 (SEQ ID NO:1); PSP94 (SEQ ID NO:29); BPAG1 (SEQ ID NO:31); SORBS1 (SEQ ID NO:32); C21orf63 (SEQ ID NO:34); SVIL (SEQ ID NS:35); PRIMA1 (SEQ ID NO:37); FLJ14084 (SEQ ID NO:38); TU3A (SEQ ID NO:40); KIAA1210 (SEQ ID NO:42); and sequences complementary thereto.

13. The method of claim 12, wherein adjacent benign tissue is distinguished from at least one condition selected from the group consisting of: intermediate, T2, Gleason score 6 lymph node positive and negative; high grade,T3, Gleason score 9 lymph node positive and negative; prostatic adenocarcinoma; and metastatic tumors, and wherein the target region comprises, or hybridizes under stringent conditions to at least 16 contiguous nucleotides of a sequence selected from the group consisting of ZNF185 (SEQ ID NO:1); SVIL (SEQ ID NO:35); PRIMA1 (SEQ ID NO:37); FLJ14084 (SEQ ID NO:38); TU3A (SEQ ID NO:40); KIAA1210 (SEQ ID NO:42); and sequences complementary thereto.

14. The method of claim 9, wherein tissues originating from the prostate are distinguished from tissues of non-prostate origin.

15. The method of claim 9, wherein prostate cell proliferative disorders are distinguished from healthy tissues, and wherein the target region comprises, or hybridizes under stringent conditions to at least 16 contiguous nucleotides of a sequence selected from the group consisting of ZNF185 (SEQ ID NO:1); PSP94 (SEQ ID NO:29); BPAG1 (SEQ ID NO:31); SORBS1 (SEQ ID NO:32); C21orf63 (SEQ ID NO:34); SVIL (SEQ ID NO:35); PRIMA1 (SEQ ID NO:37); FLJ14084 (SEQ ID NO:38); TU3A (SEQ ID NO:40); KIAA1210 (SEQ ID NO:42); and sequences complementary thereto.

16. A method for detecting, or for detecting and distinguishing between or among prostate cell proliferative disorders or stages thereof in a subject, comprising:
obtaining, from a subject, a biological sample having genomic DNA;
contacting the genomic DNA, or a fragment thereof, with one reagent or a plurality of reagents that distinguishes between methylated and non methylated CpG dinucleotide sequences within at least one target sequence of the genomic DNA, or fragment thereof, wherein the target sequence comprises, or hybridizes under stringent conditions to, at least 16 contiguous nucleotides of a sequence taken from the group consisting of SEQ ID NOS:1, 29, 31, 32, 34, 35, 37, 38, 40, 42, 43, 45, 47, 49, 51, and complements thereof, said contiguous nucleotides comprising at least one CpG dinucleotide sequence; and
determining, based at least in part on said distinguishing, the methylation state of at least one target CpG dinucleotide sequence, or an average, or a value reflecting an average methylation state of a plurality of target CpG dinucleotide sequences, whereby detecting, or detecting and distinguishing between or among prostate cell proliferative disorders or stages thereof is, at least in part, afforded.

17. The method of claim 16, wherein detecting, or detecting and distinguishing between or among prostate cell proliferative disorders or stages thereof comprises detecting, or detecting and distinguishing between or among one or more tissues selected from the group consisting of: adjacent benign tissues; intermediate, T2, Gleason score 6 lymph node positive or negative tissue; high grade, T3, Gleason score 9 lymph node positive or negative tissue; prostatic adenocarcinoma; and metastatic tumors.

18. The method of claim 16, wherein distinguishing between methylated and non methylated CpG dinucleotide sequences within the target sequence comprises converting unmethylated cytosine bases within the target sequence to uracil or to another base that is detectably dissimilar to cytosine in terms of hybridization properties.

19. The method of claim 16, wherein distinguishing between methylated and non methylated CpG dinucleotide sequences within the target sequence(s) comprises methylation state-dependent conversion or non-conversion of at least one CpG dinucleotide sequence to the corresponding converted or non-converted dinucleotide sequence.

20. The method of claim 16, wherein the biological sample is selected from the group consisting of cell lines, histological slides, biopsies, paraffin-embedded tissue, bodily fluids, ejaculate, urine, blood, and combinations thereof.

21. The method of claim 16, wherein distinguishing between methylated and non methylated CpG dinucleotide sequences within the target sequence comprises use of at least one nucleic acid molecule or peptide nucleic acid (PNA) molecule comprising, in each case a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof.

22. The method of claim 21, wherein the contiguous sequence comprises at least one CpG, TpG or CpA dinucleotide sequence.

23. The method of claim 21, comprising use of at least two such nucleic acid molecules, or peptide nucleic acid (PNA) molecules.

24. The method of claim 21, comprising use of at least two such nucleic acid molecules as primer oligonucleotides for the amplification of a bisulfite-converted sequence derived from a sequence selected from the group consisting of SEQ ID NOS:1, 29, 31, 32, 34, 35, 37, 38, 40, 42, 43, 45, 47, 49, 51; sequences that hybridize under stringent conditions therto; and complements thereof.

25. The method of claim 21, comprising use of at least four such nucleic acid molecules, peptide nucleic acid (PNA) molecules.

26. A method for detecting, or detecting and distinguishing between or among prostate cell proliferative disorders or stages thereof in a subject, comprising:
obtaining, from a subject, a biological sample having genomic DNA;
extracting or otherwise isolating the genomic DNA;
treating the genomic DNA, or a fragment thereof, with one or more reagents to convert cytosine bases that are unmethylated in the 5-position thereof to uracil or to another base that is detectably dissimilar to cytosine in terms of hybridization properties;
contacting the treated genomic DNA, or the treated fragment thereof, with an amplification enzyme and at least two primers comprising, in each case a contiguous sequence of at least 9 nucleotides that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, wherein the treated genomic DNA or the fragment thereof is either amplified to produce at least one amplificate, or is not amplified; and
determining, based on a presence or absence of, or on a property of said amplificate, the methylation state of at least one CpG dinucleotide of a sequence selected from the group consisting of SEQ ID NOS:1, 29, 31, 32, 34, 35, 37, 38, 40, 42, 43, 45, 47, 49, 51, and complements thereof, or an average, or a value reflecting an average methylation state of a plurality of said CpG dinucleotides, whereby at least one of detecting, and detecting and distinguishing between prostate cell proliferative disorders or stages thereeof is, at least in part, afforded.

27. The method of claim 26, wherein treating the genomic DNA, or the fragment thereof comprises use of a reagent selected from the group consisting of bisulfite, hydrogen sulfite, disulfite, and combinations thereof.

28. The method of claim 26, wherein contacting or amplifying comprises use of at least one method selected from the group consisting of: use of a heat-resistant DNA polymerase as the amplification enzyme; use of a polymerase lacking 5′-3′ exonuclease activity; use of a polymerase chain reaction (PCR); generation of a amplificate nucleic acid molecule carrying a detectable labels; and combinations thereof.

29. The method of claim 28, wherein the detectable amplificate label is selected from the label group consisting of: fluorescent labels; radionuclides or radiolabels; amplificate mass labels detectable in a mass spectrometer; detachable amplificate fragment mass labels detectable in a mass spectrometer; amplificate, and detachable amplificate fragment mass labels having a single-positive or single-negative net charge detectable in a mass spectrometer; and combinations thereof.

30. The method of claim 26, wherein the biological sample obtained from the subject is selected from the group consisting of cell lines, histological slides, biopsies, paraffin-embedded tissue, bodily fluids, ejaculate, urine, blood, and combinations thereof.

31. The method of claim 26, wherein detecting, or detecting and distinguishing between or among prostate cell proliferative disorders or stages thereof comprises detecting, or detecting and distinguishing between or among one or more tissues selected from the group consisting of: adjacent benign tissues; intermediate, T2, Gleason score 6 lymph node positive or negative tissue; high grade, T3, Gleason score 9 lymph node positive or negative tissue; prostatic adenocarcinoma; and metastatic tumors.

32. The method of claim 26, further comprising for the step of contacting the treated genomic DNA, the use of at least one nucleic acid molecule or peptide nucleic acid molecule comprising in each case a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, wherein said nucleic acid molecule or peptide nucleic acid molecule suppresses amplification of the nucleic acid to which it is hybridized.

33. The method of claim 32, wherein said nucleic acid molecule or peptide nucleic acid molecule is in each case modified at the 5′-end thereof to preclude degradation by an enzyme having 5′-3′ exonuclease activity.

34. The method of claim 32, wherein said nucleic acid molecule or peptide nucleic acid molecule is in each case lacking a 3′ hydroxyl group.

35. The method of claim 32, wherein the amplification enzyme is a polymerase lacking 5′-3′ exonuclease activity.

36. The method of claim 26, wherein determining comprises hybridization of at least one nucleic acid molecule or peptide nucleic acid molecule in each case comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof.

37. The method of claim 36, wherein at least one such hybridizing nucleic acid molecule or peptide nucleic acid molecule is bound to a solid phase.

38. The method of claim 36, wherein a plurality of such hybridizing nucleic acid molecules or peptide nucleic acid molecules are bound to a solid phase in the form of a nucleic acid or peptide nucleic acid array selected from the array group consisting of linear or substantially so, hexagonal or substantially so, rectangular or substantially so, and combinations thereof.

39. The method of claim 36, further comprising extending at least one such hybridized nucleic acid molecule by at least one nucleotide base.

40. The method of claim 26, wherein determining comprises sequencing of the amplificate.

41. The method of claim 26, wherein contacting or amplifying comprises use of methylation-specific primers.

42. The method of claim 26, comprising, for the contacting step, using primer oligonucleotides comprising one or more CpG; TpG or CpA dinucleotides; and further comprising, for the determining step, the use of at least one method selected from the group consisting of: hybridizing in at least one nucleic acid molecule or peptide nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof; hybridizing at least one nucleic acid molecule that is bound to a solid phase and comprises a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, hybridizing at least one nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, and extending at least one such hybridized nucleic acid molecule by at least one nucleotide base; and sequencing, in the determining step, of the amplificate.

43. The method of claim 26 comprising, for the contacting step, use of at least one nucleic acid molecule or peptide nucleic acid molecule comprising in each case a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, wherein said nucleic acid molecule or peptide nucleic acid molecule suppresses amplification of the nucleic acid to which it is hybridized; and further comprising, in the determining step, the use of at least one method selected from the group consisting of: hybridizing in at least one nucleic acid molecule or peptide nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof; hybridizing at least one nucleic acid molecule that is bound to a solid phase and comprises a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, hybridizing at least one nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, and extending at least one such hybridized nucleic acid molecule by at least one nucleotide base; and sequencing, in the determining step, of the amplificate.

44. The method of claim 26, comprising, in the contacting step, amplification by primer oligonucleotides comprising one or more CpG; TpG or CpA dinucleotides and further comprising, in the determining step, hybridizing at least one detectably labeled nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38,40, 42, 43, 45, 47, 49, 51, and complements thereof.

45. The method of claim 26, comprising, in the contacting step, the use of at least one nucleic acid molecule or peptide nucleic acid molecule comprising in each case a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof, wherein said nucleic acid molecule or peptide nucleic acid molecule suppresses amplification of the nucleic acid to which it is hybridized, and further comprising, in the determining step, hybridizing at least one detectably labeled nucleic acid molecule comprising a contiguous sequence at least 9 nucleotides in length that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof.

46. A method for detecting, or for detecting and distinguishing between or among prostate cell proliferative disorders or stages thereof in a subject, comprising:
obtaining, from a subject, a biological sample having genomic DNA;
extracting, or otherwise isolating the genomic DNA;
contacting the genomic DNA, or a fragment thereof, comprising at least 16 contiguous nucleotides of a sequence selected from the group consisting of SEQ ID NOS:1, 29, 31, 32, 34, 35, 37, 38, 40, 42, 43, 45, 47, 49, 51, complements thereof; and sequences that hybridize under stringent conditions thereto, with one or more methylation-sensitive restriction enzymes, wherein the genomic DNA is, with respect to each cleavage recognition motif thereof, either cleaved thereby to produce cleavage fragments, or not cleaved thereby; and
determining, based on a presence or absence of, or on property of at least one such cleavage fragment, the methylation state of at least one CpG dinucleotide of a sequence selected from the group consisting of SEQ ID NOS:1, 29, 31, 32, 34, 35, 37, 38, 40, 42, 43, 45, 47, 49, 51; and complements thereof, or an average, or a value reflecting an average methylation state of a plurality of said CpG dinucleotides, whereby at least one of detecting, or of detecting and differentiating between or among prostate cell proliferative disorders or stages thereof is, at least in part, afforded.

47. The method of claim 46, further comprising, prior to determining, amplifying of the digested or undigested genomic DNA.

48. The method of claim 47, wherein amplifying comprises use of at least one method selected from the group consisting of: use of a heat resistant DNA polymerase as an amplification enzyme; use of a polymerase lacking 5′-3′ exonuclease activity; use of a polymerase chain reaction (PCR); generation of a amplificate nucleic acid carrying a detectable label; and combinations thereof.

49. The method of claim 48, wherein the detectable amplificate label is selected from the label group consisting of: fluorescent labels; radionuclides or radiolabels; amplificate mass labels detectable in a mass spectrometer; detachable amplificate fragment mass labels detectable in a mass spectrometer; amplificate, and detachable amplificate fragment mass labels having a single-positive or single-negative net charge detectable in a mass spectrometer; and combinations thereof.

50. The method of claim 46, wherein the biological sample obtained from the subject is selected from the group consisting of cell lines, histological slides, biopsies, paraffin-embedded tissue, bodily fluids, ejaculate, urine, blood, and combinations thereof.

51. An isolated treated nucleic acid derived from SEQ ID NOS:1, 29, 31, 32, 34, 35, 37, 38, 40, 42, 43, 45, 47, 49, 51, and complements thereof, wherein the treatment is suitable to convert at least one unmethylated cytosine base of the genomic DNA sequence to uracil or another base that is detectably dissimilar to cytosine in terms of hybridization.

52. A nucleic acid, comprising at least 16 contiguous nucleotides of a treated genomic DNA sequence derived from a sequence selected from the group consisting of SEQ ID NOS:1, 29, 31, 32, 34, 35, 37, 38, 40, 42, 43, 45, 47, 49, 51, and complements thereof, wherein the treatment is suitable to convert at least one unmethylated cytosine base of the genomic DNA sequence to uracil or another base that is detectably dissimilar to cytosine in terms of hybridization.

53. The nucleic acid of claims 52, wherein the contiguous base sequence comprises at least one CpG, TpG or CpA dinucleotide sequence.

54. The nucleic acid of any one of claims 52 and 53, wherein the treatment comprises use of a reagent selected from the group consisting of bisulfite, hydrogen sulfite, disulfite, and combinations thereof.

55. An oligomer, comprising a sequence of at least 9 contiguous nucleotides that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof.

56. The oligomer of claim 55, comprising at least one CpG , CpA or TpG dinucleotide sequence.

57. A set of oligomers, comprising at least two oligonucleotides according, in each case, to any one of claims 55 or 56.

58. (canceled)

59. (canceled)

60. (canceled)

61. (canceled)

62. (canceled)

63. A method for manufacturing a nucleic acid array, comprising at least one of attachment of an oligomer according to any one of claims 55 or 56, or attachment of a set of oligomers or nucleic acids according to claim 57, to a solid phase.

64. An oligomer array manufactured according to claim 79.

65. The oligomer array of claim 64, wherein the oligomers are bound to a planar solid phase in the form of a lattice selected from the group consisting of linear or substantially linear lattice, hexagonal or substantially hexagonal lattice, rectangular or substantially rectangular lattice, and lattice combinations thereof.

66. (canceled)

67. The array of claim 64, wherein the solid phase surface comprises a material selected from the group consisting of silicon, glass, polystyrene, aluminum, steel, iron, copper, nickel, silver, gold, and combinations thereof.

68. A kit useful for detecting, or for detecting distinguishing between or among prostate cell proliferative disorders or stages thereof of a subject, comprising: at least one of a bisulfite reagent, and a methylation-sensitive restriction enzyme; and at least one nucleic acid molecule or peptide nucleic acid molecule comprising, in each case a contiguous sequence at least 9 nucleotides that is complementary to, or hybridizes under stringent conditions to a bisulfite-converted sequence derived from a sequence selected from the group consisting of SEQ ID NOS:1, 29, 31, 32, 34, 35, 37, 38, 40, 42, 43, 45, 47, 49, 51, and complements thereof.

69. The kit of claim 68, further comprising standard reagents for performing a methylation assay selected from the group consisting of MS-SNuPE, MSP, MethyLight, HeavyMethyl, COBRA, nucleic acid sequencing, and combinations thereof.

70. The method of any one of claims 9, 16, 26 or 46, comprising use of the kit according to claim 68.

71. (canceled)


SEQUENCE LISTING (52 sequences)

SEQ ID NO: 1 - DNA, 3614 bp, Homo sapiens
  CDS (41)..(1399)
gctacaagatgaccactgaggattacaagaagctgtgagtatgcaacgccaggcaccctacaatatcaggcgcagctctacatcaggggacaccgaggaggaggaggaggaggaggtggtgccattctcctcagatgaacagaaacggaggtcagaggctgcaagcggtgttctgaggaggacagctccccgggagcactcctacgtcctgtcagcggccaagaagagcactggcagtcctacccaggagacacaggcaccgtttatcgcgaagagggtggaggtggtggaagaggacgggccttctgagaagagccaggacccacctgctctggcaagatccactcctggctcaaacagctcaagaggtgaggaaattgtccgcctgcagatcctgacacccagggcaggactccgcctggtggccccagacgtggaaggcatgagctccagtgccacttcagtctctgctgtccctgctgataggaagagcaacagcacagcagcccaggaggatgcaaaggcagacccaaagggggccttggctgattgtgaggggaaggatgtacccaccagggtcggagaggcctggcaggagaggcctggagctccaagaggtggccaaggagacccagctgtacccgctcagcaacctgcagatcccagcaccccagagcggcagagcagccccagcggatctgagcaacttgtcagacgagagagttgtggcagtagcgtgttgactgattttgaggggaaggatgtggccaccaaggtcggagaggcctggcaggacaggcctagagccccaagaggtggccaaggagacccagctgtacccactcagcaacctgcagatcccagtaccccagaacagcagaacagccccagcggatctgagcaattcgtcagacgagagagctgcaccagcagggtgaggagcccctcgagctgcatggtcactgttactgtcactgccacatctgagcagcctcacatttatattccagcccccgcaagtgaattggactccagctctaccaccaaagggattctcttcgtgaaggagtacgtgaatgctagtgaagtgtcttctgggaagccagtatctgcacgctatagcaacgtcagcagcattgaggactcattcgccatggagaagaagcctccatgtggcagcactccatactctgagaggacaactggagggatctgtacttactgcaaccgtgagatccgagactgtccaaagattaccctagaacatcttggtatctgctgccatgaatattgctttaagtgtgggatttgcagtaaaccgatgggcgatctcctggatcagatcttcattcaccgtgacaccattcactgtgggaaatgctatgagaagctcttctagcgaccccccaccgccaggctgatcagaagctgatgactcgtggacaaatttggctgtccccagttttgccccaagttgctgtctccccttccctcacctcctccctccctgtttgatttcttcatgcttttgcccttctcaagttgaagttgcatacatccaatatcgtatcttaatgatgctatgataattgcttgtgtgtgtagcttcttgtagcttagaaagcgctttatgcccatgatgtcatttcaggctcaaccaaagaggatcaaacaggaattccatcttggcttccctaagacagattggctttctaatgagtttaagtgggcagaagtgtagggttcagtgtgtcctgactcccttgaggcttataatgggccaagttgaagactgttgatgatccctggtgggtaaattgcagacatcaaatgctagggattggcataggctagtgtttagcttgtctatttgccatatctatttttttaaatttccatacacttgtaaaagtagttagttgcttttgattgagttatatagcagtttttcatttggtcttccactcaccgttcactatatttgagtgttcccttacaggtatgttggcatgtgttggaaaatttacacaattaggtttaaattcagtaggatgtgattttgggggtggactgatcaaagtgatatctgtgtctgttggaatcttgatagctgattaatttgccctcaattctgctccctgaacttcacacataaatcttcccaagtgggttttagggtgtatagatcccagcaggattaaggaagtggaaaagcagctaacatttcttgaggctctaccacatagcaggcactgtcacagagtaatggcattaatccccataataatcctgtgaaggtgatattctcatcccatcttagacatgaggatattggaactcagagaggtggctattgcattgcgcagaacgctacagagcccatgctcttcccagagcagcacccacaaaagcaagcattgattttgtgctcagtgtgtgccaagcactgtgcagagggtacacagttcctgccaggttaacaccctcccttcaggcctcccaaaggcataggcttgcaaagagcagaaggtgtgaaatcacactcttcctctgggcatcctggatccctgaattatccccccccccatgaagtacttcaagggccaagctgcccctttccctcctctccgcccatgaaaatgcctccaaactgagatgctttcagctgagaacagatttgactcacagacattaccaaagaggagcttgtgaatccaggaaaagctccagggggctagctgatctgagcagagagctttcagtgacccattttcctgtctagactctgccttaagctagtggcaactgctggggccccaggtacttgggacatggaaactcgttggatggctgggcagatgtaagcctgtccatgcagtcagccgatcctctgctcaggttcagctggactctgccatctgtgggcccagcatcactctgtaagttccttgaaaggaagaacaaccttagagtatttctgatacaaaatgagggcctctgctcttgatttaattataaaatgtctacgtctttctccagtttctgagccctatgcacattggcttgtgggcttgttcttcctgccaaatgatcagagagggaacattccatttatttgtagtggatttcctctggagggcatgtacccacactaaataccaactgctcttcctcagctgtagtccccaacatcagacttggcacgtggtggacactaacacacaggcactcaatgaatgagtgaaggaaataaaagtcaccccccgttggtgagaaggtgcctatccccctgagtcctcagtgcaggaccagtggatgaaaggcaaggtaaagaggcccaagataggctggcttcccccgttcaaggtatagtctgcctttaagggagttttagaaccaacatgcaagacattgaaagaaatcttgcaagagccattattgacttagatccaaaacagcctctctcatgtctaaaaaggcacagaattttgcagatctgaggaagagggatgcattacctttttgcttcttttcaattgcttagtgtttctaatcatacttaatccacactaatgtgcgcaattataataaatgctaaaatatcaaaaaaaaaa

SEQ ID NO: 2 - protein, 452 aa, Homo sapiens
MQRQAPYNIRRSSTSGDTEEEEEEEVVPFSSDEQKRRSEAASGVLRRTAPREHSYVLSAAKKSTGSPTQETQAPFIAKRVEVVEEDGPSEKSQDPPALARSTPGSNSSRGEEIVRLQILTPRAGLRLVAPDVEGMSSSATSVSAVPADRKSNSTAAQEDAKADPKGALADCEGKDVPTRVGEAWQERPGAPRGGQGDPAVPAQQPADPSTPERQSSPSGSEQLVRRESCGSSVLTDFEGKDVATKVGEAWQDRPRAPRGGQGDPAVPTQQPADPSTPEQQNSPSGSEQFVRRESCTSRVRSPSSCMVTVTVTATSEQPHIYIPAPASELDSSSTTKGILFVKEYVNASEVSSGKPVSARYSNVSSIEDSFAMEKKPPCGSTPYSERTTGGICTYCNREIRDCPKITLEHLGICCHEYCFKCGICSKPMGDLLDQIFIHRDTIHCGKCYEKLF

SEQ ID NO: 3 - DNA, 23 bp, artificial sequence
  ZNF 185 forward primer
tggatgaaaggcaaggtaaagag

SEQ ID NO: 4 - DNA, 26 bp, artificial sequence
  ZNF 185 reverse primer
ttctaaaactcccttaaaggcagact

SEQ ID NO: 5 - DNA, 23 bp, artificial sequence
  ZNF 185 probe
ccaagataggctggcttcccccg

SEQ ID NO: 6 - DNA, 30 bp, artificial sequence
  PSP94 forward primer
agtgaatggataatctagtgtgcttctagt

SEQ ID NO: 7 - DNA, 25 bp, artificial sequence
  PSP94 reverse primer
gcatggctacacaatcattgactat

SEQ ID NO: 8 - DNA, 23 bp, artificial sequence
  PSP94 probe
cccaggccaggcctcattctcct

SEQ ID NO: 9 - DNA, 20 bp, artificial sequence
  BPAG1 forward primer
tcgctgaaagagcacgtcat

SEQ ID NO: 10 - DNA, 24 bp, artificial sequence
  BPAG1 reverse primer
agcaatctaaaacactgcagcttg

SEQ ID NO: 11 - DNA, 36 bp, artificial sequence
  BPAG1 probe
aatcaaagagaaagatataaattcgttcccacagcc

SEQ ID NO: 12 - DNA, 20 bp, artificial sequence
  Erg-2 forward primer
tcctgtcggacagctccaac

SEQ ID NO: 13 - DNA, 18 bp, artificial sequence
  Erg-2 reverse primer
cgggatccgtcatcttga

SEQ ID NO: 14 - DNA, 24 bp, artificial sequence
  Erg-2 probe
tgcatcacctgggaaggcaccaac

SEQ ID NO: 15 - DNA, 21 bp, artificial sequence
  1W forward primer
gcgcagttccgggtgtctgtc

SEQ ID NO: 16 - DNA, 20 bp, artificial sequence
  1W reverse primer
gcggggaggaccagcgttag

SEQ ID NO: 17 - DNA, 19 bp, artificial sequence
  1M forward primer
gcgtagtttcgggtgtttg

SEQ ID NO: 18 - DNA, 23 bp, artificial sequence
  1M reverse primer
acgaaaaaaaccaacgttaacta

SEQ ID NO: 19 - DNA, 24 bp, artificial sequence
  1U forward primer
gtgtagttttgggtgtttgttagg

SEQ ID NO: 20 - DNA, 27 bp, artificial sequence
  1U reverse primer
caaaaaaaaccaacattaactattctc

SEQ ID NO: 21 - DNA, 21 bp, artificial sequence
  2W forward primer
cctgggactccgtcagactgg

SEQ ID NO: 22 - DNA, 20 bp, artificial sequence
  2W reverse primer
gacagacacccggaactgcg

SEQ ID NO: 23 - DNA, 20 bp, artificial sequence
  2M forward primer
ttgggatttcgttagattgg

SEQ ID NO: 24 - DNA, 20 bp, artificial sequence
  2M reverse primer
aacaaacacccgaaactacg

SEQ ID NO: 25 - DNA, 24 bp, artificial sequence
  2U forward primer
tgggattttgttagattggaaagg

SEQ ID NO: 26 - DNA, 25 bp, artificial sequence
  2U reverse primer
ctaacaaacacccaaaactacacca

SEQ ID NO: 27 - DNA, 21 bp, artificial sequence
  FABP5 forward primer
ggagtgggatgggaaggaaag

SEQ ID NO: 28 - DNA, 25 bp, artificial sequence
  FABP5 reverse primer
cactccaccactaatttcccatctt

SEQ ID NO: 29 - DNA, 572 bp, homo sapiens
  CDS (33)..(377)
gtacctgtctataaggagtcctgcttatcacaatgaatgttctcctgggcagcgttgtgatctttgccaccttcgtgactttatgcaatgcatcatgctatttcatacctaatgagggagttccaggagattcaaccaggaaatgcatggatctcaaaggaaacaaacacccaataaactcggagtggcagactgacaactgtgagacatgcacttgctacgaaacagaaatttcatgttgcacccttgtttctacacctgtgggttatgacaaagacaactgccaaagaatcttcaagaaggaggactgcaagtatatcgtggtggagaagaaggacccaaaaaagacctgttctgtcagtgaatggataatctaatgtgcttctagtaggcacagggctcccaggccaggcctcattctcctctggcctctaatagtcaatgattgtgtagccatgcctatcagtaaaaagatttttgagcaaacacttgaatatgtgtgtccttttaatttataatttatgtatgcattgatttaatacacattataaagaatacaaaacatttaaaaa

SEQ ID NO: 30 - protein, 114 aa, homo sapiens
MNVLLGSVVIFATFVTLCNASCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 31 - DNA, 8930 bp, homo sapiens
gaattccggaaagaaagaacatcgtttcaggaataaaaatgcacagtagtagttatagttaccgtagcagtgattctgtgtttagtaacactaccagcactcgaaccagtcttgattcaaatgaaaatcttctcttggttcattgtggtccaacactgatcaactcttgcattagcttcggcagtgaatcctttgatggacacaggttagaaatgttgcaacagattgccaacagagttcagagggacagtgtcatctgtgaagacaaactgattcttgctggaaatgctcttcagtctgattctaaaagattagaatcaggagtgcagtttcagaatgaagcagaaattgctgggtatatacttgaatgtgagaaccttttacgccagcatgtaattgatgtacagattcttattgatggaaaatactaccaggcagatcaattggtacagagggttgcaaaactgcgtgacgaaattatggccttaaggaacgaatgttcttctgtgtacagcaaaggacgcatactgacaacagaacagacaaagctcatgatatcaggaatcactcaaagtttaaactcaggatttgcacagaccttacaccctagtctgacctcagggctgacccagagtttaacaccttccctaacctcttctagtatgacttctggcctgtcatcagggatgacttcccgcctgactccatctgtcactccagcttatacacctggtttcccatcaggattagttccaaatttcagttcaggagtagagccaaattcattgcaaactttgaagttgatgcagatccgaaaaccccttctaaagtcttctttgctggatcaaaatttaacagaagaagaaatcaatatgaaatttgttcaggatcttttgaattgggttgatgagatgcaggtacaactggaccgcactgagtggggctcagatttgccaagtgttgaaagccatttagaaaatcataaaaatgttcatagagctattgaagaatttgaatctagtctcaaagaagctaaaatcagtgagattcaaatgacagcacctcttaaactgacttatgcagaaaagttgcacagattagagagtcagtatgcaaaactcttgaatacatccaggaatcaagaacggcaccttgatacactccataattttgtaagtcgtgcgactaatgaacttatttggttgaatgaaaaagaagaggaggaagttgcttatgactggagtgagagaaacaccaacatagctaggaaaaaagattatcatgctgaattaatgagagaacttgatcaaaaggaagaaaatattaaatcagttcaggagatagcagagcagctacttctagaaaatcatccagcccggttaactattgaggcctacagagcggcaatgcagacgcagtggagctggatcttacagctctgccagtgtgtggagcagcacataaaggagaacacagcgtatttcgagtttttcaatgatgccaaagaagctactgattacttaaggaatctaaaagatgccattcagcggaagtacagctgtgatagatcaagcagcattcacaagctagaagaccttgttcaggaatcaatggaagagaaagaagaacttctgcagtacaaaagcactatagcaaacctaatgggaaaagcaaaaacaataattcaactgaagccaaggaattctgactgtccactcaaaacttctattccgatcaaagctatctgtgactacagacaaattgagataaccatttacaaagacgatgaatgtgttttggcaaataactctcatcgtgctaaatggaaggtcattagtcctactgggaatgaggctatggtcccatctgtgtgcttcaccgttcctccaccaaacaaagaagcggtggaccttgccaacagaattgagcaacagtatcagaatgtcctgactctttggcatgagtctcacataaacatgaagagtgtagtatcctggcattatctcatcaatgaaattgatagaattcgagctagcaatgtggcttcaataaagacaatgctacctggtgaacatcagcaagttctaagtaatctacaatctcgttttgaagattttctggaagatagccaggaatcccaagtcttttcaggctcagatataacacaactggaaaaggaggttaatgtatgtaagcagtattatcaagaacttcttaaatctgcagaaagagaggagcaagaggaatcagtttataatctctacatctctgaagttcgaaacattagacttcggttagagaactgtgaagatcggctgattagacagattcgaactcccctggaaagagatgatttgcatgaaagtgtgttcagaatcacagaacaggagaaactaaagaaagagctggaacgacttaaagatgatttgggaacaatcacaaataagtgtgaggagtttttcagtcaagcagcagcctcttcatcagtccctaccctacgatcagagcttaatgtggtccttcagaacatgaaccaagtctattctatgtcttccacttacatagataagttgaaaactgttaacttggggttaaaaaacactcaagctgcagaagccctcgtaaaactctatgaaactaaactgtgtgaagaagaagcagttatagctgacaagaataatattgagaatctaataagtactttaaagcaatggagatctgaagtagatgaaaagagacaggtattccatgccttagaggatgagttgcagaaagctaaagccatcagtgatgaaatgtttaaaacgtataaagaacgggaccttgattttgactggcacaaagaaaaagcagatcaattagttgaaaggtggcaaaatgttcatgtgcagattgacaacaggttacgggacttagagggcattggcaaatcactgaagtactacagagacacttaccatcctttagatgattggatccagcaggttgaaactactcagagaaagattcaggaaaatcagcctgaaaatagtaaaaccctagccacacagttgaatcaacagaagatgctggtgtccgaaatagaaatgaaacagagcaaaatggacgagtgtcaaaaatatgcagaacagtactcagctacagtgaaggactatgaattacaaacaatgacctaccgggccatggtagattcacaacaaaaatctccagtgaaacgccgaagaatgcagagttcagcagatctcattattcaagagttcatggacctaaggactcgatatactgccctggtcactctcatgacacaatatattaaatttgctggtgattcattgaagaggctggaagaggaggagattaaaaggtgtaaggagacttctgaacatggggcatattcagatctgcttcagcgtcagaaggcaacagtgcttgagaatagcaaacttacaggaaagataagtgagttggaaagaatggtagctgaactaaagaaacaaaagtcccgagtagaggaagaacttccgaaggtcagggaggctgcagaaaatgaattgagaaagcagcagagaaatgtagaagatatctctctgcagaagataagggctgaaagtgaagccaagcagtaccgcagggaacttgaaaccattgtgagagagaaggaagccgctgaaagagaactggagcgggtgaggcagctcaccatagaggccgaggctaaaagagctgccgtggaagagaacctcctgaattttcgcaatcagttggaggaaaacacctttaccagacgaacactggaagatcatcttaaaagaaaagatttaagtctcaatgatttggagcaacaaaaaaataaattaatggaagaattaagaagaaagagagacaatgaggaagaactcttgaagctgataaagcagatggaaaaagaccttgcatttcagaaacaggtagcagagaaacagttgaaagaaaagcagaaaattgaattggaagcaagaagaaaaataactgaaattcagtatacatgtagagaaaatgcattgccagtgtgtccgatcacacaggctacatcatgcagggcagtaacgggtctccagcaagaacatgacaagcagaaagcagaagaactcaaacagcaggtagatgaactaacagctgccaatagaaaggctgaacaagacatgagagagctgacatatgaacttaatgccctccagcttgaaaaaacgtcatctgaggaaaaggctcgtttgctaaaagataaactagatgaaacaaataatacactcagatgccttaagttggagctggaaaggaaggatcaggcggagaaagggtattctcaacaactcagagagcttggtaggcaattgaatcaaaccacaggtaaagctgaagaagccatgcaagaagctagtgatctcaagaaaataaagcgcaattatcagttagaattagaatctcttaatcatgaaaaagggaaactacaaagagaagtagacagaatcacaagggcacatgctgtagctgagaagaatattcagcatttaaattcacaaattcattcttttcgagatgagaaagaattagaaagactacaaatctgccagagaaaatcagatcatctaaaagaacaatttgagaaaagccatgagcagttgcttcaaaatatcaaagctgaaaaagaaaataatgataaaatccaaaggctcaatgaagaattggagaaaagtaatgagtgtgcagagatgctaaaacaaaaagtagaggagcttactaggcagaataatgaaaccaaattaatgatgcagagaattcaggcagaatcagagaatatagttttagagaaacaaactatccagcaaagatgtgaagcactgaaaattcaggcagatggttttaaagatcagctacgcagcacaaatgaacacttgcataaacagacaaaaacagagcaggattttcaaacaaaaattaaatgcctagaagaagacctggcgaaaagtcaaaatttggtaagtgaatttaagcaaaagtgtgaccaacagaacattatcatccagaataccaagaaagaagttagaaatctgaatgcggaactgaatgcttccaaagaagagaagcgacgcggggagcagaaagttcagctacaacaagctcaggtgcaagagttaaataacaggttgaaaaaagtacaagacgaattacacttaaagaccatagaggagcagatgacccacagaaagatggttctgtttcaggaagaatctggtaaattcaaacaatcagcagaggagtttcggaagaagatggaaaaattaatggagtccaaagtcatcactgaaaatgatatttcaggcattaggcttgactttgtgtctcttcaacaagaaaactctagagcccaagaaaatgctaagctttgtgaaacaaacattaaagaacttgaaagacagcttcaacagtatcgtgaacaaatgcagcaagggcagcacatggaagcaaatcattaccaaaaatgtcagaaacttgaggatgagctgatagcccagaagcgtgaggttgaaaacctgaagcaaaaaatggaccaacagatcaaagagcatgaacatcaattagttttgctccagtgtgaaattcaaaaaaagagcacagccaaagactgtaccttcaaaccagattttgagatgacagtgaaggagtgccagcactctggagagctgtcctctagaaacactggacaccttcacccaacacccagatcccctctgttgagatggactcaagaaccacagccattggaagagaagtggcagcatcgggttgttgaacagatacccaaagaagtccaattccagccaccaggggctccactcgagaaagagaaaagccagcagtgttactctgagtacttttctcagacaagcaccgagttacagataacttttgatgagacaaaccccattacaagactgtctgaaattgagaagataagagaccaagccctgaacaattctagaccacctgttaggtatcaagataacgcatgtgaaatggaactggtgaaggttttgacacccttagagatagctaagaacaagcagtatgatatgcatacagaagtcacaacattaaaacaagaaaagaacccagttcccagtgctgaagaatggatgcttgaagggtgcagagcatctggtggactcaagaaaggggatttccttaagaagggcttagaaccagagaccttccagaactttgatggtgatcatgcatgttcagtcagggatgatgaatttaaattccaagggcttaggcacactgtgactgccaggcagttggtggaagctaagcttctggacatgagaacaattgagcagctgcgactcggtcttaagactgttgaagaagttcagaaaactcttaacaagtttctgacgaaagccacctcaattgcagggctttacctagaatctacaaaagaaaagatttcatttgcctcagcggccgagagaatcataatagacaaaatggtggctttggcatttttagaagctcaggctgcaacaggttttataattgatcccatttcaggtcagacatattctgttgaagatgcagttcttaaaggagttgttgaccccgaattcagaattaggcttcttgaggcagagaaggcagctgtgggatattcttattcttctaagacattgtcagtgtttcaagctatggaaaatagaatgcttgacagacaaaaaggtaaacatatcttggaagcccagattgccagtgggggtgtcattgaccctgtgagaggcattcgtgttcctccagaaattgctctgcagcaggggttgttgaataatgccatcttacagtttttacatgagccatccagcaacacaagagttttccctaatcccaataacaagcaagctctgtattactcagaattactgcgaatgtgtgtatttgatgtagagtcccaatgctttctgtttccatttggggagaggaacatttccaatctcaatgtcaagaaaacacatagaatttctgtagtagatactaaaacaggatcagaattgaccgtgtatgaggctttccagagaaacctgattgagaaaactatatatcttgaactttcagggcagcaatatcagtggaaggaagctatgttttttgaatcctatgggcattcttctcatatgctgactgatactaaaacaggattacacttcaatattaatgaggctatagagcagggaacaattgacaaagccttggtcaaaaagtatcaggaaggcctcatcacacttacagaacttgctgattctttgctgagccggttagtccccaagaaagatttgcacagtcctgttgcagggtattggctgactgctagtggggaaaggatctctgtactaaaagcctcccgtagaaatttggttgatcggattactgccctccgatgccttgaagcccaagtcagtacagggggcataattgatcctcttactgtcaaaaagtaccgggtggccgaagctttgcatagaggcctggttgatgaggggtttgcccagcagctgcgacagtgtgaattagtaatcacagggattggccatcccatcactaacaaaatgatgtcagtggtggaagctgtgaaggcaaatattataaataaggaaatgggaatccgatgtttggaatttcagtacttgacaggagggttgatagagccacaggttcactctcggttatcaatagaagaggctctccaagtaggtattatagatgtcctcattgccacaaaactcaaagatcaaaagtcatatgtcagaaatataatatgccctcagacaaaaagaaagttgacatataaagaagccttagaaaaacctgattttgatttccacacaggacttaaactgttagaagtatctgagcccctgatgacaggaatttctagcctctactattcttcctaatgggacatgtttaaataactgtgcaaggggtgatgcaggctggttcatgccactttttcagagtatgatgatatcggctacatatgcagtctgtgaattatgtaacatactctatttcttgagggctgcaaattgctaagtgctcaaaatagagtaagttttaaattgaaaattacataagatttaatgcccttcaaatggtttcatttagccttgagaatggttttttgaaacttggccacactaaaatgtttttttttttacgtagaatgtgggataaacttgatgaactccaagttcacagtgtcatttcttcagaactccccttcattgaatagtgatcatttattaaatgataaattgcactcgctgaaagagcacgtcatgaagcaccatggaatcaaagagaaagatataaattcgttcccacagccttcaagctgcagtgttttagattgcttcaaaaaatgaaaaagttttgcctttttctgtatatagtgaccttctttgcatattaaaatgtttaccacaatgtcccatttctagttaagtcttcgcacttgaaagctaacattatgaatattatgtgttggaggaggggaaggattttcttcattctgtgtattttccttacatgtacagtagacgttctctattctatcagccttctatggtacctttttgtcaggacaattaggattgtaatgctaatgcaaaggcagcaattcaaagatcttctagtgcctcatgaataaagttgagatttaaaatttgtaacattgatggaacagctgggaggttagaccaatcattaaggaatgtatgccatacctttctttgctaccataaacattttggaggtgcatctgctatgtgacatggtaaatatggttaagtgaatgaataaaatgttttagtaacctgtgtcggattccgcggaat

SEQ ID NO: 32 - DNA, 5943 bp, homo sapiens
  CDS (191)..(2641)
gcagcgtgtagtgcgagtggggcggacgcgcgcagcccgcccgcccggcgaccagcaaggagttggcatcctttggaagagttcgtgaaagctttctgcccagagctcctggaccaatgcatcttcccaccaccttaaaccactgagcagttcagagccccagttgcagacgacttgtcctgccaccaccatgagttctgaatgtgatggtggttccaaagctgtgatgaatggcttggcacctggcagcaatgggcaagacaaagacatggatcctacaaaaatctgcactgggaagggagcggtgactctccgggcctcgtcttcctacagggaaaccccaagcagtagccctgcgagccctcaggaaacccggcaacacgaaagcaaaccaggtctggagccagagccttcttcagcagatgagtggaggctttcttccagtgctgatgccaatggaaatgcccagccctcttcactcgctgccaagggctacagaagtgtgcatcccaaccttccttctgacaagtcccaggatgccacttcctccagtgcagcccagccggaggtaatagttgtccctctctacctggttaatactgacagagggcaagaaggcactgccagacctccaacacctctggggcctcttggctgcgtccccacaatcccagcgactgcctctgccgcctcacctctgaccttcccgactctagatgatttcattccccctcatctgcagaggtggccccaccacagccagccagcccgcgcctctggctcctttgcccccattagccagacgccaccatccttctcaccaccacctccgctggtccctcctgccccggaggacctccgcagagtctcggagcctgacctcacgggagctgtttcgagtaccgattccagtcctctactaaatgaagtttcttcttcccttattggaactgattcccaagcctttccatcagttagcaagccttcatccgcctatccctccacaacgattgtcaatcctactattgtgctcttgcaacacaatcgagaacagcaaaaacgactcagtagcctttcagatcctgtctcagaaagaagagtgggagagcaggactcagcaccaacccaggaaaaacccacctcacctggcaaggctattgaaaaaagagcaaaggatgacagtaggcgggtggtgaagagcactcaggacttaagcgatgtttccatggatgaagtgggcatcccactccggaacactgagagatcaaaagactggtacaagactatgtttaaacagatccacaaactgaacagagatgatgattcagatctgtactctcccagatactcattttctgaagacacaaaatctcccctttctgtgcctcgctcaaaaagtgagatgagctacattgatggtgagaaggtagtcaagaggtcggccacactacccctcccagcccgctcttcctcactgaagtcaagctcagaaagaaatgactgggaacccccagataagaaagtagatacaagaaaatatcgtgcagagcccaagagcatttacgaatatcagcctggcaagtcttccgttctgaccaacgaaaagatgagctcagccatcagccctactccggaaatttcttcagagactcctggatatatatattcttccaacttccatgcagtgaagagggaatcagacggggctcctggggatctcactagcttggagaatgagagacaaatttataaaagtgtcttggaaggtggtgacatccctcttcagggcctgagtgggctcaagcgaccatccagctctgcttccactaaagattcagaatcgccaagacattttataccagctgattacttggaatccacggaagaatttattcgaagacgtcatgatgataaagagatgagacctgccagagccaaatttgactttaaagctcagacactaaaggagcttcctctgcagaagggagatattgtttacatttataagcaaattgatcagaactggtatgaaggagaacaccacggccgggtgggaatcttcccacgcacctacatcgagcttcttcctcctgctgagaaggcacagcccaaaaagttgacaccagtgcaggttttggaatatggagaagctattgctaagtttaactttaatggtgatacacaagtagaaatgtccttcagaaagggtgagaggatcacactgctccggcaggtagatgagaactggtacgaagggaggatcccggggacatcccgacaaggcatcttccccatcacctacgtggatgtgatcaagcgaccactggtgaaaaaccctgtggattacatggacctgcctttctcctcctccccaagtcgcagtgccactgcaagcccacagcaacctcaagcccagcagcgaagagtcacccccgacaggagtcaaacctcacaagatttatttagctatcaagcattatatagctatataccacagaatgatgatgagttggaactccgcgatggagatatcgttgatgtcatggaaaaatgtgacgatggatggtttgttggtacttcaagaaggacaaagcagtttggtacttttccaggcaactatgtaaaacctttgtatctataagaagactgaaaaccatggagattatttttattggaggaggaagcatcattcatgaaccgatctttttagttgagtcagtaggaaaattaatacagtggataaagtaagaagcaaaagacagggacagagaagtgttgtgtttaaaacccaagcctgtctaaggttactgtgtattagacagggccgaactagtgtgctgagcaaaaagaattgaagcaaattgtatttacttagccgcttctgggagccacttcagcctttcccctcccctccacttcttgggtaatctgacctgaagcatagtccaggagcagagttagccagaaatgcctcctgctgccccagccttagagagctcccatctcaatcattgagcctgaaggcttcaagcccaagaatgcaacaagacccccagcctacatttctcagctcccctggagccagctgatcctgtaacgctgctggaggtcagtctgagctaccaagactgtccctagacaaaggtggagtcccccacactgcccaagaccaaatccctcactcaacctgctgaggtgtggatggggaaacagaggcaaaactgaggcacctgatgcattcagcctgctgtgcagcagtgccattgactgccctgatgttcagagagaaacgcacacaaggtttgcccatgagaattggggagcagatggccaagcagataggttatgtctgttttctgagtgatgaagtcaggaagccctgtggctctggaggccacttgtggttcattcttttcccatatccttggcttttagaaatggttaccttcaggacagtgcagctgcatttatcagagcactattgctaagttttcttttctggcttgtgtttttctgggacagtttagaattgggaggcctattctcatagaacaccaaaaatgatgttcagtgattcatttaacatacaccaatgtactctggctgctggggggacaaccataagcaagacatgcccagggtttgccgtggctccagatctactccctgtaggagttcaaggatcacacaaacggtagtaaccagggttgtgaatctgagtacaccctggcaaggcttctcttcagactgaagcagcaattctgccactaccagcagcaaccaggacgtctgttctttgtgggggccagatcagaagagagaggcccctgtgacgcccgggctgcttggtcacaactctgtccaattcaaggatgtttatcggcctctcttagatcctgagtgagacaaatacagaaatgacccattccctgcccaccagaaactcagaggtgattggggagactgacacaggaaaatgaacttaatcaagagagactgtgatatgtgctaagaagggtgtgagggagggagagatgaattttccctggagggatcctagaaagcattgtcatattgccatctccattagctcacttttaaacaactagggtgctggaagaacctttgtctgagggtagttcatagctggaaatacttggaatattttccagagtctctaaactctcatcttcccccacagatacacatccaagctcacaaataggagtagcaattctaggtggtagggttgtgtacggaacccctggctgtctgcatatatctcagaattaccccaggaccattgtcccaaagtctagagtctttacaggtaggcaaaatttgttttcaatgcctgtgcctcagctgctgtcacaaatacccatcttaggatcccatcagcttcccatcccccaccagacagccacagtaccctcactttctccctattgttctttcaaatcctgttctcaggaaagaaactgccactaattcattcacactaaggtgtaaatgattgataataggaatgagttacctcttcccacagacatttgtttttaagtatgacagagcagggccttaatcccaagggaaaaggttatggaactggagggggtgagctttctgggtagaaggagacttcctgaatttccttaaaacccagtaagagtaagacctgttgttttggaaggtctgctccaccatctaagagcactgtttttttttttttgttgttgttgttgttttacggtctctgagggaatatagtaaaaatgcatatgcacgtgcaatttgcacggcagcatttcaccgattgtggactgtattggctaatgtgtttcctggtctttagatgcaaaccattaataacactatcttatctcatagttttttcaggggtgcttcttgattagtagggaattttgaacacctctttaaatacagctagaaaataaaaccaatttgtaaagccacatttgcatatgatgccagcctcacgcatttgtatatctccagaaattcaggtatgcctcaccaatttgcccgtctttaataaaatcttgtgttaaaatttgcatcacgtcgccttcctatgtatgacgaaacaagaaacagagatttccaattgctcttttgtcttcagacatttagtaatataaagtacctatttttatgctgaaatgtttatacaggtttattaatagcaagtgcaactaactggcggcatgccttgcaacacattttgatatattagccatgcttccgggtaaaggcaagccccaaactccttatcttttgcagtctctctgggatcagtaaaagaaaaaaaaaataatgtgcttaagaagtgggactgtaaatatgtatatttaactttgtatagcccatgtacctaccttgtatagaaaaataattttaaaaatttgaatggaagggggtaaaggaagtcatgaagtttttttgcatttttatttaaatgaaggaattccaaataactcacctacagatttttagcacaaaaatagccattgtaaagtgttaaaatttacgataagtattctattggggaggaaaggtaactctgatctcagttacagtttttttttcctttttaatttcattattttgggtttttggtttttgcagtcctatttatctgcagtcgtattaagtcctattgctagaataggttactacaaaaaaggttatattctgaaagaaaaataactgacattatatataaccaattaatttaaagtattgccatttaaattacacactgagagcatgtcctatgcagacatagatttttctgttcatttatttttcttcattgcagtggattgatttgataaatagatgtgttgaattactacatttgctgtacatattatttaataaactttattcagaattgcgtggcaaaaaaaaaaaaaaaaa

SEQ ID NO: 33 - protein, 816 aa, homo sapiens
MSSECDGGSKAVMNGLAPGSNGQDKDMDPTKICTGKGAVTLRASSSYRETPSSSPASPQETRQHESKPGLEPEPSSADEWRLSSSADANGNAQPSSLAAKGYRSVHPNLPSDKSQDATSSSAAQPEVIVVPLYLVNTDRGQEGTARPPTPLGPLGCVPTIPATASAASPLTFPTLDDFIPPHLQRWPHHSQPARASGSFAPISQTPPSFSPPPPLVPPAPEDLRRVSEPDLTGAVSSTDSSPLLNEVSSSLIGTDSQAFPSVSKPSSAYPSTTIVNPTIVLLQHNREQQKRLSSLSDPVSERRVGEQDSAPTQEKPTSPGKAIEKRAKDDSRRVVKSTQDLSDVSMDEVGIPLRNTERSKDWYKTMFKQIHKLNRDDDSDLYSPRYSFSEDTKSPLSVPRSKSEMSYIDGEKVVKRSATLPLPARSSSLKSSSERNDWEPPDKKVDTRKYRAEPKSIYEYQPGKSSVLTNEKMSSAISPTPEISSETPGYIYSSNFHAVKRESDGAPGDLTSLENERQIYKSVLEGGDIPLQGLSGLKRPSSSASTKDSESPRHFIPADYLESTEEFIRRRHDDKEMRPARAKFDFKAQTLKELPLQKGDIVYIYKQIDQNWYEGEHHGRVGIFPRTYIELLPPAEKAQPKKLTPVQVLEYGEAIAKFNFNGDTQVEMSFRKGERITLLRQVDENWYEGRIPGTSRQGIFPITYVDVIKRPLVKNPVDYMDLPFSSSPSRSATASPQQPQAQQRRVTPDRSQTSQDLFSYQALYSYIPQNDDELELRDGDIVDVMEKCDDGWFVGTSRRTKQFGTFPGNYVKPLYL

SEQ ID NO: 34 - DNA, 572 bp, homo sapiens
tttttttttttgactcttccctcctcatttattttggaatgtgctagaaacagcttgaaacatccctttaatagcttcccggcctcacgagtgttgaatgacatgacgaattctccttcatagaaggtacaggtgaaccagaactggaggggcatttgggatccttccttcttcagaaagtgcgatcgcatcaagatgcatgtggttttcagtagaactggcccatgtttcttgggagcgaggtgtccaaaccactgttcatccatatttcctgaatgatttgctccctgcgctcaatcctttctgcgagctctgcagcttctatggaactgtatataggatatgaagtcctacagaaccccgacagttcccctgggaaatcagactcagaggggtcctcctcctcttcttcatcctcgctgtcctcctcgaccttgtcacttcctggcaccagctgctccctccccagctgcaagtcgcggaagtccttggcacaggactctctgatgaccagggcgcacagtgtgagggccaggccgatgcagacactggacacgaacagcagggcagct

SEQ ID NO: 35 - DNA, 5678 bp, homo sapiens
  CDS (101)..(5467)
taaccacgaaagaggaatcgatgctcagcttttagttgcacttcctaaagttgcagaattaagacaaatctttgaaccaaagaagaaagaattcttagaaatgaaaagaaaagaaagaattgccaggcgcctggaagggattgaaaatgacactcagcccatcctcttgcagagctgcacaggattggtgactcaccgcctgctggaggaagacacccctcgatacatgagagccagcgaccctgccagcccccacatcggccgatcaaatgaagaggaggaaacttctgattcttctctagaaaagcaaactcgatccaaatactgcacagaaacctccggtgtccacggtgactcaccctatggttcgggtaccatggacacccacagtctggagtccaaagccgaaagaattgcaaggtacaaagcagaaagaaggcgacagctggcagagaagtatgggctgactctggatcccgaggccgactccgagtatttatcccgctataccaagtccaggaaggagcctgatgctgtcgagaagcggggaggaaaaagtgacaaacaggaagagtcaagcagagatgckagttctctgtaccccgggaccgagacgatggggctcaggacctgtgccggtgaatccaaggactatgccctccatgtgggtgacggctcttccgacccggaggtgctgctgaacatagaaaaccaaagacgaggtcaagagctgagtgccacccggcaggcccatgacctgtccccagcagccgagagttcctcgaccttctctttctctgggcgagactcctccttcactgaagtgccacggtcccccaagcacgcccacagctcctccctgcagcaggcagcctcccggagcccctcctttggtgacccacagctatcccctgaggcccgacccaggtgcacttcacattcagaaacgccaactgtcgatgatgaagaaaaggtggatgaacgagccaagctgagcgtcgccgccaagaggttgcttttcagggagatggaaaaatcttttgatgaacaaaatgttccaaagcgacgctcaagaaacacagctgtggagcagaggctacgccgtctgcaggacaggtccctcacccagcccatcaccactgaagaggtggtcatcgcagccacattgcaggcctctgctcaccaaaaggccttagccaaggaccagacaaatgagggcaaagagcttgctgagcaaggagaacctgattcctccactctaagcttggccgaaaagttggccttgtttaacaaattgtcccagccagtctcaaaagcgatttctacccggaacagaatagacacgagacagaggagaatgaacgctcgctatcaaactcagccagtcacactgggagaggtggagcaggtgcagagtggaaagctcattcctttctcacctgccgtgaacacatcagtgtctaccgtagcatccacggttgctccaatgtatgccggagatcttcgcacaaagccacctcttgaccacaatgcaagtgccactgactataagttttcttcttcaatagaaaattcggactctccagttagaagcattctgaaatcgcaagcttggcagcctttggtagagggtagcgagaacaagggaatgttgagagaatatggagagacagaaagcaagagagctttgacaggtcgagacagtgggatggagaagtatgggtcctttgaggaagcagaagcatcctaccccatcctgaaccgagccagggaaggagacagccataaggaatctaaatatgctgttcccagaagaggaagcctggaacgggcgaaccctcccatcacccacctcggggatgaaccgaaggaattttccatggctaaaatgaatgcacaaggaaacttggacttgagggacaggctgccctttgaagagaaggtggaggtggagaatgttatgaaaaggaagttttcactaagagcggcagagttcggggagcccacttccgagcagacggggacagctgctgggaaaactattgctcaaaccacagcccccgtgtcctggaagccccaggattcttcggaacagccacaggagaagctctgcaagaatccatgtgcgatgtttgctgctggagagatcaaaacgccgacaggggagggccttcttgactcacccagcaaaaccatgtctattaaagaaagattggcactgttgaagaaaagcggggaggaagattggagaaacagactcagcaggaggcaggagggcggcaaggcgccggccagcagcctgcacacccaggaagcagggcggtccctcatcaagaagcgggtcacagaaagtcgagagagccaaatgacgattgaggagaggaagcagctcatcactgtgagagaggaggcctggaagacgagaggcagaggagcggccaacgactcgacccagttcactgtggctggcaggatggtgaagaaaggtttggcgtcacctactgccataaccccagtagcctcagccatttgcggtaaaacaagaggcaccacacccgtttccaaacccctggaagatatcgaagccagaccagatatgcagttagaatcggacctgaagttggacaggctggaaacctttctaagaaggctgaataacaaagttggcgggatgcacgaaacggtgctcactgtcaccggcaaatctgtgaaggaggtgatgaagccagatgatgatgaaacctttgccaaattttaccgcagcgtggattataatatgccaagaagtcctgtggagatggatgaggacttcgatgtcattttcgatccttatgcacccaaattgacgtcttccgtggccgagcacaagcgggcagttaggcccaagcgccgggttcaggcctccaaaaaccccctgaaaatgctggcggcaagagaagatctccttcaggaatacactgagcagagattaaacgttgccttcatggagtcaaagcggatgaaagtagaaaagatgtcttccaactccaacttctcagaagtcaccctggcgggtttagccagtaaagaaaacttcagcaacgtcagcctgcggagcgtcaacctgacggaacagaactctaacaacagcgccgtgccctacaagaggctgatgctgttgcagattaaaggaagaagacatgtgcagaccaggctggtggaacctcgagcttcggcgctcaacagtggggactgcttcctcctgctctctccccactgctgcttcctgtgggtaggagagtttgcaaacgtcatagaaaaggcgaaggcctcagaacttgcaactttaattcagacaaagagggaacttggttgtagagctacttatatccaaaccattgaagaaggaattaatacacacactcatgcagccaaagacttctggaagcttctgggtggccaaaccagttaccaatctgctggagacccaaaagaagatgaactctatgaagcagccataatagaaactaactgcatttaccgtctcatggatgacaaacttgttcctgatgacgactactgggggaaaattccgaagtgctcccttctgcaacccaaagaggtactggtgtttgattttggtagtgaagtttacgtatggcatgggaaagaagtcacattagcacaacgaaaaatagcatttcagctggcaaagcacttatggaatggaacctttgactatgagaactgtgacatcaatcccctggatcctggagaatgcaatccgcttatccccagaaaaggacaggggcggcccgactgggcgatatttgggagacttactgaacacaatgagacgattttgttcaaagagaagtttctggattggacggaactgaagagatcgaatgagaagaaccccggggaacttgcccagcacaaggaagaccccaggactgatgtcaaggcatacgatgtgacacggatggtgtccatgccccagacgacagcaggcaccatcctggacggagtgaacgtcggccgtggctatggcctggtggaaggacacgacaggaggcagtttgagatcaccagcgtttccgtggatgtctggcacatcctggaattcgactatagcaggctccccaaacaaagcatcgggcagttccatgagggggatgcctatgtggtcaagtggaagttcatggtgagcacggcagtgggaagtcgccagaagggagagcactcggtgagggcagccggcaaagagaagtgcgtctacttcttctggcaaggccggcactccaccgtgagtgagaagggcacgtcggcgctgatgacggtggagctggacgaggaaaggggggcccaggtccaggttctccagggaaaggagcccccctgtttcctgcagtgtttccagggggggatggtggtgcactcggggaggcgggaagaggaagaagaaaatgtgcaaagtgagtggcggctgtactgcgtgcgtggagaggtgcccgtggaagggaatttgctggaagtggcctgtcactgtagcagcctgaggtccagaacttccatggtggtgcttaacgtcaacaaggccctcatctacctgtggcacggatgcaaagcccaggcccacacgaaggaggtcggaaggaccgctgcgaacaagatcaaggaacaatgtcccctggaagcaggactgcatagtagcagcaaagtcacaatacacgagtgtgatgaaggctccgagccactcggattctgggatgccttaggaaggagagacaggaaagcctacgattgcatgcttcaagatcctggaagttttaacttcgcgccccgcctgttcatcctcagcagctcctctggggattttgcagccacagagtttgtgtaccctgcccgagccccctctgtggtcagttccatgcccttcctgcaggaagatctgtacagcgcgccccagccagcacttttccttgttgacaatcaccacgaggtgtacctctggcaaggctggtggcccatcgagaacaagatcactggttccgcccgcatccgctgggcctccgaccggaagagtgcgatggagactgtgctccagtactgcaaaggaaaaaatctcaagaaaccagcccccaagtcttaccttatccacgctggtctggagcccctgacattcaccaatatgtttcccagctgggagcacagagaggacatcgctgagatcacagagatggacacggaagtttccaatcagatcaccctcgtggaagacgtcttagccaagctctgtaaaaccatttacccgctggccgacctcctggccaggccactcccggagggggtcgatcctctgaagcttgagatctatctcaccgacgaagacttcgagtttgcactagacatgacgagggatgaatacaacgccctgcccgcctggaagcaggtgaacctgaagaaagcaaaaggcctgttctgagtggggagacgccagaggagcctcacggtcacgtccaacaacaccactgcaccagggaaatggatatatatttttggactggtgtttttcacaaagtatttttcaatcagagttttcagaacctgacattgttaaagatactgcttgtcccggagttgtgtattttgtaaatgttcaagggaactgtttggaaacttctttccaccattca

SEQ ID NO: 36 - protein, 1788 aa, homo sapiens
MKRKERIARRLEGIENDTQPILLQSCTGLVTHRLLEEDTPRYMRASDPASPHIGRSNEEEETSDSSLEKQTRSKYCTETSGVHGDSPYGSGTMDTHSLESKAERIARYKAERRRQLAEKYGLTLDPEADSEYLSRYTKSRKEPDAVEKRGGKSDKQEESSRDASSLYPGTETMGLRTCAGESKDYALHVGDGSSDPEVLLNIENQRRGQELSATRQAHDLSPAAESSSTFSFSGRDSSFTEVPRSPKHAHSSSLQQAASRSPSFGDPQLSPEARPRCTSHSETPTVDDEEKVDERAKLSVAAKRLLFREMEKSFDEQNVPKRRSRNTAVEQRLRRLQDRSLTQPITTEEVVIAATLQASAHQKALAKDQTNEGKELAEQGEPDSSTLSLAEKLALFNKLSQPVSKAISTRNRIDTRQRRMNARYQTQPVTLGEVEQVQSGKLIPFSPAVNTSVSTVASTVAPMYAGDLRTKPPLDHNASATDYKFSSSIENSDSPVRSILKSQAWQPLVEGSENKGMLREYGETESKRALTGRDSGMEKYGSFEEAEASYPILNRAREGDSHKESKYAVPRRGSLERANPPITHLGDEPKEFSMAKMNAQGNLDLRDRLPFEEKVEVENVMKRKFSLRAAEFGEPTSEQTGTAAGKTIAQTTAPVSWKPQDSSEQPQEKLCKNPCAMFAAGEIKTPTGEGLLDSPSKTMSIKERLALLKKSGEEDWRNRLSRRQEGGKAPASSLHTQEAGRSLIKKRVTESRESQMTIEERKQLITVREEAWKTRGRGAANDSTQFTVAGRMVKKGLASPTAITPVASAICGKTRGTTPVSKPLEDIEARPDMQLESDLKLDRLETFLRRLNNKVGGMHETVLTVTGKSVKEVMKPDDDETFAKFYRSVDYNMPRSPVEMDEDFDVIFDPYAPKLTSSVAEHKRAVRPKRRVQASKNPLKMLAAREDLLQEYTEQRLNVAFMESKRMKVEKMSSNSNFSEVTLAGLASKENFSNVSLRSVNLTEQNSNNSAVPYKRLMLLQIKGRRHVQTRLVEPRASALNSGDCFLLLSPHCCFLWVGEFANVIEKAKASELATLIQTKRELGCRATYIQTIEEGINTHTHAAKDFWKLLGGQTSYQSAGDPKEDELYEAAIIETNCIYRLMDDKLVPDDDYWGKIPKCSLLQPKEVLVFDFGSEVYVWHGKEVTLAQRKIAFQLAKHLWNGTFDYENCDINPLDPGECNPLIPRKGQGRPDWAIFGRLTEHNETILFKEKFLDWTELKRSNEKNPGELAQHKEDPRTDVKAYDVTRMVSMPQTTAGTILDGVNVGRGYGLVEGHDRRQFEITSVSVDVWHILEFDYSRLPKQSIGQFHEGDAYVVKWKFMVSTAVGSRQKGEHSVRAAGKEKCVYFFWQGRHSTVSEKGTSALMTVELDEERGAQVQVLQGKEPPCFLQCFQGGMVVHSGRREEEEENVQSEWRLYCVRGEVPVEGNLLEVACHCSSLRSRTSMVVLNVNKALIYLWHGCKAQAHTKEVGRTAANKIKEQCPLEAGLHSSSKVTIHECDEGSEPLGFWDALGRRDRKAYDCMLQDPGSFNFAPRLFILSSSSGDFAATEFVYPARAPSVVSSMPFLQEDLYSAPQPALFLVDNHHEVYLWQGWWPIENKITGSARIRWASDRKSAMETVLQYCKGKNLKKPAPKSYLIHAGLEPLTFTNMFPSWEHREDIAEITEMDTEVSNQITLVEDVLAKLCKTIYPLADLLARPLPEGVDPLKLEIYLTDEDFEFALDMTRDEYNALPAWKQVNLKKAKGLF

SEQ ID NO: 37 - DNA, 604 bp, homo sapiens
  misc_feature (505)..(505): n is a, c, g, or t
ttttgacatttttttatttttattgaaaataaaaaagtcatggtgctttcttcctcataagtggcatatggacacccccagtgctgccggtctcgcttccggtacaaaagtctcagcaggaaaccaaccttaatggcttgttgatggataagacggcatcaacttgtacatttgcccattgagagaactcccaggagcaactgtcctctctccagtacacgcggcacgttgctaagaaggcagatttcaggatattcacattcatgcattacgtatctcacactacctgggcagtttaactcatactttgtacagatgcagagagtacagtagttgtatttatatatatatatatgtagagatctctttaaatatatatagctatatataatatatatgtttatatgtttacacctattagtctttcttccaaaacttcctttagaagcttcttaggaacacctgaaacctctgggagatcctatgatctgagttccaaagttgnggtcaactgtgagacggctggaaaagatctatttgagaaaatgagaagggaagtgcatttcattcttgacctcagggctcggactagcctgaggatcgg

SEQ ID NO: 38 - DNA, 2039 bp, homo sapiens
  CDS (130)..(633)
cctttgtcattctagctgcctgctgcctccgcagcgtccccccagctctccctgtgctaactgcctgcaccttggacagagcgggtgcgcaaatcagaaggattagttgggacctgccttggcgaccccatggcatcccccagaaccgtaactattgtggccctctcagtggccctgggactcttctttgttttcatggggactatcaagctgacccccaggctcagcaaggatgcctacagtgagatgaaacgtgcttacaagagctatgttcgagccctccctctgctgaagaaaatggggatcaattccattctcctccgaaaaagcattggtgcccttgaagtggcctgtggcatcgtcatgacccttgtgcctgggcgtcccaaagatgtggccaacttcttcctactgttgctggtgttggctgtgctcttcttccaccagctggtcggtgatcctctcaaacgctacgcccatgctctggtgtttggaatcctgctcacttgccgcctgctgattgctcgcaagcccgaagaccggtcttctgagaagaagcctttgccagggaatgctgaggagcaaccctccttatatgagaaggcccctcagggcaaagtgaaggtgtcatagaaaagtggaagtgcaaagagtggaccttccaggcagttgcgtccatgacaccaggaagatgtcagtgtgtgtttttcatttgatttatttatcttggggaaagtgaaaaatgtaatctgcaagttaatgaccctattggcttgtgtacatctatatgctaaaatgacttccccacattgacatttgtgcgccacctttaatcactctggggcaactctcacatcttgctgcatgtacatgtatacggctactattgaagtgtaattgtgagatggactccaacaagcatgtgactgtgagattgtgtgtgggaaaatgtatttaactactctgtgtgtgtgtgtgtgtgtgtgtgcgcacgcgcgcacgcgcacacactcacgcacacacaagcagagaaggcgctgatcttgaactaatcctgcacaggcatccttccctttatagattgattccagcaaaggcggaataaaacaaatttcctatgaagagaatcctgatatgaaacaagtcatgtagtctcatggccgggaatctctccacagatactaacaacttaaacttactactttaggagaaaaaaaaaaacattcaatttcggacactgagttatatatgaaattaattaggctctagtccaacagttgtttacattttaaatagtccatattgaatttaattaaaacaagggatgcatgcagtcaaattgatagtttaattcttcaagtgataatataggaagtttcaccttgcctttgtccaagccccacctattaaaaccctttactcacagtttgaaactgaagcagtaaacttgtttccagacatctttttcagattgtcttaagcccaaagttgcctcacttccactattctcagcagccaaccaggatttggcagctgctccactgttacggttgagggaacagggatcagccctgttagaagtctgtgagcctcaaactctacctgttctctgcaatcatccaaaatttgaaaaagaagctatatccagtgtttcactgccaaacagattcactactcttactgattcttcactgagctttgctagtataagcagagttccaagtctcccctagggttgtctctacatttctttatcattccagtgggtagggtttagctgggggaaggacatttcataagggttagttggactgagcagtatggacatttgcttttttcattacgtactgttgtttttccttgttaggtgtgctttggtggttttaatattattgtgccagggatggggaaatggggggggttgtgtgggaagagtacttattattgtgttttcttcagtgtaattgttcttggtaattgatacctctctgttttatttctctcattctttcaaaataaaactttttgaaatttggagg

SEQ ID NO: 39 - protein, 167 aa, homo sapiens
MASPRTVTIVALSVALGLFFVFMGTIKLTPRLSKDAYSEMKRAYKSYVRALPLLKKMGINSILLRKSIGALEVACGIVMTLVPGRPKDVANFFLLLLVLAVLFFHQLVGDPLKRYAHALVFGILLTCRLLIARKPEDRSSEKKPLPGNAEEQPSLYEKAPQGKVKVS

SEQ ID NO: 40 - DNA, 1646 bp, homo sapiens
  CDS (75)..(509)
acttcccggtggagtgaggaacccagcaacacgctcctgacttcccttcccaaggactcgacctgagaaccgccatgtactcggagatccagagggagcgggcagacattgggggcctgatggcccggccagaatacagagagtggaatccggagctcatcaagcccaagaagctgctgaaccccgtgaaggcctctcggagtcaccaggagctccaccgggagctgctcatgaaccacagaaggggccttggtgtggacagcaagccagagctgcagcgtgtcctagagcaccgccggcggaaccagctcatcaagaagaagaaggaggagctggaagccaagcggctgcagtgcccctttgagcaggagctgctgagacggcagcagaggctgaaccagctggaaaaaccaccagagaaggaagaggatcacgcccccgagtttattaaagtcagggaaaacctgcggagaattgccacactgaccagcgaagagagagagctgtagggccagctgccgggctcaggccactgcccaccctggcctggacagcctccttcagcccttctgtacctggcagccctgggccccaggccctgggacgtctgtgatgttcccacctgcttctgtagaaatgtgtcaccccagagggcctggctctccctgggaggctggggcccctaagctcctaggttttccttccaagcacccagccctcctgctccaagagggataacctgcacccctccctgcaaggggttcagagcccagcacaggagctttctctggcagaattgaggaggaagaggtggccctctgacttgacaagccttctgttctgcccaggccttcccaccaggaatctccgaggctccccagggccccgcttctccgtacaccccagctcctaggtctcagagaactcccccacctgtggttttacctgcagccagcagagcttagcttcaaggacacctgccttcaaagccactgaggggaggaagggcagggcagactgcaggtggccttgttgctggcatcccggccaggtgggcggggactaacaaagacagctgtttagggtcttctcccctcacccatgctttcatcatcccctccgcacagcctccccgtccaggccttctaaccacacctacccagggctgccgcattcctgcactcagaagtctgcagcggtgcctcacaaacttgattgtgcataaaaatcactggggatcttgttaatacagattctaactcaatagatctgggagatcctgcatttctaacaagctcccaggtaaggcggaggctgctggtgtgaggaccatgctgtgagcagcagggcgagagtgcccagggctgatatatattggaaatatcacccctgaagccatcgctggcccccacctcctgtggactgatgccccagggattcccaccccacttctgcaaccccaggtatccttcattatccaccccatcccagactcccaccccagggattgcccgtgaagactttggcctagcaaattgtgttggttatgtgagtgttgttttaatcagagatgtacatgattgccaatctgcatttcttaccagtgtgaccacactgttacgatgcaattctagccaaaaaaaaaa

SEQ ID NO: 41 - protein, 144 aa, homo sapiens
MYSEIQRERADIGGLMARPEYREWNPELIKPKKLLNPVKASRSHQELHRELLMNHRRGLGVDSKPELQRVLEHRRRNQLIKKKKEELEAKRLQCPFEQELLRRQQRLNQLEKPPEKEEDHAPEFIKVRENLRRIATLTSEEREL

SEQ ID NO: 42 - DNA, 228 bp, homo sapiens
tttttttttttttttttaggctttattgaatttatttgctttgcatatatcttaaatcaaaaaagttaaagaagtagaaaagttgtcagaatatgcctttaggcatttaaaaagaacttaatctcttcatttaaaaacagaactttgttgttaactgtggaaaagaaattgttattggagagttctcagagtgagaatagctgaatacaggttcactgtgaaaaaaag

SEQ ID NO: 43 - DNA, 2797 bp, homo sapiens
  CDS (351)..(1775)
ttccccagcattcgagaaactcctctctactttagcacggtctccagactcagccgagagacagcaaactgcagcgcggtgagagagcgagagagagggagagagagactctccagcctgggaactataactcctctgcgagaggcggagaactccttccccaaatcttttggggacttttctctctttacccacctccgcccctgcgaggagttgaggggccagttcggccgccgcgcgcgtcttcccgttcggcgtgtgcttggcccggggaaccgggagggcccggcgatcgcgcggcggccgccgcgagggtgtgagcgcgcgtgggcgcccgccgagccgaggccatggtgcagcaaaccaacaatgccgagaacacggaagcgctgctggccggcgagagctcggactcgggcgccggcctcgagctgggaatcgcctcctcccccacgcccggctccaccgcctccacgggcggcaaggccgacgacccgagctggtgcaagaccccgagtgggcacatcaagcgacccatgaacgccttcatggtgtggtcgcagatcgagcggcgcaagatcatggagcagtcgcccgacatgcacaacgccgagatctccaagcggctgggcaaacgctggaagctgctcaaagacagcgacaagatccctttcattcgagaggcggagcggctgcgcctcaagcacatggctgactaccccgactacaagtaccggcccaggaagaaggtgaagtccggcaacgccaactccagctcctcggccgccgcctcctccaagccgggggagaagggagacaaggtcggtggcagtggcgggggcggccatgggggcggcggcggcggcgggagcagcaacgcggggggaggaggcggcggtgcgagtggcggcggcgccaactccaaaccggcgcagaaaaagagctgcggctccaaagtggcgggcggcgcgggcggtggggttagcaaaccgcacgccaagctcatcctggcaggcggcggcggcggcgggaaagcagcggctgccgccgccgcctccttcgccgccgaacaggcgggggccgccgccctgctgcccctgggcgccgccgccgaccaccactcgctgtacaaggcgcggactcccagcgcctcggcctccgcctcctcggcagcctcggcctccgcagcgctcgcggccccgggcaagcacctggcggagaagaaggtgaagcgcgtctacctgttcggcggcctgggcacgtcgtcgtcgcccgtgggcggcgtgggcgcgggagccgaccccagcgaccccctgggcctgtacgaggaggagggcgcgggctgctcgcccgacgcgcccagcctgagcggccgcagcagcgccgcctcgtcccccgccgccggccgctcgcccgccgaccaccgcggctacgccagcctgcgcgccgcctcgcccgccccgtccagcgcgccctcgcacgcgtcctcctcggcctcgtcccactcctcctcttcctcctcctcgggctcctcgtcctccgacgacgagttcgaagacgacctgctcgacctgaaccccagctcaaactttgagagcatgtccctgggcagcttcagttcgtcgtcggcgctcgaccgggacctggattttaacttcgagcccggctccggctcgcacttcgagttcccggactactgcacgcccgaggtgagcgagatgatctcgggagactggctcgagtccagcatctccaacctggttttcacctactgaagggcgcgcaggcagggagaagggccggggggggtaggagaggagaaaaaaaaagtgaaaaaaagaaacgaaaaggacagacgaagagtttaaagagaaaagggaaaaaagaaagaaaaagtaagcagggctcgttcgcccgcgttctcgtcgtcggatcaaggagcgcggcggcgttttggacccgcgctcccatcccccaccttcccgggccggggacccactctgcccagccggagggacgcggaggaggaagagggtagacaggggcgacctgtgattgttgttattgatgttgttgttgatggcaaaaaaaaaaagcgacttcgagtttgctcccctttgcttgaagagaccccctcccccttccaacgagcttccggacttgtctgcacccccagcaagaaggcgagttagttttctagagacttgaaggagtctcccccttcctgcatcaccaccttggttttgttttattttgcttcttggtcaagaaaggaggggagaacccagcgcacccctccccccctttttttaaacgcgtgatgaagacagaaggctccggggtgacgaatttggccgatggcagatgttttgggggaacgccgggactgagagactccacgcaggcgaattcccgtttggggcctttttttcctccctcttttccccttgccccctctgcagccggaggaggagatgttgaggggaggaggccagccagtgtgaccggcgctaggaaatgacccgagaaccccgttggaagcgcagcagcgggagctaggggcgggggcggaggaggacacgaactggaagggggttcacggtcaaactgaaatggatttgcacgttggggagctggcggcggcggctgctgggcctccgccttcttttctacgtgaaatcagtgaggtgagacttcccagaccccggaggcgtggaggagaggagactgtttgatgtggtacaggggcagtcagtggagggcgagtggtttcggaaaaaaaaaaagaaaaaaaggg

SEQ ID NO: 44 - protein, 474 aa, homo sapiens
MVQQTNNAENTEALLAGESSDSGAGLELGIASSPTPGSTASTGGKADDPSWCKTPSGHIKRPMNAFMVWSQIERRKIMEQSPDMHNAEISKRLGKRWKLLKDSDKIPFIREAERLRLKHMADYPDYKYRPRKKVKSGNANSSSSAAASSKPGEKGDKVGGSGGGGHGGGGGGGSSNAGGGGGGASGGGANSKPAQKKSCGSKVAGGAGGGVSKPHAKLILAGGGGGGKAAAAAAASFAAEQAGAAALLPLGAAADHHSLYKARTPSASASASSAASASAALAAPGKHLAEKKVKRVYLFGGLGTSSSPVGGVGAGADPSDPLGLYEEEGAGCSPDAPSLSGRSSAASSPAAGRSPADHRGYASLRAASPAPSSAPSHASSSASSHSSSSSSSGSSSSDDEFEDDLLDLNPSSNFESMSLGSFSSSSALDRDLDFNFEPGSGSHFEFPDYCTPEVSEMISGDWLESSISNLVFTY

SEQ ID NO: 45 - DNA, 1334 bp, homo sapiens
  CDS (14)..(601)
gcagacccccatcatgggcagccagagctccaaggctccccggggcgacgtgaccgccgaggaggcagcaggcgcttcccccgcgaaggccaacggccaggagaatggccacgtgaaaagcaatggagacttatcccccaagggtgaaggggagtcgccccctgtgaacggaacagatgaggcagccggggccactggcgatgccatcgagccagcaccccctagccagggtgctgaggccaagggggaggtcccccccaaggagacccccaagaagaagaagaaattctctttcaagaagcctttcaaattgagcggcctgtccttcaagagaaatcggaaggagggtgggggtgattcttctgcctcctcacccacagaggaagagcaggagcagggggagatcggtgcctgcagcgacgagggcactgctcaggaagggaaggccgcagccacccctgagagccaggaaccccaggccaagggggcagaggctagtgcagcctcagaagaagaggcagggccccaggctacagagccatccactccctcggggccggagagtggccctacaccagccagcgctgagcagaatgagtagctaggtaggggcaggtgggtgatctctaagctgcaaaaactgtgctgtccttgtgaggtcactgcctggacctggtgccctggctgccttcctgtgcccagaaaggaaggggctattgcctcctcccagccacgttccctttcctcctctccctcctgtggattctcccatcagccatctggttctcctcttaaggccagttgaagatggtcccttacagcttcccaagttaggttagtgatgtgaaatgctcctgtccctggccctacctccttccctgtccccacccctgcataaggcagttgttggttttcttccccaattcttttccaagtaggttttgtttaccctactccccaaatccctgagccagaagtggggtgcttatactcccaaaccttgagtgtccagccttcccctgttgtttttagtctcttgtgctgtgcctagtggcacctgggctggggaggacactgccccgtctaggtttttataaatgtcttactcaagttcaaacctccagcctgtgaatcaactgtgtctcttttttgacttggtaagcaagtattaggctttggggtggggggaggtctgtaatgtgaaacaacttcttgtctttttttctcccactgttgtaaataacttttaatggccaaaccccagatttgtactttttttttttttctaactgctaaaaccattctcttccacctggttttactgtaacatttgg

SEQ ID NO: 46 - protein, 195 aa, homo sapiens
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 47 - DNA, 662 bp, homo sapiens
  CDS (49)..(456)
accgccgacgcagacccctctctgcacgccagcccgcccgcacccaccatggccacagttcagcagctggaaggaagatggcgcctggtggacagcaaaggctttgatgaatacatgaaggagctaggagtgggaatagctttgcgaaaaatgggcgcaatggccaagccagattgtatcatcacttgtgatggtaaaaacctcaccataaaaactgagagcactttgaaaacaacacagttttcttgtaccctgggagagaagtttgaagaaaccacagctgatggcagaaaaactcagactgtctgcaactttacagatggtgcattggttcagcatcaggagtgggatgggaaggaaagcacaataacaagaaaattgaaagatgggaaattagtggtggagtgtgtcatgaacaatgtcacctgtactcggatctatgaaaaagtagaataaaaattccatcatcactttggacaggagttaattaagagaatgaccaagctcagttcaatgagcaaatctccatactgtttctttcttttttttttcattactgtgttcaattatctttatcataaacattttacatgcagctatttcaaagtgtgttggattaattaggatcatccctttggttaataaataaatgtgtttgtgct

SEQ ID NO: 48 - protein, 135 aa, homo sapiens
MATVQQLEGRWRLVDSKGFDEYMKELGVGIALRKMGAMAKPDCIITCDGKNLTIKTESTLKTTQFSCTLGEKFEETTADGRKTQTVCNFTDGALVQHQEWDGKESTITRKLKDGKLVVECVMNNVTCTRIYEKVE

SEQ ID NO: 49 - DNA, 2808 bp, homo sapiens
  CDS (80)..(610)
ggcggcggcggcaggagcccgggaggcggaggcgggaggcggcggcggcgcgcggagacgcagcagcggcagcggcagcatgtcggccggcggagcgtcagtcccgccgcccccgaaccccgccgtgtccttcccgccgccccgggtcaccctgcccgccggccccgacatcctgcggacctactcgggcgccttcgtctgcctggagattctgttcgggggtcttgtctggattttggttgcctcctccaatgttcctctacctctactacaaggatgggtcatgtttgtgtccgtgacagcgtttttcttttcgctcctctttctgggcatgttcctctctggcatggtggctcaaattgatgctaactggaacttcctggattttgcctaccattttacagtatttgtcttctattttggagcctttttattggaagcagcagccacatccctgcatgatttgcattgcaatacaaccataaccgggcagccactcctgagtgataaccagtataacataaacgtagcagcctcaatttttgcctttatgacgacagcttgttatggttgcagtttgggtctggctttacgaagatggcgaccgtaacactccttagaaactggcagtcgtatgttagtttcacttgtctactttatatgtctgatcaatttggataccattttgtccagatgcaaaaacattccaaaagtaatgtgtttagtagagagagactctaagctcaagttctggtttatttcatggatggaatgttaattttattatgatattaaagaaatggccttttattttacatctctcccctttttccctttccccctttattttcctccttttctttctgaaagtttccttttatgtccataaaatacaaatatattgttcataaaaaattagtatcccttttgtttggttgctgagtcacctgaaccttaattttaattggtaattacagcccctaaaaaaaacacatttcaaataggcttcccactaaactctatattttagtgtaaaccaggaattggcacactttttttagaatgggccagatggtaaatatttatgcttcacggtccatacagtctctgtcacaactattcagttctgctagtatagcgtgaaagcagctatacacaatacagaaatgaatgagtgtggttatgttctaataaaacttatttataaaaacaaggggaggctgggtttagcctgtgggccatagtttgtcaaccactggtgtaaaaccttagttatatatgatctgcattttcttgaactgatcattgaaaacttataaacctaacagaaaagccacataatatttagtgtcattatgcaataatcacattgcctttgtgttaatagtcaaatacttacctttggagaatacttacctttggaggaatgtataaaatttctcaggcagagtcctggatataggaaaaagtaatttatgaagtaaacttcagttgcttaatcaaactaatgatagtctaacaactgagcaagatcctcatctgagagtgcttaaaatgggatccccagagaccattaaccaatactggaactggtatctagctactgatgtcttactttgagtttatttatgcttcagaatacagttgtttgccctgtgcatgaatatacccatatttgtgtgtggatatgtgaagcttttccaaatagagctctcagaagaattaagtttttacttctaattattttgcattactttgagttaaatttgaatagagtattaaatataaagttgtagattcttatgtgtttttgtattagcccagacatctgtaatgtttttgcactggtgacagacaaaatctgttttaaaatcatatccagcacaaaaactatttctggctgaatagcacagaaaagtattttaacctacctgtagagatcctcgtcatggaaaggtgccaaactgttttgaatggaaggacaagtaagagtgaggccacagttcccaccacacgagggcttttgtattgttctactttttcagccctttactttctggctgaagcatccccttggagtgccatgtataagttgggctattagagttcatggaacatagaacaaccatgaatgagtggcatgatccgtgcttaatgatcaagtgttacttatctaataatcctctagaaagaaccctgttagatcttggtttgtgataaaaatataaagacagaagacatgaggaaaaacaaaaggtttgaggaaatcaggcatatgactttatacttaacatcagatcttttctataatatcctactactttggttttcctagctccataccacacacctaaacctgtattatgaattacatattacaaagtcataaatgtgccatatggatatacagtacattctagttggaatcgtttactctgctagaatttaggtgtgagattttttgtttcccaggtatagcaggcttatgtttggtggcattaaattggtttctttaaaatgctttggtggcacttttgtaaacagattgcttctagattgttacaaaccaagcctaagacacatctgtgaatacttagatttgtagcttaatcacattctagacttgtgagttgaatgacaaagcagttgaacaaaaattatggcatttaagaatttaacatgtcttagctgtaaaaatgagaaagtgttggttggttttaaaatctggtaactccatgatgaaaagaaatttattttatacgtgttatgtctctaataaagtattcatttgataaaaaaaaaaaaaaa

SEQ ID NO: 50 - protein, 176 aa, homo sapiens
MSAGGASVPPPPNPAVSFPPPRVTLPAGPDILRTYSGAFVCLEILFGGLVWILVASSNVPLPLLQGWVMFVSVTAFFFSLLFLGMFLSGMVAQIDANWNFLDFAYHFTVFVFYFGAFLLEAAATSLHDLHCNTTITGQPLLSDNQYNINVAASIFAFMTTACYGCSLGLALRRWRP

SEQ ID NO: 51 - DNA, 3097 bp, Homo sapiens
  CDS (187)..(1575)
cccccgagggacatgagagaagaggagcggcgctcaggttattccaggatctttggagacccgaggaaagccgtgttgaccaaaagcaagacaaatgactcacagagaaaaaagatggcagaaccaagggcaactaaagccgtcaggttctgaacagctggtagatgggctggcttactgaaggacatgattcagactgtcccggacccagcagctcatatcaaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttggaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccaggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaagggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcaccc

SEQ ID NO: 52 - protein, 462 aa, Homo sapiens
MIQTVPDPAAHIKEALSVVSEDQSLFECAYGTPHLAKTEMTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTDLPYEPPRRSAWTGHGHPTPQSKAAQPSPSTVPKTEDQRPQLDPYQILGPTSSRLANPGSGQIQLWQFLLELLSDSSNSSCITWEGTNGEFKMTDPDEVARRWGERKSKPNMNYDKLSRALRYYYDKNIMTKVHGKRYAYKFDFHGIAQALQPHPPESSLYKYPSDLPYMGSYHAHPQKMNFVAPHPPALPVTSSSFFAAPNPYWNSPTGGIYPNTRLPTSHMPSHLGTYY